(19) United States
(12) Patent Application Publication
(10) Pub. No.: US 2024/0417802 A1
(43) Pub. Date: Dec. 19, 2024

(54) IMMUNOTHERAPEUTIC METHODS FOR TREATING CANCER

(71) Applicant: THE UNIVERSITY OF CHICAGO, Chicago, IL (US)

(72) Inventors: Jeffrey HUBBELL, Chicago, IL (US); Priscilla BRIQUEZ, Chicago, IL (US); Zoe GOLDBERGER, Chicago, IL (US); Sylvie HAUERT, Chicago, IL (US)

(73) Assignee: THE UNIVERSITY OF CHICAGO, Chicago, IL (US)

(21) Appl. No.: 18/700,817

(22) PCT Filed: Oct. 14, 2022
(86) PCT No.: PCT/US2022/078091

§ 371 Date: Apr. 12, 2024

Related U.S. Application Data
(60) Provisional application No. 63/255,841, filed on Oct. 14, 2021.

(51) Int. Cl. C12Q 1/6886; G01N 33/574

(52) U.S. Cl. CPC C12Q 1/6886; G01N 33/57492; C12Q 2600/156; G01N 2800/52

(57) ABSTRACT

The current disclosure provides for novel therapeutic methods by identifying patient populations that may be treated effectively by immunotherapies. Accordingly, aspects of the disclosure relate to a method for treating cancer in a subject comprising administering to the subject an immunotherapy after a biological sample from the subject has been analyzed for membrane-localized antigens (mAg).

### CROSS-REFERENCE TO RELATED APPLICATIONS

This application claims the benefit of priority of U.S. Provisional Patent Application No. 63/255,841 filed Oct. 14, 2021, which is hereby incorporated by reference in its entirety.

### BACKGROUND

### I. Field of the Invention

This invention relates to the field of biotechnology and therapeutic treatment methods.

### II. Background

Immunotherapies have revolutionised the landscape of clinical oncology, being established as first-line treatments in multiple advanced cancer types, including melanoma, non-small cell lung cancer (NSCLC) and renal cell carcinoma (1-3). Despite the strong efficacy of immune checkpoint immunotherapy (ICI), less than 20% of patients show complete or durable response (4,5). While studies have shown that infiltration of immune cells in the tumours (6) and high tumour mutational burden are key correlates of response to ICI (7-12), accurate prediction of patient responsiveness to ICI remains an important challenge (13). Greater predictivity certainly would increase patient survival and quality of life, by reducing the number, duration and side-effects of treatments as well as associated economic burden. Stratifying patients into those that are likely and unlikely to respond to ICI, based on one or more biomarkers, will provide for more effective and therapeutic treatment methods for patients, since patients can be provided with the most effective therapy before further spreading of the disease.

### SUMMARY OF THE INVENTION

The current disclosure provides for novel therapeutic methods by identifying patient populations that may be treated effectively by immunotherapies. Accordingly, the disclosure relates to a method for treating cancer in a subject comprising administering to the subject an immunotherapy after a biological sample from the subject has been analyzed for membrane-localized antigens (mAg). Also described is a method for treating cancer in a subject comprising administering to the subject an immunotherapy after the mutation status of a gene selected from the genes of Table 1 has been determined in a biological sample from the subject. Also described is a method for prognosing a subject having cancer or for predicting a cancer subject's response to immunotherapy, the method comprising evaluating a biological sample from the subject for mAg. The disclosure also provides for a method for predicting a cancer subject's response to immunotherapy, the method comprising analyzing the mutation status of a gene selected from the genes of Table 1 in a biological sample from the subject. The disclosure also describes a method comprising evaluating mAg in a biological sample from a subject having cancer. Also described as a method comprising evaluating the mutation status of a gene selected from the genes of Table 1 in a biological sample from a subject having cancer. Also described is a method for monitoring a response to an immunotherapy in a subject having cancer comprising: analyzing mAg in a biological sample from the subject before and/or after the subject has been treated with the ICI therapy.

Table 1 refers to the Table below:

TABLE 1
List of Biomarkers

ALK
APC
ATR
BAP1
BRAF
CARD11
CSF3R
EPHA3
EPHA5
EPHA7
EPHB1
ERBB3
ERBB4
ERG
ESR1
FAT1
FGFR1
FGFR4
FLT3
GNA11
GNAQ
IGF1R
IL7R
INPP4A
INSR
KDR
KRAS
MAP2K2
MET
NOTCH1
NOTCH2
NOTCH3
NOTCH4
NTRK2
NTRK3
PDGFRB
PTCH1
PTPRD
PTPRT
RET
RNF43
ROS1
STK11
TGFBR2
TSC2
VHL

The immunotherapy may comprise or consist of immune checkpoint immunotherapy (ICI). Other immunotherapies include activation of co-stimulatory molecules, dendritic cell therapy, CAR-T cell therapy, cytokine therapy, and adoptive T-cell therapy. The immunotherapy may comprise inhibition of co-inhibitory molecules. The methods of the disclosure may exclude one or more of activation of co-stimulatory molecules, dendritic cell therapy, CAR-T cell therapy, cytokine therapy, and adoptive T-cell therapy. The ICI therapy may comprise a monotherapy or a combination ICI therapy. Monotherapy refers to a therapy in which only one ICI therapy is applied, and combination therapy refers to a therapy in which at least two different ICI therapies are applied. The ICI therapy may comprise an inhibitor of PD-1, PDL1, PDL2, CTLA-4, B7-1, B7-2, and combinations thereof. The ICI therapy may comprise an anti-PD-1 monoclonal antibody, an anti-CTLA-4 monoclonal antibody, and combinations thereof. The ICI therapy may comprise one or more of nivolumab, pembrolizumab, pidilizumab, ipilimumab or tremelimumab.

The biological sample may be one that has been determined to have high mAg. The biological sample may be one that has been determined to have high mAg relative to a control, wherein the control is a cut-off value or wherein the control is a level of mAg in a biological sample from a subject or the average level of mAg in biological samples from subjects determined to not have an effective response to immunotherapy.

The cancer may be breast cancer, bladder cancer, cervical cancer, colorectal cancer, head and neck cancer, Hodgkin lymphoma, liver cancer, lung cancer, renal cancer, renal cell cancer, skin cancer, stomach cancer, esophagogastric cancer, glioma, non-small cell lung cancer, melanoma, or rectal cancer. The cancer may be thyroid cancer. The cancer may exclude thyroid cancer, breast cancer, bladder cancer, cervical cancer, colorectal cancer, head and neck cancer, Hodgkin lymphoma, liver cancer, lung cancer, renal cancer, renal cell cancer, skin cancer, stomach cancer, esophagogastric cancer, glioma, non-small cell lung cancer, melanoma, or rectal cancer. The cancer may also be a cancer described herein. The cancer may also comprise a solid tumor.

Methods of the disclosure may comprise or further comprise the administration of at least one additional anticancer treatment. The at least one additional anticancer treatment may comprise or consist of surgical therapy, chemotherapy, radiation therapy, hormonal therapy, immunotherapy, small molecule therapy, receptor kinase inhibitor therapy, anti-angiogenic therapy, cytokine therapy, cryotherapy, or a biological therapy. The anticancer treatment may also be one that is described herein. The subject may be one that is not being treated with a particular anticancer treatment, such as surgical therapy, chemotherapy, radiation therapy, hormonal therapy, immunotherapy, small molecule therapy, receptor kinase inhibitor therapy, anti-angiogenic therapy, cytokine therapy, cryotherapy, a biological therapy, or an anticancer treatment described herein.

Methods of the disclosure may include or further include analyzing, determining, measuring, or evaluating mAg. The biological sample may be one that has been determined to have, measured as having, or evaluated as having high mAg. The biological sample may be one that has been determined to have, measured as having, or evaluated as having high mAg relative to a control. The biological sample may be one that has been determined to have, measured as having, or evaluated as having low mAg. The biological sample may be one that has been determined to have, measured as having, or evaluated as having low mAg relative to a control. The biological sample may be one that has been determined to have, measured as having, or evaluated as having a level of mAg that is not significantly different than a control. Analyzing, evaluating, determining, or measuring mAg may comprise sequencing of nucleic acids in a biological sample from the subject. Analyzing, evaluating, determining, or measuring mAg may comprise performing immunohistochemistry on the biological sample from the subject. The subject may be predicted to respond effectively to the immunotherapy when the biological sample 10 from the subject has been evaluated as having high mAg. The subject may be predicted to respond effectively to the immunotherapy when the biological sample from the subject has been evaluated as having high mAg compared to a control. The subject may be predicted to have a favorable prognosis when the biological sample from the subject has been evaluated as having high mAg. The subject may be predicted to have a favorable prognosis when the biological sample from the subject has been evaluated as having high mAg compared to a control. The subject may be predicted to not respond effectively to the immunotherapy when the biological sample from the subject has been evaluated as having low mAg or a level of mAg that is not significantly different than a control. The subject may be predicted to not respond effectively to the immunotherapy when the biological sample from the subject has been evaluated as having low mAg compared to a control or a level of mAg that is not significantly different than a control. The subject may be predicted to have an unfavorable prognosis when the biological sample from the subject has been evaluated as having low mAg or a level of mAg that is not significantly different than a control. The subject may be predicted to have an unfavorable prognosis when the biological sample from the subject has been evaluated as having low mAg compared to a control or a level of mAg that is not significantly different than a control.

Methods of the disclosure may include or further include analyzing, determining, measuring, or evaluating tumor mutational burden (TMB). The subject may be one that has had TMB analyzed, determined, measured, or evaluated in a biological sample from the subject. The biological sample may be one that has been determined to have high TMB. The biological sample may be one has been determined to have high TMB relative to a control. The biological sample may be one that has been determined to have low TMB. The biological sample may be one that has been determined to have low TMB relative to a control. The biological sample may be one that has been determined to have a level of TMB that is not significantly different than a control. The control may comprise the level of TMB in a biological sample from a subject or the average level of TMB in biological samples from subjects determined to not have an effective response to immunotherapy. The TMB may be determined by sequencing of nucleic acids in or performing immunohistochemistry on a biological sample from the subject. The subject may be predicted to respond effectively to the immunotherapy when the biological sample from the subject has been evaluated as having high TMB. The subject may be predicted to respond effectively to the immunotherapy when the biological sample from the subject has been evaluated as having high mAg and high TMB. The subject may be predicted to respond effectively to the immunotherapy when the biological sample from the subject has been evaluated as having high TMB compared to a control. The subject may be predicted to respond effectively to the immunotherapy when the biological sample from the subject has been evaluated as having high mAg and high TMB compared to a control. The subject may be predicted to have a favorable prognosis when the biological sample from the subject has been evaluated as having high TMB. The subject may be predicted to have a favorable prognosis when the biological sample from the subject has been evaluated as having high mAg and high TMB. The subject may be predicted to have a favorable prognosis when the biological sample from the subject has been evaluated as having high TMB relative to a control. The subject may be predicted to have a favorable prognosis when the biological sample from the subject has been evaluated as having high mAg and high TMB relative to a control. The biological sample may be one that has been evaluated as having low TMB. The subject may be predicted to not respond effectively to the immunotherapy when the biological sample from the subject has been evaluated as having low mAg and low TMB. The subject may be predicted to not respond effectively to the immunotherapy when the biological sample from the subject has been evaluated as having low mAg and low TMB compared to a control. The subject may be predicted to have an unfavorable prognosis when the biological sample from the subject has been evaluated as having low mAg and low TMB. The subject may be predicted to have an unfavorable prognosis when the biological sample from the subject has been evaluated as having low mAg and low TMB compared to a control. The control may comprise the TMB in non-cancerous tissue. The control may comprise the level of TMB in a biological sample from a subject or the average level of TMB in biological samples from subjects determined to not have an effective response to the immunotherapy.

The biological sample may comprise a tissue sample, a cancerous sample, a tumor sample, or a sample obtained from a biopsy. The biological sample may also comprise a biological sample described herein. The biological sample may comprise a blood sample.

The biological sample may be one that has been determined to have or evaluated as having (i) mutant ALK, KDR, PTPRD, FAT1, and/or ERBB3; (ii) non-mutant NOTCH1; or iii) combinations of (i) and/or (ii). The subject may be one that has bladder cancer. The biological sample may be one has been determined to have or evaluated as having mutant ESR1. The subject may have breast cancer. The biological sample may be one has been determined to have or evaluated as having (i) mutant RNF43, NOTCH3, INPP4A, PTPRD, PDGFRB, PTCH1, NOTCH4, FAT1, and/or BRAF; (ii) non-mutant CARD11, APC, and/or CSF3R; or iii) combinations of (i) and/or (ii). The subject may have colorectal cancer. The biological sample may be one that has been determined to have or evaluated as having (i) mutant ATR and/or KRAS; (ii) non-mutant NOTCH1; or iii) combinations of (i) and/or (ii). The subject may have esophagogastric cancer. The biological sample may be one that has been determined to have or evaluated as having (i) mutant FGFR1; (ii) non-mutant TSC2; or iii) combination of (i) and (ii). The subject may have glioma. The biological sample may be one has been determined to have or evaluated as having (i) mutant EPHA7, MAP2K2, and/or EPHB1; (ii) non-mutant ROS1; or iii) combinations of (i) and/or (ii). The subject may have head and neck cancer. The biological sample may be one has been determined to have or evaluated as having (i) mutant FGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2; or iii) combinations of (i) and/or (ii). The subject may be one that has non-small cell lung cancer. The biological sample may be one that has been determined to have or evaluated as having (i) mutant IGF1R, ATR, INSR, NTRK2, CARDi11, ERG, NTRK3, PTPRD, ROS1, and/or PTPRT; (ii) non-mutant BAP1, GNAQ, MAP2K2, and/or GNA11; or iii) combinations of (i) and/or (ii). The subject may have melanoma. The biological sample may be one that has been determined to have or evaluated as having mutant VHL The subject may have renal cell carcinoma.

The biological sample may be one that has been determined to not have or evaluated as not having (i) mutant ALK, KDR, PTPRD, FATi, and/or ERBB3; (ii) non-mutant NOTCH1; or iii) combinations of (i) and/or (ii). The biological sample may be one that has been determined to not have or evaluated as not having mutant ESR1. The biological sample may be one that has been determined to not have or evaluated as not having (i) mutant RNF43, NOTCH3, INPP4A, PTPRD, PDGFRB, PTCH1, NOTCH4, FAT1, and/or BRAF; (ii) non-mutant CARD11, APC, and/or CSF3R; or iii) combinations of (i) and/or (ii). The biological sample may be one that has been determined to not have or evaluated as not having (i) mutant ATR and/or KRAS; (ii) non-mutant NOTCH1; or iii) combinations of (i) and/or (ii). The biological sample may be one that has been determined to not have or evaluated as not having (i) mutant FGFR1; (ii) non-mutant TSC2; or iii) combination of (i) and (ii). The biological sample may be one that has been determined to not have or evaluated as not having (i) mutant EPHA7, MAP2K2, and/or EPHB1; (ii) non-mutant ROS1; or iii) combinations of (i) and/or (ii). The biological sample may be one that has been determined to not have or evaluated as not having (i) mutant FGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2; or iii) combinations of (i) and/or (ii). The biological sample may be one that has been determined to not have or evaluated as not having (i) mutant IGF1R, ATR, INSR, NTRK2, CARD11, ERG, NTRK3, PTPRD, ROS1, and/or PTPRT; (ii) non-mutant BAP1, GNAQ, MAP2K2, and/or GNA11; or iii) combinations of (i) and/or (ii). The biological sample may be one that has been determined to not have or evaluated as not having mutant VHL.

The subject may be predicted to respond effectively to the immunotherapy when the biological sample from the subject has been evaluated as having (i) mutant ALK, KDR, PTPRD, FAT1, and/or ERBB3; (ii) non-mutant NOTCH1; or iii) combinations of (i) and/or (ii). The subject may be predicted to respond effectively to the immunotherapy when the biological sample from the subject has been evaluated as having high TMB and (i) mutant ALK, KDR, PTPRD, FAT1, and/or ERBB3; (ii) non-mutant NOTCH1; or iii) combinations of (i) and/or (ii). The subject may be predicted to not respond effectively to the immunotherapy when the biological sample from the subject has been evaluated as not having (i) mutant ALK, KDR, PTPRD, FAT1, and/or ERBB3; (ii) non-mutant NOTCH1; or iii) combinations of (i) and/or (ii). The subject may be predicted to not respond effectively to the immunotherapy when the biological sample from the subject has been evaluated as having low TMB or a level of TMB that is not significantly different from a control and as not having (i) mutant ALK, KDR, PTPRD, FAT1, and/or ERBB3; (ii) non-mutant NOTCH1; or iii) combinations of (i) and/or (ii). The subject may be predicted to respond effectively to the immunotherapy when the biological sample from the subject has been evaluated as having mutant ESR1. The subject may be predicted to respond effectively to the immunotherapy when the biological sample from the subject has been evaluated as having high TMB and mutant ESR1. The subject may be predicted to not respond effectively to the immunotherapy when the biological sample from the subject has been evaluated as not having mutant ESR1. The subject may be predicted to not respond effectively to the immunotherapy when the biological sample from the subject has been evaluated as having low TMB (or a level of TMB that is not significantly different from a control) and as not having mutant ESR1. The subject may be predicted to respond effectively to the immunotherapy when the biological sample from the subject has been evaluated as having (i) mutant RNF43, NOTCH3, INPP4A, PTPRD, PDGFRB, PTCH1, NOTCH4, FAT1, and/or BRAF; (ii) non-mutant CARD11, APC, and/or CSF3R; or iii) combinations of (i) and/or (ii). The subject may be predicted to respond effectively to the immunotherapy when the biological sample from the subject has been evaluated as having high TMB and as having (i) mutant RNF43, NOTCH3, INPP4A, PTPRD, PDGFRB, PTCH1, NOTCH4, FAT1, and/or BRAF; (ii) non-mutant CARD11, APC, and/or CSF3R; or iii) combinations of (i) and/or (ii). The subject may be predicted to not respond effectively to the immunotherapy when the biological sample from the subject has been evaluated as not having (i) mutantRNF43, NOTCH3, INPP4A, PTPRD, PDGFRB, PTCH1, NOTCH4, FAT1, and/or BRAF; (ii) non-mutant CARD11, APC, and/or CSF3R; or iii) combinations of (i) and/or (ii). The subject may be predicted to not respond effectively to the immunotherapy when the biological sample from the subject has been evaluated as having low TMB (or a level of TMB that is not significantly different from a control) and as not having (i) mutant RNF43, NOTCH3, INPP4A, PTPRD, PDGFRB, PTCH1, NOTCH4, FAT1, and/or BRAF; (ii) non-mutant CARD11, APC, and/or CSF3R; or iii) combinations of (i) and/or (ii). The subject may be predicted to respond effectively to the immunotherapy when the biological sample from the subject has been evaluated as having (i) mutant ATR and/or KRAS; (ii) non-mutant NOTCH1; or iii) combinations of (i) and/or (ii). The subject may be predicted to respond effectively to the immunotherapy when the biological sample from the subject has been evaluated as having high TMB and as having (i) mutant ATR and/or KRAS; (ii) non-mutant NOTCH1; or iii) combinations of (i) and/or (ii). The subject may be predicted to not respond effectively to the immunotherapy when the biological sample from the subject has been evaluated as not having (i) mutant ATR and/or KRAS; (ii) non-mutant NOTCH1; or iii) combinations of (i) and/or (ii). The subject may be predicted to not respond effectively to the immunotherapy when the biological sample from the subject has been evaluated as having low TMB (or a level of TMB that is not significantly different from a control) and as not having (i) mutant ATR and/or KRAS; (ii) non-mutant NOTCH1; or iii) combinations of (i) and/or (ii). The subject may be predicted to respond effectively to the immunotherapy when the biological sample from the subject has been evaluated as having (i) mutant FGFR1; (ii) non-mutant TSC2; or iii) combination of (i) and (ii). The subject may be predicted to respond effectively to the immunotherapy when the biological sample from the subject has been evaluated as having high TMB and as having (i) mutant FGFR1; (ii) non-mutant TSC2; or iii) combination of (i) and (ii). The subject may be predicted to not respond effectively to the immunotherapy when the biological sample from the subject has been evaluated as not having (i) mutant FGFR1; (ii) non-mutant TSC2; or iii) combination of (i) and (ii). The subject may be predicted to not respond effectively to the immunotherapy when the biological sample from the subject has been evaluated as having low TMB (or a level of TMB that is not significantly different from a control) and as not having (i) mutant FGFR1; (ii) non-mutant TSC2; or iii) combination of (i) and (ii). The subject may be predicted to respond effectively to the immunotherapy when the biological sample from the subject has been evaluated as having (i) mutant EPHA7, MAP2K2, and/or EPHB1; (ii) non-mutant ROS1; or iii) combinations of (i) and/or (ii). The subject may be predicted to respond effectively to the immunotherapy when the biological sample from the subject has been evaluated as having high TMB and as having (i) mutant EPHA7, MAP2K2, and/or EPHB1; (ii) non-mutant ROS1; or iii) combinations of (i) and/or (ii). The subject may be predicted to not respond effectively to the immunotherapy when the biological sample from the subject has been evaluated as not having (i) mutant EPHA7, MAP2K2, and/or EPHB1; (ii) non-mutant ROS1; or iii) combinations of (i) and/or (ii). The subject may be predicted to not respond effectively to the immunotherapy when the biological sample from the subject has been evaluated as having low TMB (or a level of TMB that is not significantly different from a control) and as not having (i) mutant EPHA7, MAP2K2, and/or EPHB1; (ii) non-mutant ROS1; or iii) combinations of (i) and/or (ii). The subject may be predicted to respond effectively to the immunotherapy when the biological sample from the subject has been evaluated as having (i) mutant FGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2; or iii) combinations of (i) and/or (ii). The subject may be predicted to respond effectively to the immunotherapy when the biological sample from the subject has been evaluated as having high TMB and as having (i) mutant FGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2; or iii) combinations of (i) and/or (ii). The subject may be predicted to not respond effectively to the immunotherapy when the biological sample from the subject has been evaluated as not having (i) mutant FGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2; or iii) combinations of (i) and/or (ii). The subject may be predicted to not respond effectively to the immunotherapy when the biological sample from the subject has been evaluated as having low TMB (or a level of TMB that is not significantly different from a control) and as not having (i) mutant FGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2; or iii) combinations of (i) and/or (ii). The subject may be predicted to respond effectively to the immunotherapy when the biological sample from the subject has been evaluated as having (i) mutant IGF1R, ATR, INSR, NTRK2, CARD11, ERG, NTRK3, PTPRD, ROS1, and/or PTPRT; (ii) non-mutant BAP1, GNAQ, MAP2K2, and/or GNA11; or iii) combinations of (i) and/or (ii). The subject may be predicted to respond effectively to the immunotherapy when the biological sample from the subject has been evaluated as having high TMB and as having (i) mutant IGF1R, ATR, INSR, NTRK2, CARD11, ERG, NTRK3, PTPRD, ROS1, and/or PTPRT; (ii) non-mutant BAP1, GNAQ, MAP2K2, and/or GNA11; or iii) combinations of (i) and/or (ii). The subject may be predicted to not respond effectively to the immunotherapy when the biological sample from the subject has been evaluated as not having (i) mutant IGF1R, ATR, INSR, NTRK2, CARD11, ERG, NTRK3, PTPRD, ROS1, and/or PTPRT; (ii) non-mutant BAP1, GNAQ, MAP2K2, and/or GNA11; or iii) combinations of (i) and/or (ii). The subject may be predicted to not respond effectively to the immunotherapy when the biological sample from the subject has been evaluated as having low TMB (or a level of TMB that is not significantly different from a control) and as not having (i) mutant IGF1R, ATR, INSR, NTRK2, CARD11, ERG, NTRK3, PTPRD, ROS1, and/or PTPRT; (ii) non-mutant BAP1, GNAQ, MAP2K2, and/or GNA11; or iii) combinations of (i) and/or (ii). The subject may be predicted to respond effectively to the immunotherapy when the biological sample from the subject has been evaluated as having mutant VHL. The subject may be predicted to respond effectively to the immunotherapy when the biological sample from the subject has been evaluated as having high TMB and as having mutant VHL. The subject may be predicted to not respond effectively to the immunotherapy when the biological sample from the subject has been evaluated as not having mutant VHL. The subject may be predicted to not respond effectively to the immunotherapy when the biological sample from the subject has been evaluated as having low TMB (or a level of TMB that is not significantly different from a control) and as not having mutant VHL.

The methods may include analyzing nucleic acids from the biological sample for certain biomarkers. The methods may include analyzing genomic sequences and/or analyzing RNA sequences. The analysis may include comparison to a control, such as genomic DNA or RNA from a non-cancerous sample. The control may be genomic DNA from non-cancerous tissues of the subject. Determining or evaluating the mutation status may comprise or further comprise sequencing nucleic acids isolated from a biological sample from the subject.

Methods of the disclosure may comprise or further comprise administering an immunotherapy to the subject predicted to respond to the immunotherapy. Methods of the disclosure may exclude administering an immunotherapy to the subject predicted to not respond to the immunotherapy.

The subject may be one that is not being treated for cancer. The subject may be one that is being treated for a cancer. The subject may be undergoing treatment for an immunotherapy. The subject may be one that is not being treated with an immunotherapy. The subject may be a mammal. The subject may be a laboratory test animal, such as a mouse, rat, rabbit, dog, cat, horse, or pig. The subject may also be further defined as a human subject.

“Treatment” or treating may refer to any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. The treatment may exclude prevention of the disease.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting 20 essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-I. Membrane-bound antigens enhance melanoma tumour immunogenicity and responsiveness to ICI in mice. B16-F10 melanoma cells (1.5 Million (M)) modified to express membrane-bound or soluble full-length ovalbumin (B16mOVA and B16-OVA, respectively), at high (^{HI}) or low (^{LO}) levels, were injected intradermally in C57BL6 mice. The parental B16-F10 wild-type (WT) cells were used as a control. Where indicated, treatment with 200 μg of anti-PD-1 injected intraperitoneally was given to mice when their tumour volume reached 20-50 mm³ (grey thresholds). a, Tumour growth of the different OVA-expressing B16 cell lines upon injection in vivo (Kruskal-Wallis with Dunn's post-test at day 12). b, Immune cell populations infiltrated in the different tumours at day 10 post-injection analysed by flow cytometry (ANOVA with Tukey's post-test and Brown-Forsythe correction when needed). c, Ex vivo restimulation of OVA-specific CD8⁺ and CD4⁺ T cells in spleen of mice bearing the different OVA-expressing tumours at day 10 post-injection (Kruskal-Wallis with Dunn's post-test). d, Anti-OVA antibody quantification per IgG subtype in the plasma of tumour-bearing mice at day 10 post-injection (AUC: area under the curve; Kruskal-Wallis with Dunn's post-test). e, Tumour growth and associated survival of OVA-expressing tumour-bearing mice treated with anti-PD-1 (log-rank tests with Holm-Bonferroni p-values adjustment). f, B16mOVA^{HI} tumour growth upon depletion of CD8⁺ or/and CD4⁺ T cells with anti-PD1 treatment (Kruskal-Wallis with Dunn's post-test at day 14). g, B16mOVA^{HI} tumour growth upon depletion of NK1.1⁺ or/and CD8⁺ T cells with treatment with anti-PD1 (Kruskal-Wallis with Dunn's post-test at day 20). h, B16mOVA^{HI} tumour growth in MuMt⁻ mice (lacking mature B cells) with treatment with anti-PD-1 (Kruskal-Wallis with Dunn's post-test at day 17). i, Tumour growth of mice that survived B16mOVA^{HI} tumours treated with anti-PD-1 upon rechallenge with 250k B16-F10 WT cells (Mann-Whitney test at day 14).

FIG. 2A-F. Neoantigen localisation at the membrane correlates with an increased survival in cancer patients treated with ICI in a pan-cancer analysis. Data available from Samstein et al. (7). Patients suffering from 9 different cancer types were treated with immune checkpoint inhibitor (ICI) immunotherapy, and their survival was evaluated from the first day of treatment (N=1609 patients). A control cohort of patients non-treated with immunotherapy was used for comparison (N=3142 patients). All Kaplan-Meier survival curves and Cox hazard ratios (HR) for survival were statistically compared using log-rank tests. a, Graphical representation of the workflow for the analysis of subcellular localisations associated with the tumour mutations. b, Survival of patients with high (Top 25% group) or low (Bottom 50%, 25% or 10% groups) proportion of membrane-localised neoantigens. c, Survival curves of patients having high (Top 25% group) or low (Bottom 25% group) proportions of neoantigens located in the cytoplasm, in the nucleus or secreted. d, Survival of patients as a function of their predominant subcellular location of neoantigens (Top 25% groups of membrane, nucleus or cytoplasm mutations; p-values adjusted using Holm-Bonferroni correction). e, Survival of non-ICI-treated patients that have high (Top 25%) or low (Bottom 25%) proportion of membrane neoantigens (cutoff values represents a proportion of membrane neoantigens). f, HR for survival of patients having high (Top 25%) versus low (Bottom 25%) proportion of membrane neoantigens upon ICI treatment, non-treated with immunotherapy (Non-ICI), or depending on the type of ICI received, i.e. PD-1/PDL-1, CTLA-4 or in combination (HR±95% CI).

FIG. 3A-B. Membrane neoantigen proportion correlates with increased survival in multiple cancer types. a, Distribution of membrane neoantigen proportion by cancer types (grey line: cutoff value (CV) for the pan-cancer upper quartile: cutoff value for the pan-cancer lower quartile; Kruskall-Wallis test with Dunn's post-tests for comparisons to the pan-cancer group). b, Heatmap of the HR for survival of patients harbouring high versus low neoantigen load per subcellular location and per cancer types. High and low groups are determined using either the cutoff values (CV) from the pan-cancer group or the upper and lower quartiles (25%) specific to the cancer type (log-rank tests).

FIG. 4A-D. Membrane neoantigen proportion correlates with better responsiveness to cancer ICI. Data available from Hellman et al. (8). Patients (N=75) with non-small cell lung cancer were treated with a combination of anti-PD-1 and anti-CTLA-4 immunotherapy, and their responsiveness to treatment was evaluated (responders: complete or partial response (CR/PR); non-responders: stable disease or progressive disease (SD/PD)). a, Proportion of membrane neoantigens in patients that responded or not to the immunotherapy (Mann-Whitney test). b, Proportion of responders and non-responders in patients with high (Top 25%) or low (Bottom 25%) membrane neoantigen load (Fisher's exact test). c, Proportion of neoantigens at the cell plasma membrane or in other specific membrane-containing cell organelles in responders and non-responders to immunotherapy. d, Heatmap of the HR for survival comparing the Top vs. Bottom 50%, 25% or 10% groups having neoantigens at the plasma membrane or in other membrane-containing organelles, from the cohorts from Samstein et al. (7) (pan-cancer group), Hellman et al. (8) and Hugo et al. (9).

FIG. 5A-H. Membrane-localised neoantigens as potent biomarkers for ICI in the clinic. The ICI- and non-ICI-treated cohorts from Samstein et al. (7) were analysed to determine which mAg or combination of mAg were the most potent to predict survival upon ICI. a, HR of survival associated with specific membrane protein-encoding genes per cancer type. A HR<1 indicates that the mutated version of the gene correlates with increased patient survival as compared to the wild-type gene. Corresponding gene-specific HRs from the non-ICI treated cohort are in grey (log-rank test, *p<0.05). Patient coverage indicates the proportion of patient that contains at least one of the mutated genes. b, Survival curves of ICI and non-ICI treated patients bearing NOTCH3 mutations in colorectal cancer. c, Survival curves of ICI and non-ICI treated patients bearing NTRK3 mutations in NSCLC. d, List of selected membrane protein-encoding genes that are currently recognised by the FDA as biomarkers predictive of a response to FDA approved drug according to the OncoKB database (24). e, Comparison of survival of patients carrying BRAF mutations in colorectal cancer treated with ICI or with the FDA-approved encorafenib+cetuximab therapy (data from Kopetz et al. (25)). f, Comparison of survival of patients carrying MET mutations in NSCLC treated with ICI or with the FDA-approved tepotinib therapy (data from Paik et al. (26)). g, Comparison of survival of patients with high or low proportion of mAg for different levels of TMB (TMB≥10 mut/Mbp being the FDA-validated cutoff for ICI treatment for solid tumours (28)) in a pan-cancer analysis. h, Survival curves comparing patients with low TMB (<10 mut/Mbp) and high mAg to patients with high TMB (between 10-20 mut/Mbp) and low mAg, in a pan-cancer analysis. No statistically significant difference was observed between the two groups (log-rank test).

FIG. 6A-H. Characterization of OVA-expressing B16-F10 melanoma cell lines and tumours. a, Design of the different OVA-expressing B16-F10 cell lines, expressing membrane OVA (mOVA) or soluble OVA. b, OVA expression in the modified B16 cell lines in culture in vitro, assessed by qPCR (Mean+SD, ANOVA with Sidak's post-test). c, OVA expression in the modified B16 tumours in vivo, assessed by qPCR (Mean+SD, ANOVA with Sidak's post-test). d, Cell-surface staining of OVA quantified by flow cytometry via the mean fluorescence intensity. e, Detection of cell plasma membrane-bound OVA on the different OVA-expressing B16 cell lines assessed by microscopy (red: anti-OVA; scale bar=50 m). f, Western blot analysis for OVA detection in the extracellular vesicles (EV) produced in vitro by B16mOVA^{HI} or B16-OVA^{HI} cells lines or in the non-EV fraction (black=positive detection of OVA). g, Survival of mice injected with the different OVA-expressing cell lines, associated to the tumour growth curves of FIG. 1a (log-rank tests with Holm-Bonferroni p-values adjustment). h, Tumour growth of B16mOVA^{HI} in Act-mOVA mice as compared to growth in wild-type (WT) mice (mean±SEM; Kruskal-Wallis with Dunn's post-test at day 10).

FIG. 7. Gating strategy for the characterisation of T and NK cells. Multi-colored flow cytometry was used to analyse the subsets of T and NK cells in the tumours at day 10 post-injection. Subset of immune cells were defined using the following markers: NK cells (FSC^{LO}SSC^{LO}, CD45⁺, NK1.1⁺, CD3ε⁻), NK T cells (FSC^{LO}, SSC^{LO}, CD45⁺, CD3ε⁺, NK1.1⁺), CD8⁺ T cells (FSC^{LO}, SSC^{LO}, CD45⁺, NK1.1, CD3ε⁻, CD8⁺), CD4⁺ T cells (FSC^{LO}, SSC^{LO}, CD45⁺, NK1.1⁻, CD3ε⁺, CD8⁺), effector T cells (same markers than T cells with CD44⁺, CD62L⁻), effector memory T cells (same markers than T cells with CD44⁺, CD62L⁺). regulatory T cells (same than, CD4⁺ T cells with CD25⁺, FoxP3⁺).

FIG. 8. Gating strategy for the characterisation of B cells and myeloid cell subsets. Multi-colored flow cytometry was used to analyse the subsets of B cells and myeloid cells in the tumours at day 10 post-injection. Subset of immune cells were defined using the following markers: Macrophages (CD45⁺, F4/80⁺, CD11b⁺), Granylocytic myeloid-derived suppressor cells (MDSC) (CD45⁺, F4/80⁻, CD3ε⁺, Ly6G⁺, Ly6C^{MID/HI}), Monocytic MDSC (CD45⁺, F4/80⁻, CD3ε⁻, Ly6G⁻, Ly6C^{HI}), B cells (CD45⁺, F4/80⁻, CD3ε⁻, Ly6G⁻, Ly6C^{LO/MID}, CD19⁺, B220⁺), dendritic cells (DCs) (CD45⁺, F4/80⁻, CD3ε⁻, Ly6G⁻, Ly6C^{LO/MID}, CD11c⁺, MHCII⁺), CD11b⁺DCs (same than DCs with CD11b⁺), CD103⁺DCs (same than DCs with CD11b⁻, B220⁻, CD103⁺).

FIG. 9A-H. Comparison of B16mOVA and B16-OVA melanoma tumour immunogenicity and responsiveness to cancer immunotherapy in mice. a-e, Flow cytometry analysis of immune cells infiltrated in tumours 10 days post-injection (Mean±SD, ANOVA with Tukey's post-test and Brown-Forsythe correction when needed). a, Number of CD45⁺ immune cells and CD8⁺ T cells per mg of tumour. b, CD8⁺ and c, CD4⁺ effector and effector memory T cells subsets in the different tumours. d, Proportion of PD-1 expressing CD8⁺ and CD4⁺ T cells. e, Proportion of NK T cells, B cells, dendritic cells, macrophages and myeloid-derived suppressor cells relative to the total CD45⁺ immune cell populations. f, Titers (log₁₀) of the anti-OVA measured per IgG subtype in the plasma of tumour-bearing mice at day 10, which corresponds FIG. 1d. (Mean+SD; Kruskal-Wallis with Dunn's post-test per IgG subtype). g, Tumour growth and associated survival of OVA-expressing tumour-bearing mice treated with 100 μg of anti-PD-L1 and 100 μg of anti-CTLA-4 injected intraperitoneally when the tumour volume reached 20-50 mm³ (grey thresholds). h, Survival of mice re-challenged with B16-F10 WT tumour cells, associated to the tumour growth curves presented in FIG. 1i.

FIG. 10A-E. Analysis of tumour neoantigen subcellular localisations and their subsequent impact on patient survival. a, Number of tumour mutated genes associated with each subcellular location among the 469 genes sequenced by MSK-IMPACT method. b, Proportion of tumour mutated genes per subcellular location in patients treated with immunotherapy in the pan-cancer group, and corresponding percentile cutoff values used for the analysis in FIG. 2. c, Survival of ICI-treated patients harbouring high (Top 50% or 10%) or low (Bottom 50% or 10%) proportion of membrane neoantigens (log-rank tests). d, Increase in risk of death as a function of the proportion of membrane neoantigens in ICI-treated patients in the pan-cancer group. Values are calculated as 100*(HR-1)±95% CI with HR the hazard ratio for survival of patients that have less than the depicted proportion as compared to those that have more. As an example, ICI-treated patients that had less than Q1=23% of neoantigens at the membrane had a 50% increased risk of death as compared to those that have more than 23% membrane neoantigens (log-rank tests, values=p-value≤0.05, grey values=not significant). e, Survival of ICI-treated patients with high (Top 25%) proportion of membrane or secreted neoantigens (log-rank test).

FIG. 11A-C. Distributions of patients by cancer types and according to their proportion of membrane neoantigens. a, Distribution of the ICI-treated patients per cancer type included in the pan-cancer analysis. b, Differences in patient distribution per cancer type for the groups with high (Top 25%) or low (Bottom 25%) proportion of membrane neoantigens, as compared to the distribution of the entire ICI-treated cohort as in panel a. c, Values of the HRs, 95% confidence intervals, p-value of the log-rank tests and number of patients used in FIG. 3b, for the universal cutoff and the 25% top vs. bottom strategies.

FIG. 12A-I. Response to immunotherapy based on the proportion of neoantigens at specific subcellular localisations. Patients (N=75) with non-small cell lung cancer were treated with anti-PD-1⁺ anti-CTLA-4 in the cohort from Hellman et al. (8), and patients (N=38) with advanced melanoma cancer were treated with anti-PD-1 in the cohort from Hugo et al. (9). In both studies, tumour mutated genes were sequenced by the WES method. a, Number of tumour mutated genes detected across all patients in the Hellman et al. and Hugo et al. studies, respectively, and their associated subcellular locations. b, Comparison of the proportions of membrane neoantigens found in ICI-treated patient cohorts from the studies by Samstein et al. (7), Hellman et al. (8) and Hugo et al. (9) c, Proportion of neoantigens per subcellular location in patients that responded or not to immunotherapy in the Hellman et al. cohort (Mann-Whitney test). d, Survival of patients with high (Top 25% and 50%) or low (Bottom 25% and 50%) membrane neoantigen proportion in the Hellman et al. (8) cohort (log-rank test). e, Same as in panel c, but with the patient cohort from Hugo et al (9) f, Proportion of responders or non-responders to anti-PD-1 among patients that have high (Top 25%) or low (Bottom 25%) membrane neoantigen proportion in the Hugo et al. (9) cohort (Fisher's exact test). g, Same as in panel d, but with the patient cohort from Hugo et al. (9). h, Proportion of neoantigens at the cell plasma membrane or in other specific membrane-containing cell organelles in responders and non-responders to immunotherapy from the Hugo et al. (9) i, Survival of the patients with high (Top 10%) or low (Bottom 10%) proportion of neoantigens at the tumor cell plasma membrane for the pan-cancer groups from the cohort from Samstein et al. (7)

FIG. 13A-F. Potential use of mAg as predictive clinical biomarkers for extended survival upon ICI. Data analysed from Samstein et al.⁷ ICI-treated or non-ICI-treated cohorts. a, Survival of patients bearing at least one mutated genes among the cancer-specific list of genes in FIG. 5a, as compared to patients with no mutated genes among the list. b, Survival curves of ICI and non-ICI treated patients bearing RNF43 mutations in colorectal cancer. c, Survival curves of ICI and non-ICI treated patients bearing NOTCH1 mutations in NSCLC. d, Comparison of survival of patients carrying RET mutations in NSCLC treated with ICI or with a standard-of-care cabozantinib (data from Gautschi et al. (25)). e, Survival curves of patients from the pan-cancer group in function of their TMB level (in mut/Mbp). The higher the TMB the longer the survival (log-rank test). f, Correlation between TMB and proportion of mAg. No correlation was observed between these two parameters (Spearman correlation).

FIG. 14. Intratumoral cytokine concentration in the B16mOVA^{HI}, B16-OVA^{HI} or wild-type B16 at day 10 after tumor implantation. Quantification was performed by LegendPlex. Values on the graphs indicate statistical p-values (ANOVA tests). P-values<0.05 are considered significant. Differences were observed between B16mOVA^{HI} and B16-OVA^{HI} for IFNγ, CCL4, CCL3, CXCL9, CXCL10 and TNFα. Differences were observed between B16mOVA^{HI} and B16 WT for IFNγ, CCL4, CCL3, VEGF, CXCL10 and TNFα.

FIG. 15. Survival (top) and tumor growth curves of B16mOVA^{HI} tumor-bearing mice to anti-PD-1 checkpoint blockade therapies injected intravenously at day 7, in the WT mouse (left) or the BatF3^{-/-} (right) mouse. Mice that were not injected with PD-1 exhibits shorter survival than mice treated with PD-1, in both WT and BatF3^{-/-} mice. The majority of mice treated with PD-1 were responsive (4/5 complete response) and showed rejection of the tumor, in both WT and BatF3^{-/-} mice.

### DETAILED DESCRIPTION OF THE INVENTION

Due to their genetic instability, tumour cells bear mutations, known as neoantigens, that can effectively be recognised by the immune system. In the clinic, immune checkpoint immunotherapy (ICI) can successfully re-activate immune reactions against tumour neoantigens, leading to remarkable remission in cancer patients. Nevertheless, only a minority of patients are responsive to ICI, and approaches for prediction of responsiveness remain elusive yet are needed to improve the success of cancer treatments. While the number of tumour neoantigens correlates positively with responsiveness and survival of patients undergoing ICI therapy, the influence of the subcellular localisations of these neoantigens in the tumour cell has not been elucidated. Here, the inventors hypothesised that the immune reactions are modulated by the localisation of neoantigens and, therefore, that some neoantigen subcellular localisations could favour responsiveness to ICI. The inventors show in both a mouse melanoma model and human clinical datasets of 1722 ICI-treated patients that high proportions of membrane-localised neoantigens (mAg), particularly at the plasma membrane, correlate with responsiveness to ICI therapy and improved overall survival across multiple cancer types. The inventors further highlight that mutations in the membrane proteins encoded by NOTCH3, RNF43, NTRK3 and NOTCH1, among others, may serve as potent biomarkers to predict extended survival upon ICI in certain cancer types. The inventors anticipate that these results will improve the predictability of cancer patient response to ICI and therefore may have important implications to establish future clinical guidelines to direct the choice of treatment toward ICI.

### I. METHODS FOR EVALUATING MEMBRANE ANTIGENS, TMB, AND GENE MUTATION STATUS

Methods of the disclosure include sequencing of nucleic acid molecules and/or one or more additional assay methods described herein and below. The sequencing method may be performed on RNA or DNA.

### A. Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

### B. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLiD platform, which is now owned by Life Technologies.

### C. 454 Pyrosequencing.

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

### D. Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massivelly parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

### E. Solid Sequencing.

Applied Biosystems' (now a Thermo Fisher Scientific brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

### F. Ion Torrent Semiconductor Sequencing.

Ion Torrent Systems Inc. (now owned by Thermo Fisher Scientific) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

### G. DNA Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects.

### H. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

### I. Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

### J. Additional Assay Methods

In some embodiments, methods involve amplifying and/or sequencing one or more target genomic regions using at least one pair of primers specific to the target genomic regions. In certain embodiments, the primers are heptamers. In other embodiments, enzymes are added such as primases or primase/polymerase combination enzyme to the amplification step to synthesize primers.

In some embodiments, arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface can be used, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

In addition to the use of arrays and microarrays, it is contemplated that a number of difference assays could be employed to analyze nucleic acids. Such assays include, but are not limited to, nucleic amplification, polymerase chain reaction, quantitative PCR, RT-PCR, in situ hybridization, digital PCR, dd PCR (digital droplet PCR), nCounter (nanoString), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), next generation RNA sequencing, northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a genomic region, biomarker, probe, or oligo described herein. The term “primer” or “probe” as used herein, is meant to encompass any nucleic acid that is capable of priming the synthesis of a nascent nucleic acid in a template-dependent process and/or pairing with a single strand of an oligo of the disclosure, or portion thereof. Typically, primers are oligonucleotides from ten to twenty and/or thirty nucleic acids in length, but longer sequences can be employed. Primers may be provided in double-stranded and/or single-stranded form, although the single-stranded form is preferred.

The use of a probe or primer of between 13 and 100 nucleotides, particularly between 17 and 100 nucleotides in length, or up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In one embodiment, quantitative RT-PCR (such as TaqMan, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In one embodiment, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in some samples is that they are of variable quantity and/or quality. This problem can be overcome if the RT-PCR is performed as a relative quantitative RT-PCR with an internal standard in which the internal standard is an amplifiable DNA fragment that is similar or larger than the target DNA fragment and in which the abundance of the DNA representing the internal standard is roughly 5-100 fold higher than the DNA representing the target nucleic acid region.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target DNA fragment. In addition, the nucleic acids isolated from the various samples can be normalized for equal concentrations of amplifiable DNAs.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some embodiments, the density may be or may be at least 50, 100, 200, 300, 400, 500 or more probes/cm2 (or any range derivable therein).

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Certain embodiments may involve the use of arrays or data generated from an array. Data may be readily available. Moreover, an array may be prepared in order to generate data that may then be used in correlation studies.

### II. IMMUNOTHERAPY

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

### A. Immune Checkpoint Immunotherapy

Embodiments of the disclosure may include administration of ICI, which are further described below.

### 1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-i” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. The PD-1 ligand binding partners may be PDL1 and/or PDL2. In another embodiment, a PDL1 inhibitor is a molecule that inhibits the binding of PDL1 to its binding partners. The PDL1 binding partners may be PD-1 and/or B7-1. In another embodiment, the PDL2 inhibitor is a molecule that inhibits the binding of PDL2 to its binding partners. The PDL2 binding partner may be PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDIO680, also known as AMP-514, and REGN2810.

In some embodiments, the ICI therapy comprises a PDL1 inhibitor such as Durvalumab, also known as MEDI4736, atezolizumab, also known as MPDL3280A, avelumab, also known as MSB00010118C, MDX-1105, BMS-936559, or combinations thereof. The ICI therapy may comprise a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### 2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the ICI therapy comprises an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as an ICI therapy in the methods and compositions of the disclosure is ipilimumab (also known as 10D1, MDX-010, MDX-101, and Yervoy®) or antigen binding fragments and variants thereof (see, e.g., WO0 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### B. Activation of Co-Stimulatory Molecules

In some embodiments, the immunotherapy comprises an activator of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### C. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### D. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

### E. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### F. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.[60]

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### III. ADDITIONAL THERAPIES

The current methods and compositions of the disclosure may include one or more additional therapies known in the art and/or described herein. In some embodiments, the additional therapy comprises an additional cancer treatment. Examples of such treatments are described herein, such as the immunotherapies described herein or the additional therapy types described in the following.

### A. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

### B. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### C. Neoantigens

In some embodiments, the additional therapy comprises neoantigen administration. Many tumors express mutations. These mutations potentially create new targetable antigens (neoantigens) for use in T cell immunotherapy. The presence of CD8⁺ T cells in cancer lesions, as identified using RNA sequencing data, is higher in tumors with a high mutational burden. The level of transcripts associated with cytolytic activity of natural killer cells and T cells positively correlates with mutational load in many human tumors.

### D. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dacarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydrazine derivatives (e.g., procarbazine), and adrenocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluorouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2- to 10,000-fold less than the chemotherapeutic effective dose of the chemotherapeutic agent. For example, the chemotherapeutic agent may be administered in an amount that is about 20-fold less, about 500-fold less or even about 5000-fold less than the effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### E. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Grays (Gy) and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### F. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### G. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. Nucleic Acid Assays

Aspects of the methods include assaying nucleic acids to determine expression or activity levels and/or the presence of mAG, TMB, and biomarkers in a biological sample. Arrays can be used to detect differences between two samples. Specifically contemplated applications include identifying and/or quantifying differences between RNA from a sample that is normal and from a sample that is not normal, between a cancerous condition and a non-cancerous condition. Also, RNA may be compared between a sample believed to be susceptible to a particular disease or condition and one believed to be not susceptible or resistant to that disease or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of, or susceptibility to, a disease or condition of which a component is or may or may not be genetic or caused by a hyperproliferative or neoplastic cell or cells.

To determine expression levels of a biomarker, an array may be used. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

Further assays useful for determining biomarker expression include, but are not limited to, nucleic amplification, polymerase chain reaction, quantitative PCR, RT-PCR, in situ hybridization, Northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

A further assay useful for quantifying and/or identifying nucleic acids, such as nucleic acids comprising biomarker genes, is RNAseq. RNA-seq (RNA sequencing), also called whole transcriptome shotgun sequencing, uses next-generation sequencing (NGS) to reveal the presence and quantity of RNA in a biological sample at a given moment in time. RNA-Seq is used to analyze the continually changing cellular transcriptome. Specifically, RNA-Seq facilitates the ability to look at alternative gene spliced transcripts, post-transcriptional modifications, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

### V. PROTEIN ASSAYS

A variety of techniques can be employed to measure expression levels of polypeptides and proteins in a biological sample to determine biomarker expression levels. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining protein expression levels of biomarkers.

In one aspect, antibodies, or antibody fragments or derivatives, can be used in methods such as Western blots, ELISA, flow cytometry, or immunofluorescence techniques to detect levels of mAg, TMB, and biomarkers. In some aspects, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some aspects, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 125I, 3H, and 131I. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73: 147-166 (1981).

### VI. SAMPLE PREPARATION

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple samples may be obtained for diagnosis by the methods described herein. In other cases, multiple samples, such as one or more samples from one tissue type and one or more samples from another specimen (for example blood) may be obtained for diagnosis by the methods. In some cases, multiple samples such as one or more samples from one tissue type and one or more samples from another specimen (e.g. blood) may be obtained at the same or different times. Samples may be obtained at different times are stored and/or analyzed by different methods. For example, a sample may be obtained and analyzed by routine staining methods or any other cytological analysis methods.

In some embodiments, the sample comprises a fractionated sample, such as a blood sample that has been fractionated by centrifugation or other fractionation technique. The sample may be enriched in white blood cells or red blood cells. In some embodiments, the sample may be fractionated or enriched for leukocytes or lymphocytes. In some embodiments, the sample comprises a whole blood sample.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### VII. ADMINISTRATION OF THERAPEUTIC COMPOSITIONS

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VIII. METHODS OF TREATMENT

Provided herein are methods for treating or delaying progression of cancer in an subject through the administration of therapeutic compositions.

In some embodiments, the therapies result in a sustained response in the individual after cessation of the treatment. The methods described herein may find use in treating conditions where enhanced immunogenicity is desired such as increasing tumor immunogenicity for the treatment of cancer.

In some embodiments, the individual has cancer that is resistant (has been demonstrated to be resistant) to one or more anti-cancer therapies or immunotherapies. In some embodiments, resistance to anti-cancer therapy includes recurrence of cancer or refractory cancer. Recurrence may refer to the reappearance of cancer, in the original site or a new site, after treatment. In some embodiments, resistance to anti-cancer therapy includes progression of the cancer during treatment with the anti-cancer therapy. In some embodiments, the cancer is at early stage or at late stage.

In some embodiments of the methods of the present disclosure, the cancer has low levels of T cell infiltration. In some embodiments, the cancer has no detectable T cell infiltrate. In some embodiments, the cancer is a non-immunogenic cancer (e.g., non-immunogenic colorectal cancer and/or ovarian cancer). Without being bound by theory, the combination treatment may increase T cell (e.g., CD4+ T cell, CD8+ T cell, memory T cell) priming, activation, proliferation, and/or infiltration relative to prior to the administration of the combination.

The cancer may be a solid tumor, metastatic cancer, or non-metastatic cancer. In certain embodiments, the cancer may originate in the thyroid, bladder, blood, bone, bone marrow, brain, breast, urinary, cervix, esophagus, duodenum, small intestine, large intestine, colon, rectum, anus, gum, head, kidney, liver, lung, nasopharynx, neck, ovary, prostate, skin, stomach, testis, tongue, or uterus.

The cancer may specifically be of the following histological type, though it is not limited to these: neoplasm, malignant; carcinoma; undifferentiated, bladder, blood, bone, brain, breast, urinary, esophageal, thymomas, duodenum, colon, rectal, anal, gum, head, kidney, soft tissue, liver, lung, nasopharynx, neck, ovary, prostate, skin, stomach, testicular, tongue, uterine, thymic, cutaneous squamous-cell, noncolorectal gastrointestinal, colorectal, melanoma, Merkel-cell, renal-cell, cervical, hepatocellular, urothelial, non-small cell lung, head and neck, endometrial, esophagogastric, small-cell lung mesothelioma, ovarian, esophagogastric, glioblastoma, adrencorical, uveal, pancreatic, germ-cell, giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; cutaneous melanoma, blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer comprises cutaneous squamous-cell carcinoma, noncolorectal and colorectal gastrointestinal cancer, Merkel-cell carcinoma, anal cancer, cervical cancer, hepatocellular cancer, urothelial cancer, melanoma, lung cancer, non-small cell lung cancer, small cell lung cancer, head and neck cancer, kidney cancer, bladder cancer, Hodgkin's lymphoma, pancreatic cancer, or skin cancer.

In some embodiments, the cancer comprises lung cancer, pancreatic cancer, metastatic melanoma, kidney cancer, bladder cancer, head and neck cancer, or Hodgkin's lymphoma.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

In certain aspects, further cancer or metastasis examination or screening, or further diagnosis such as contrast enhanced computed tomography (CT), positron emission tomography-CT (PET-CT), and magnetic resonance imaging (MRI) may be performed for the detection of cancer or cancer metastasis in patients determined to have a certain gut microbiome composition.

### IX. KITS

Certain aspects of the present invention also concern kits containing compositions of the invention or compositions to implement methods of the invention. In some embodiments, kits can be used to evaluate mAg levels and/or mutations of genes. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules, detection agents, antibodies or inhibitors, or any value or range and combination derivable therein. In some embodiments, there are kits for evaluating mAg level, composition, surface markers, genomic sequences, or size in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments. In addition, a kit may include a sample that is a negative or positive control for mAg isolation, characterization, or levels.

It is contemplated that any method or composition described herein can be implemented with respect to any other method or composition described herein and that different embodiments may be combined. The claims originally filed are contemplated to cover claims that are multiply dependent on any filed claim or combination of filed claims.

Embodiments of the disclosure include kits for analysis of a pathological sample by assessing mAg or gene mutation status for a sample comprising, in suitable container means, two or more probes or detection agents, wherein the probes or detection agents detect one or more markers identified herein.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Membrane-Localised Neoantigens Predict the Efficacy of Cancer Immunotherapy

Immunotherapies have revolutionised the landscape of clinical oncology, being established as first-line treatments in multiple advanced cancer types, including melanoma, non-small cell lung cancer (NSCLC) and renal cell carcinoma (1-3). Despite the strong efficacy of immune checkpoint immunotherapy (ICI), less than 20% of patients show complete or durable response (4,5). While studies have shown that infiltration of immune cells in the tumours (6) and high tumour mutational burden are key correlates of response to ICI (7-12), accurate prediction of patient responsiveness to ICI remains an important challenge (13). Greater predictivity certainly would increase patient survival and quality of life, by reducing the number, duration and side-effects of treatments as well as associated economic burden.

Here, the inventors hypothesised that the potency of immune response against tumour neoantigens not only depends on the total mutational burden, but also on the subcellular localisation of the neoantigens within the tumour cell. Indeed, the efficiency of neoantigen presentation on the major histocompatibility complex (MHC)-I by the tumour cell, required for recognition and killing by CD8⁺ T cells (14), might vary for cytoplasmic, nuclear, membrane-localised or secreted antigens due to their specific intracellular processing and trafficking routes (15-17). In addition, efficiency in neoantigen collection and presentation by antigen-presenting cells (APCs), on both MHC-I and -II to generate CD8⁺ and CD4⁺ T cell responses respectively, could similarly be impacted by these different forms of neoantigens upon release in debris or in the extracellular milieu. Apart from antigen presentation, membrane-bound neoantigens can be recognised by antibodies, induced via B cell immunity, which could allow antibody-dependent cytotoxic mechanisms that kill tumour cells by activating natural killer (NK) cells, macrophages or the immune complement cascade (18,19).

To date, very few reports have examined how the subcellular localisation of tumour neoantigen modulates anti-cancer immunity. In this study, the inventors show that membrane-bound tumour neoantigens (mAg) increase tumour immunogenicity and improve responsiveness to ICI therapies. The inventors first demonstrated in a mouse model of melanoma that membrane-localisation of OVA (mOVA; used here as a model neoantigen) in B16-F10 cells increased local and systemic immunity as compared to soluble OVA and rendered these tumours highly susceptible to ICI, in a manner that did not depend on immunoglobulin G (IgG) antibody-mediated cytotoxicity. The inventors then questioned if a high burden of membrane-localised neoantigens improves responsiveness to ICI in cancer patients. The inventors developed a simple algorithm that extracts the subcellular localisations associated with tumour mutated genes from the UniprotKB/Swiss-Prot database (20) and analysed the publicly available sequencing data of 4864 patients, treated or not with ICI, from studies by Samstein et al. (7), Hellman et al. (8) and Hugo et al. (9). The inventors demonstrated that a high proportion of membrane-localised neoantigens correlates with increased patient survival and responsiveness to ICI across multiple cancer types. Moreover, the inventors highlighted that mutated genes encoding for some particular mAg may serve as potent biomarkers to predict extended survival of patients upon ICI, such as NOTCH1, NOTCH3, RNF43 or NTRK3. Together, these results highlight the importance of considering the subcellular localisation of tumour neoantigens, in addition to the overall tumour mutational burden (TMB), to improve the predictivity of patient responsiveness to ICI therapy and potentially the clinical guidelines for the selection of the most appropriate cancer treatment. Such findings may also have strong implications on vaccinal antigen selection for neoantigen-targeted cancer vaccines based on tumour gene sequencing.

### A. Results

### 1. mAg Increase Tumour Immunogenicity

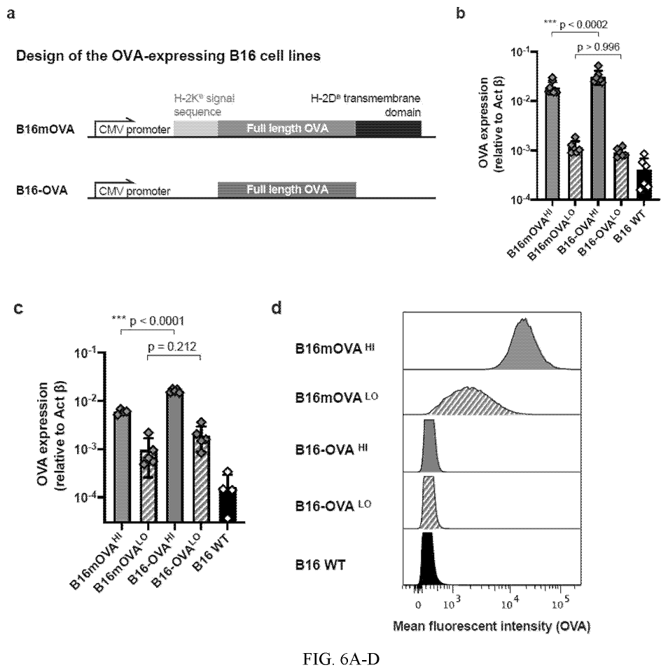

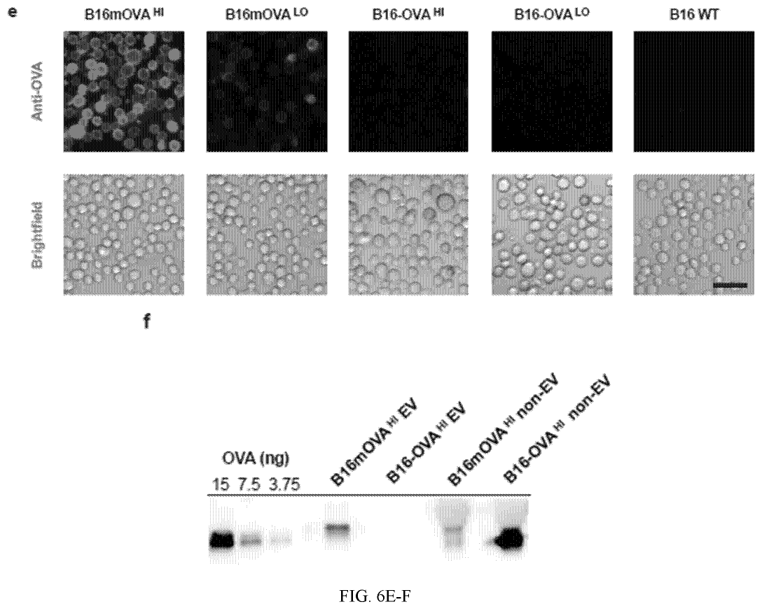

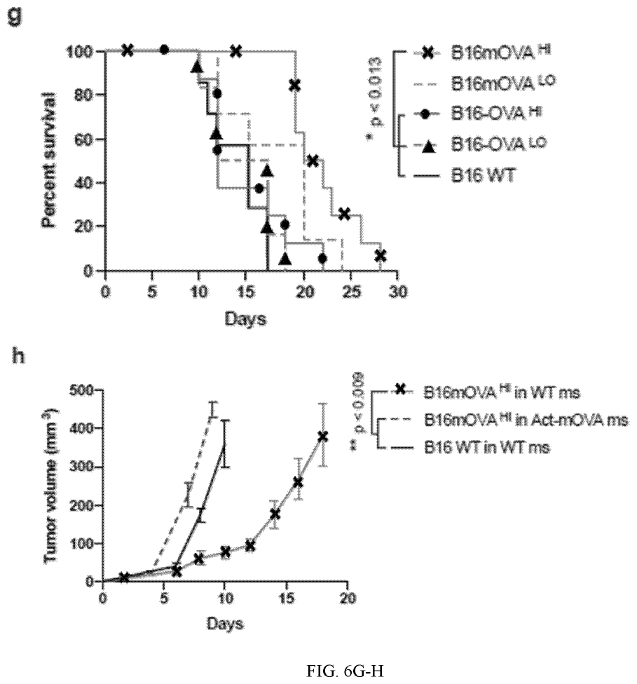

The inventors began by studying the effect of cell membrane-bound neoantigens in the B16-F10 murine melanoma model. The inventors first modified B16-F10 cells for expression of membrane-bound OVA (B16mOVA), by fusing the full-length OVA sequence to the transmembrane domain of H-2D^{B} (FIG. 6a) (21). As a control, the inventors used B16-F10 cells that expresses full-length OVA in a soluble form (i.e., not membrane-bound; B16-OVA). For both designs, the inventors generated cell lines with matching high (^{HI}) and low (^{LO}) levels of OVA expression, as quantified by qPCR (FIG. 6b, c). The presence of OVA at the surface of the B16mOVA cells, but not on the B16-OVA cells, was confirmed by flow cytometry and fluorescence (FIG. 6d, e). The inventors further highlighted that membrane-bound OVA was secreted on extracellular vesicles produced by B16mOVA (FIG. 6f), which is potentially important to increase antigen transport and availability to APCs.

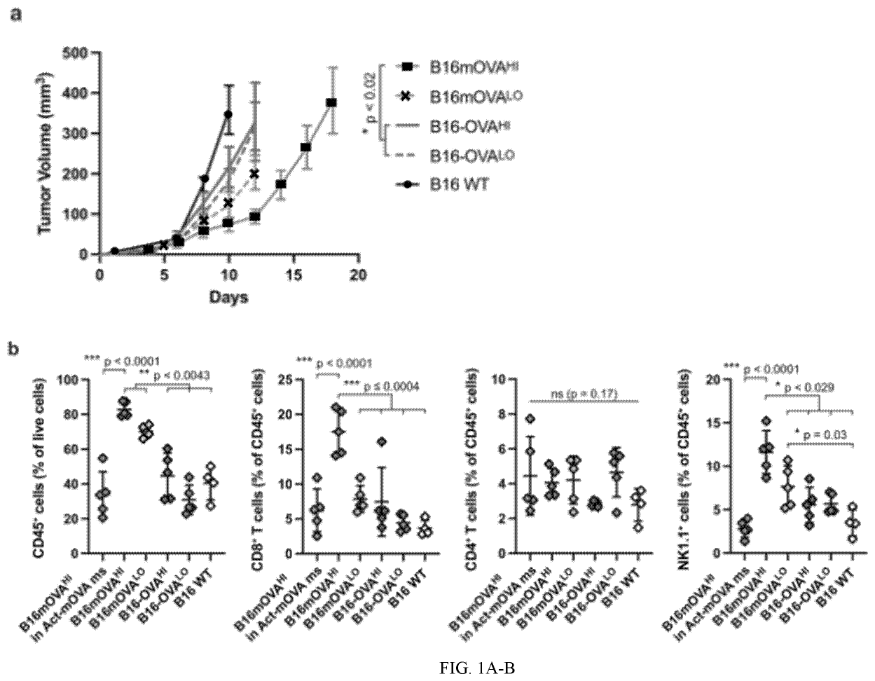

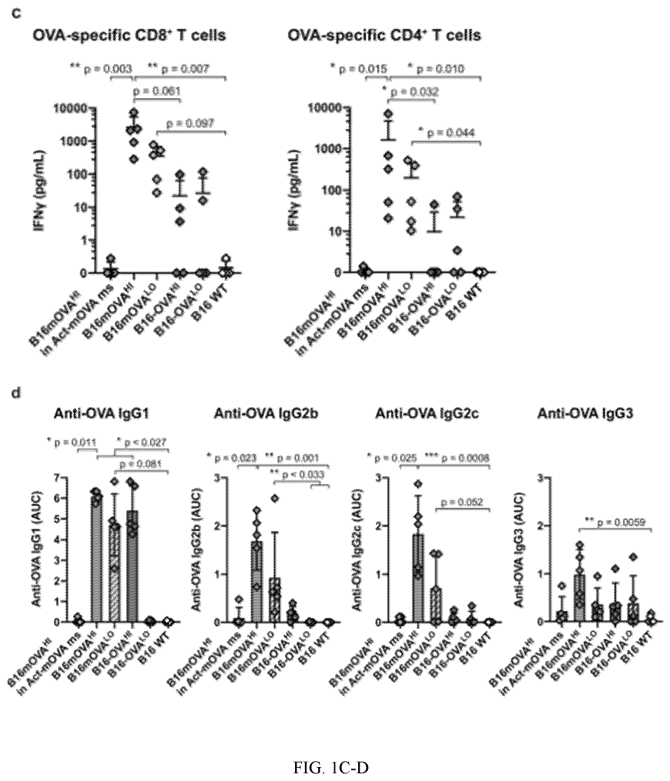

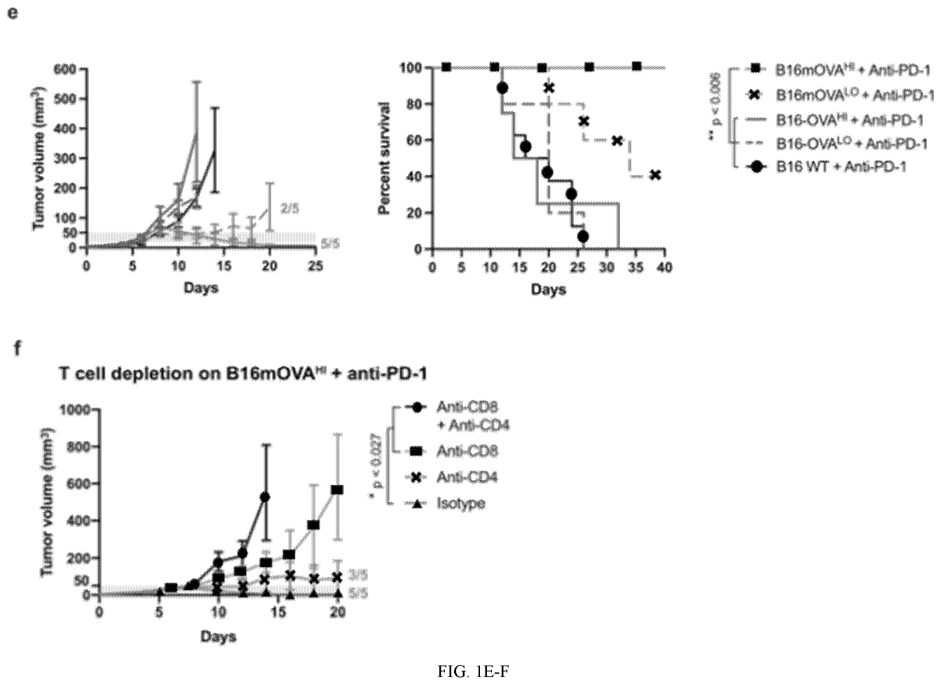

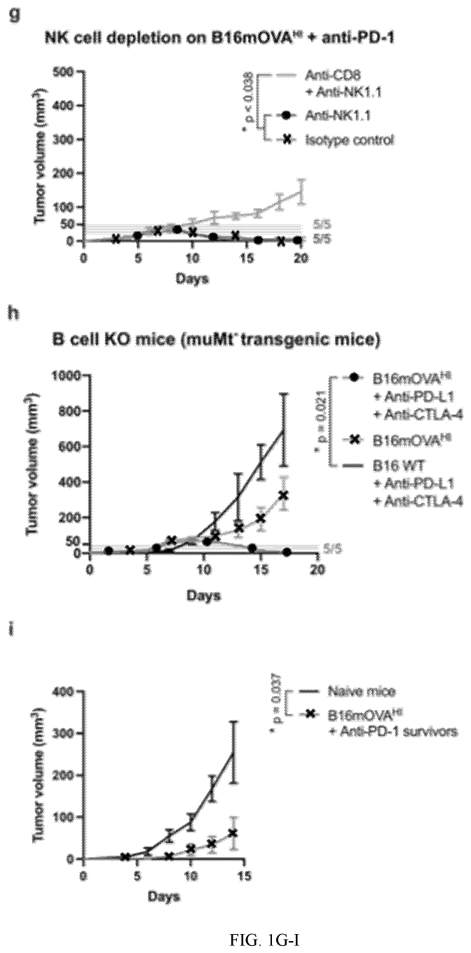

Upon intradermal injection in C57BL6 wild-type (WT) mice, all cell lines were tumorigenic. The inventors observed that B16mOVA^{HI} tumours grew significantly slower than B16-OVA^{HI} and the parental B16 WT, which resulted in extended survival of mice bearing B16mOVA^{HI} tumours (FIG. 1a, FIG. 6g). This effect was neoantigen dose-dependent, as seen by an intermediate growth rate of the B16mOVA^{LO} tumours. To confirm that this difference was due to immune-mediated rejection of the tumour, rather than to a difference in cell growth/division rate, the inventors evaluated B16mOVA^{HI} tumour growth in transgenic Act-mOVA mice, which are immune tolerant to mOVA. In these mice, B16mOVA^{HI} tumours grew faster than the B16 WT tumours, demonstrating an intact proliferation capacity of the B16mOVA^{HI} cells (FIG. 6h). This supports the hypothesis that the slowed tumour growth in WT mice was due to an immune reaction against mOVA.

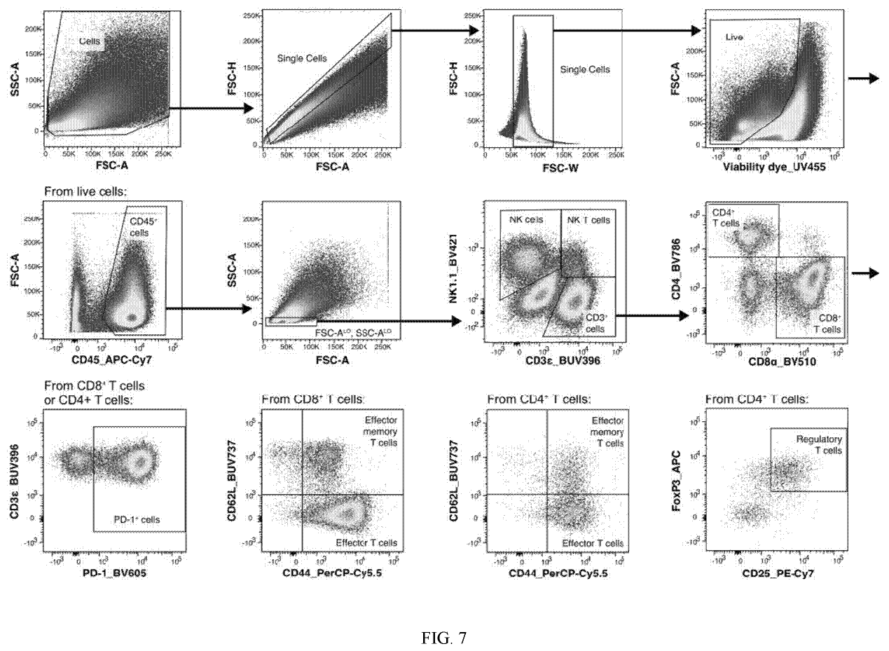

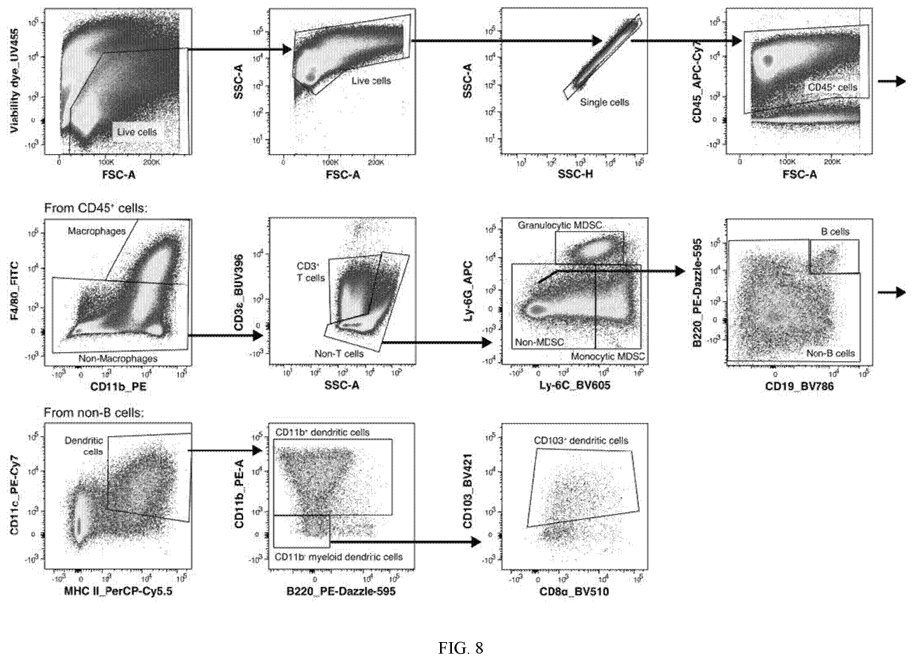

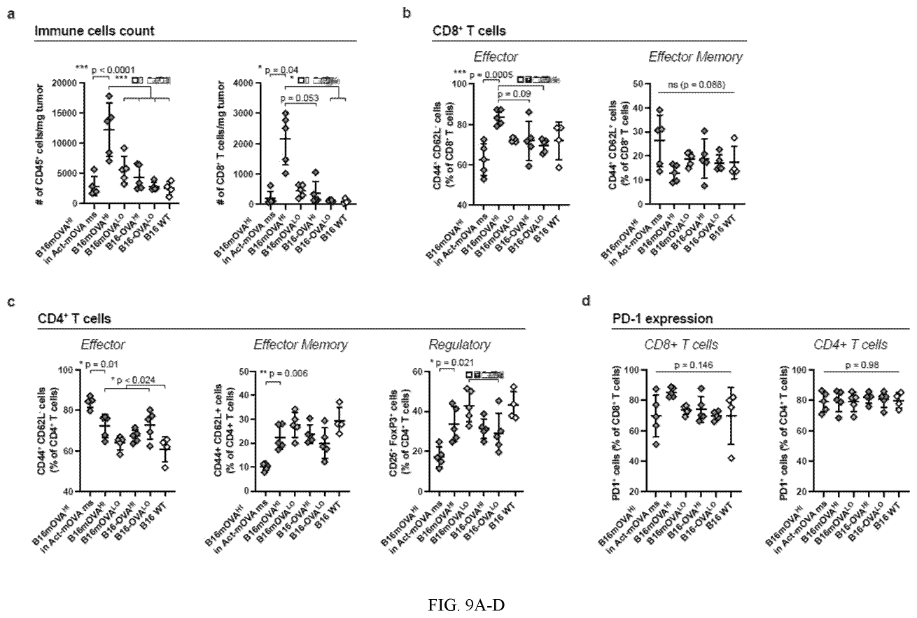

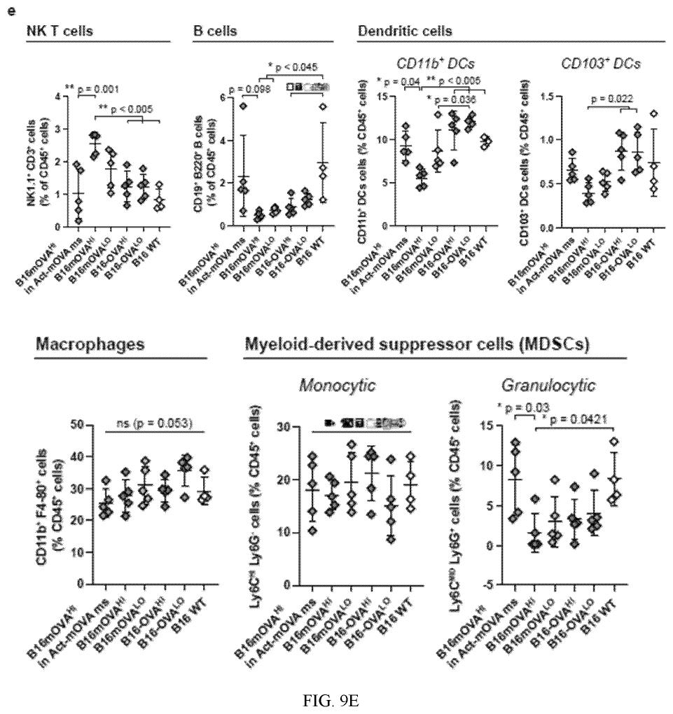

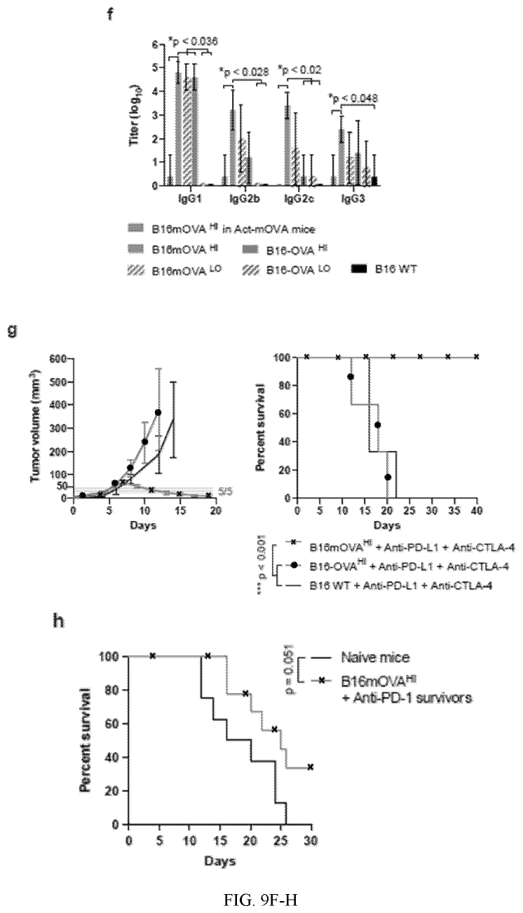

Therefore, the inventors analysed immune cell infiltrates in the different OVA-expressing B16-F10 tumours, reasoning that increased OVA-mediated tumour rejection would enhance the local presence of inflammatory cells (FIG. 7, 8). Indeed, the inventors found a significant increase of CD45⁺ immune cells in tumours that expressed mOVA as compared to dose-matched soluble OVA, of about 2-fold in the case of B16mOVA^{HI} vs. B16-OVA^{HI} tumours (FIG. 1b). Particularly, CD8⁺ T cells and NK cells were more numerous in this tumour, but not CD4⁺ T cells (FIG. 1b, FIG. 9a-c). No difference in PD-1 expression was observed on the T cells in the tumours expressing mOVA versus soluble OVA (FIG. 9d). Among the other immune cell types screened, NKT cells were slightly increased, and dendritic cells and B cells slightly decreased in the B16mOVA tumours when quantified relative to the total CD45⁺ immune cell population (FIG. 9e).

Next, the inventors assessed whether immunity against OVA in the B16mOVA-bearing mice was sufficiently strong to induce systemic immunity, in addition to local intratumoral inflammation. Ex vivo restimulation of splenocytes using OVA-derived MHC-I and MHC-II peptides revealed that both CD8⁺ and CD4⁺ T cell responses were increased in mice with tumour expressing mOVA as compared to those with soluble OVA, as highlighted by the production of the pro-inflammatory cytokine interferon (IFN)-γ (FIG. 1c). In addition, OVA-specific antibody responses were detected in the plasma of tumour-bearing mice for both B16mOVA and B16-OVA, but different subtypes of immunoglobulin G (IgG) were generated, depending on the antigen localisation. Particularly, OVA-specific IgG2b and IgG2c were detected in mice bearing B16mOVA tumours but were largely absent in those bearing B16-OVA tumours (FIG. 1d, FIG. 9f).

Together, these results showed that membrane-bound tumour cell neoantigens, here modelled by mOVA, strongly enhanced tumour immunogenicity both locally and systemically, resulting in slowed tumour growth and extended survival of untreated mice.

### 2. mAg Restore Responsiveness to ICI

While B16-F10 WT melanoma does not respond ICI, the inventors examined whether the increased immunogenicity of the B16mOVA, particularly the enhanced presence of intratumoral T cells, would render them more susceptible. Remarkably, all mice (5 out of 5) bearing B16mOVA^{HI} tumours and treated with anti-PD1 therapy showed complete responses to ICI, whereas B16-OVA^{HI} and B16 WT-bearing mice were completely unresponsive (FIG. 1e). Lowering the neoantigen dose in the B16mOVA^{LO} group reduced the efficacy of ICI yet resulted in 2 out of 5 tumour eradications and otherwise slowed tumour growth. Such effects were also confirmed using the combination therapy anti-PD-L1 and anti-CTLA-4 (FIG. 9g). In both therapies, responsiveness to ICI significantly extended survival.

The inventors then characterised which cell types were predominantly involved in the B16mOVA^{HI} tumour rejection by depleting specific immune cells populations upon ICI treatment. In the absence of the CD4⁺ T cells, CD8⁺ T cells were still capable of controlling tumour growth and led to the rejection in 3 out of 5 mice, thus with slightly lower efficacy than with proper help from the CD4⁺ T cells, as highlighted by the isotype control group in which all tumours were rejected (FIG. 1f). In contrast, CD4⁺ T cells alone were insufficient to eradicate tumours, although they slightly slowed tumour growth as compared to tumours depleted of both CD8⁺ and CD4⁺ T cells. Similarly, the inventors found that NK1.1⁺ cells were not required for responsiveness to ICI (FIG. 1g). Lastly, the inventors found that muMT⁻ transgenic mice, which lack mature B cells and cannot produce IgG, were able to reject B16mOVA^{HI} tumours upon ICI, suggesting that IgG-based antibody-dependent cytotoxicity mechanisms were not necessary for tumour eradication, although the inventors do not exclude that they might take place in WT mice (FIG. 1h).

Finally, the inventors investigated whether the immune rejection of the B16mOVA tumours upon ICI was solely directed against membrane-bound OVA or if immune reactions against other tumour-associated antigens were at play. Upon re-challenge, mice that rejected B16mOVA tumours showed delayed growth of B16 WT tumours, suggesting the presence of pre-existing immune reactions against B16 WT neoantigens induced during the initial rejection of B16mOVA (FIG. 1i, FIG. 9h). Of note, the secondary B16 WT tumours remained non-responsive to ICI. Therefore, while mOVA was necessary to eradicate the primary tumour upon ICI, its loss in the secondary tumours still resulted in delayed tumour growth, potentially mimicking a situation of cancer relapse or metastasis.

### 3. mAg Increase Patient Survival Upon ICI

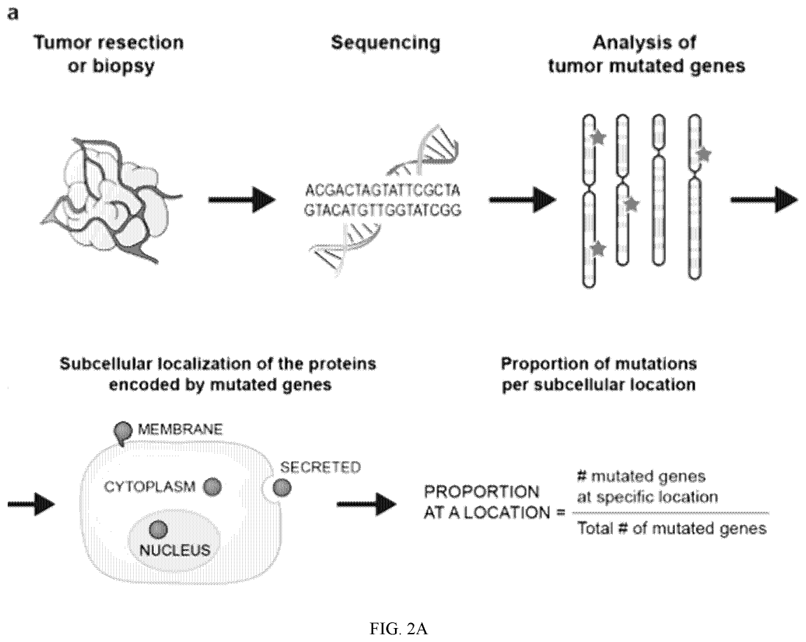

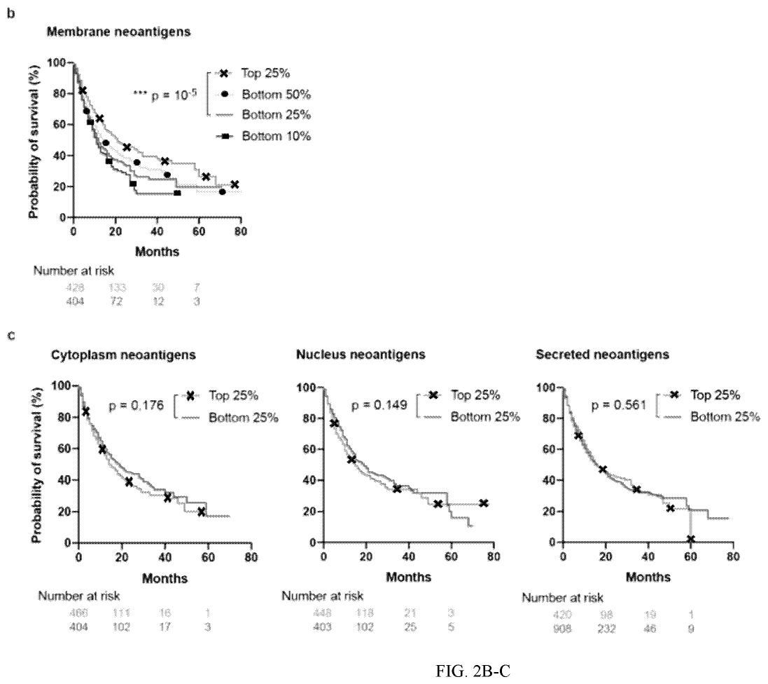

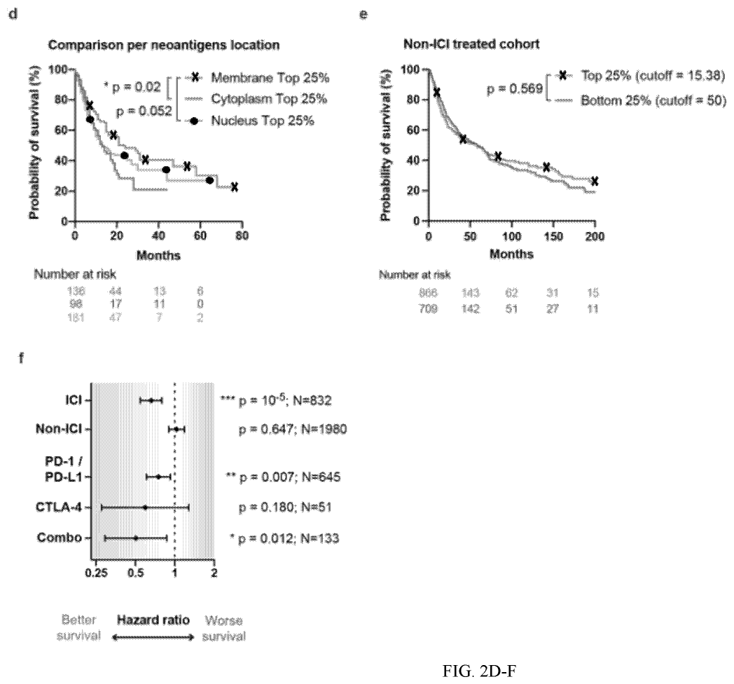

The remarkable ability of membrane-bound neoantigens to restore responsiveness to ICI in the murine melanoma model encouraged us to validate this hypothesis in cancer patients. Therefore, the inventors analysed publicly available tumour mutation sequencing data of patients treated or not with ICI, from 3 independent studies by Samstein et al. (7), Hellman et al. (8) and Hugo et al.⁹. For each tumour mutated gene detected in patients, the inventors extracted the subcellular localisation of its encoded protein from the UniProtKB/Swiss-Prot database (20) (Supplementary Data 1). The inventors then quantified per patient the number of mutated genes that encode for membrane, cytoplasmic, nuclear, or secreted proteins. Genes that encode proteins expressed at several localisations were classified in all locations, in a non-exclusive manner. The inventors lastly normalised the number of neoantigens at a specific subcellular location to the total number of mutated genes, therefore obtaining proportions of neoantigens per subcellular location (FIG. 2a).

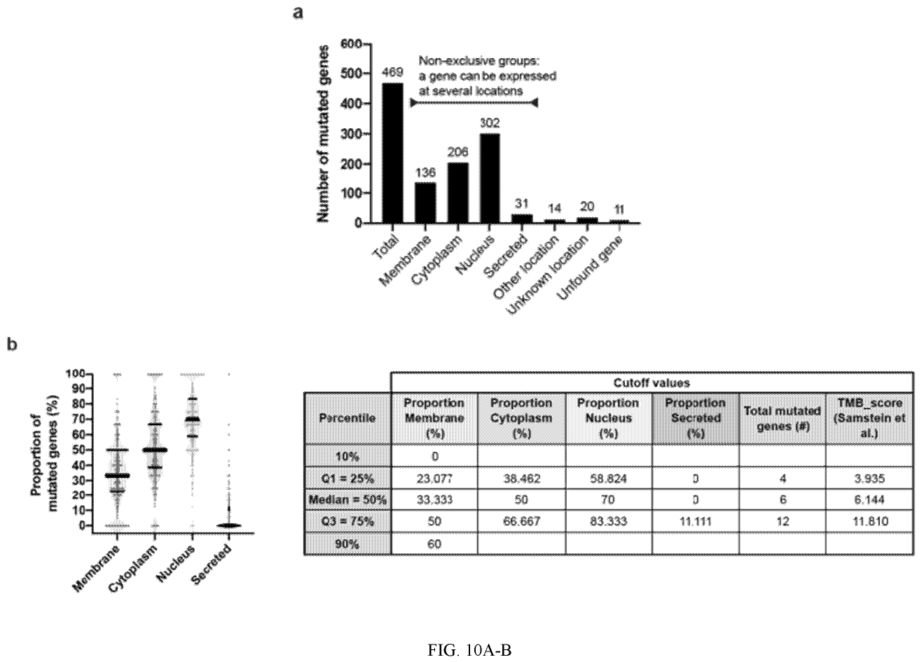

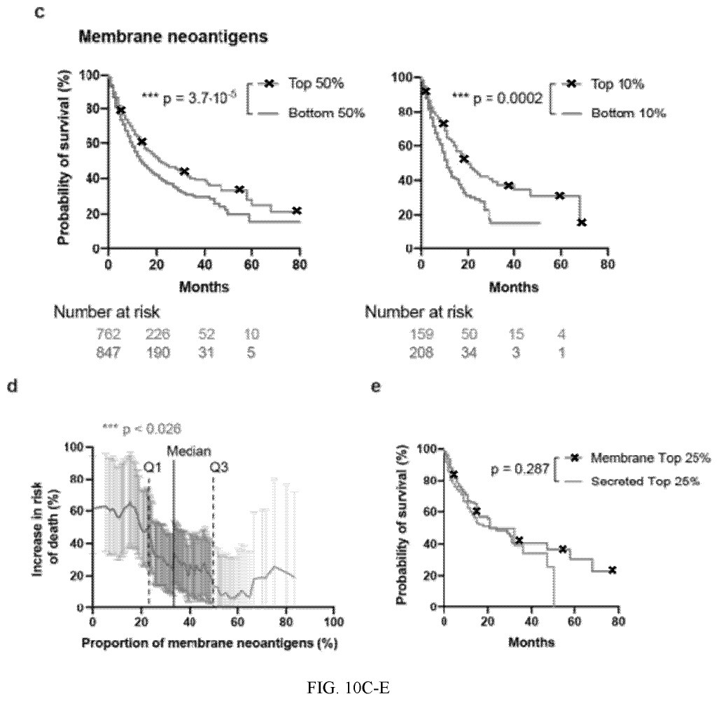

The inventors first analysed the dataset by Samstein et al. (7) comprising of 1609 patients with 9 different types of advanced cancers treated with ICI whose tumour mutations were determined using targeted next-generation sequencing MSK-IMPACT (Suppementary Data 2). In total, 424 genes out of the 469 sequenced were classified in the 4 subcellular locations of interest (FIG. 10a). The inventors compared groups of patients with high and low proportion of neoantigens for each specific location using the cutoff values of the upper and bottom group quartiles (Top 25% vs. Bottom 25%; FIG. 10b). A high proportion of mAg was found to correlate with significantly increased patient survival (FIG. 2b). This effect also was conserved at other percentiles than 25% (FIG. 10c). Interestingly, an insufficient proportion of mAg was strongly associated with worsened survival, as highlighted by the gradual decrease between the groups Bottom 50%, 25% and 10%, with the Bottom 10% group being patients with no mAg (FIG. 2b, FIG. 10d). None of the other subcellular locations correlated with significant improvement in survival (FIG. 2c). Instead, trends toward reduced survival were observed for cytoplasmic and nuclear neoantigens, and no difference was seen for secreted neoantigens. Further division into exclusive patient groups with high proportions of neoantigens at a single location highlighted that the membrane neoantigen localisation provides higher survival benefits than the cytoplasmic and nuclear localisations (FIG. 2d, FIG. 10e).

The inventors then questioned whether the survival advantage that correlated with a high proportion of mAg was present in non-ICI treated patients. The inventors analysed 3142 patients from the non-ICI treated control cohort of Samstein et al. (7,22) (Supplementary Data 3) and found that no survival benefit was associated with membrane localisation in absence of ICI (FIG. 2e, f). However, all types of ICI therapies, namely PD-1/PD-L1, CTLA-4 or the combination PD-1/PD-L1⁺CTLA-4, correlated with extended survival in patients harbouring a high proportion of mAg, as indicated by a hazard ratio (HR) for survival inferior to 1. This effect did not reach statistical significance for CTLA-4, likely due to the limited number of patients in this group (FIG. 2f).

Together, these findings suggest that high proportions of tumour mAg improve cancer patient survival upon different types of ICI treatments.

### 4. Impact of mAg in Different Cancer Types

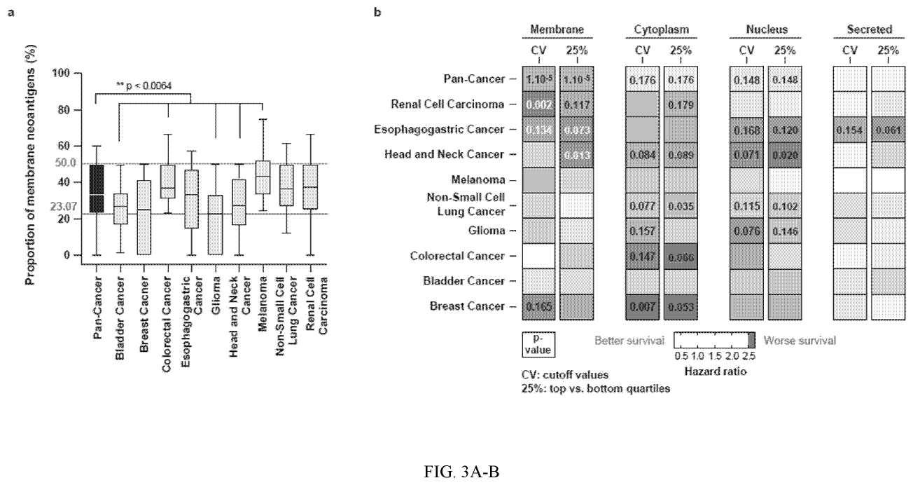

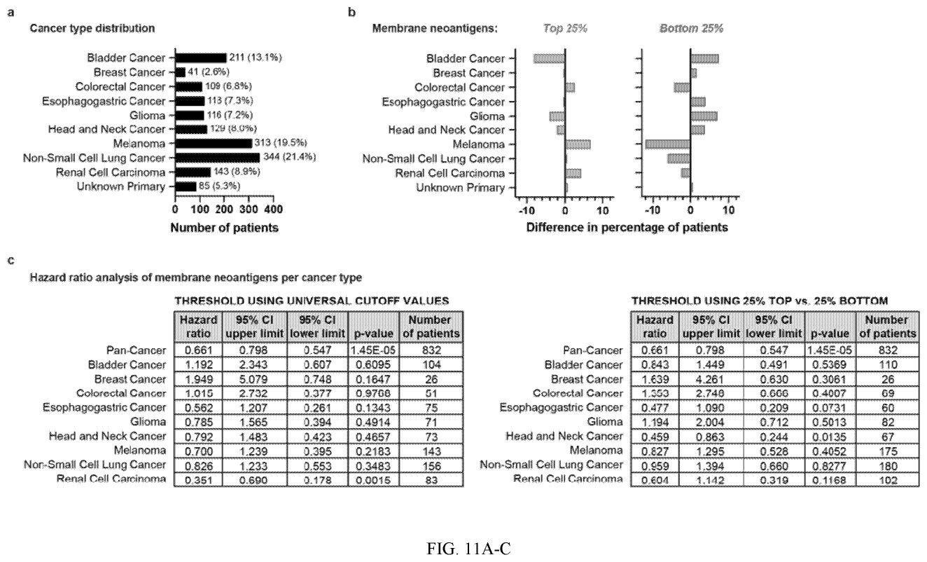

The ICI-treated cohort analysed above included patients with 9 different types of cancers, non-equally distributed (FIG. 11a). When comparing the distribution of cancer types within the Top 25% and Bottom 25% of the membrane-localised neoantigen groups, the inventors noticed that the population with high mAg proportion was enriched in melanoma, renal cell carcinoma and colorectal cancer patients, and depleted from bladder cancer, glioma and head-and-neck cancer patients (FIG. 11b). This implied that not all cancer types had the same distribution of mAg proportion; in fact, glioma, bladder and head-and-neck cancers had significantly less mAg than the pan-cancer group, whereas colorectal and melanoma cancers had significantly more (FIG. 3a).

Therefore, the inventors detailed the effects of high mAg burden, as well as of other subcellular localisations, per cancer type. The inventors computed the HR for survival to compare patients with high versus low proportions of neoantigens at a specific location, using 2 different strategies: 1) keeping the same cutoff values that the inventors used for the pan-cancer group analysis in FIG. 2, reasoning that a “universal” threshold might be determined across cancers as being an absolute proportion of neoantigens required for extended survival, or 2) using the upper and lower quartile values specific to each cancer type (FIG. 3b, FIG. 11c). Overall, a high proportion of mAg correlates with better survival in 6 out of 9 individual cancers, with statistical significance reached in the renal cell carcinoma and head-and-neck cancer, and close to significance for esophagogastric cancer. The lack of significance in the other cancer types might be due to smaller effects or limited numbers of patients in each sub-cohort. On the other hand, cytoplasmic and nuclear neoantigens were associated with worsened survival in a majority of cancer types (6 out of 9; 1 or 2 significantly). Secreted neoantigens did not strongly impact patient survival, except in the esophagogastric cancer, in which a trend toward improvement was observed. Interestingly, both thresholding methods for the selection of high vs. low groups showed very similar results, except for glioma and bladder cancers at the membrane locations. Further analysis with a higher number of patients would clarify whether an absolute threshold for proportion of mAg can be determined to predict increased survival upon ICI across cancers.

### 5. mAg Predict Patient Response to ICI

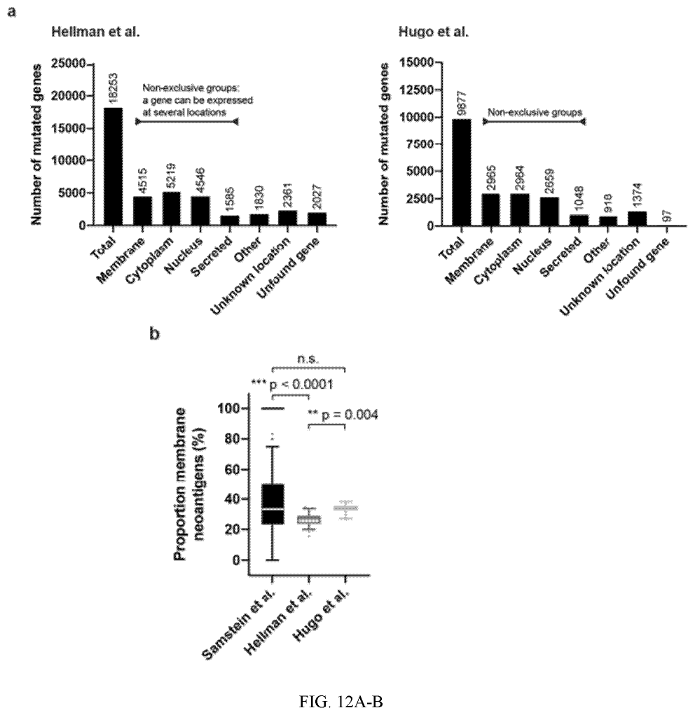

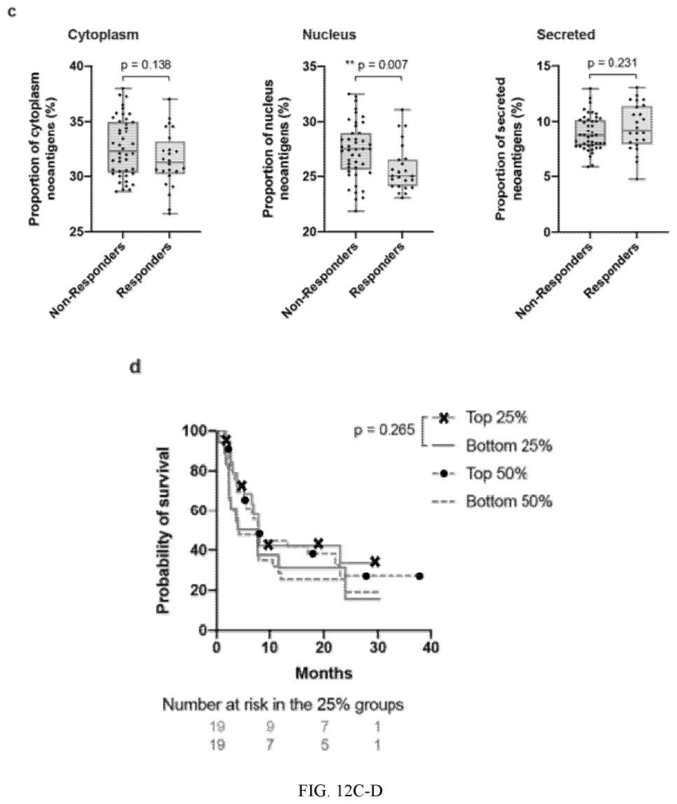

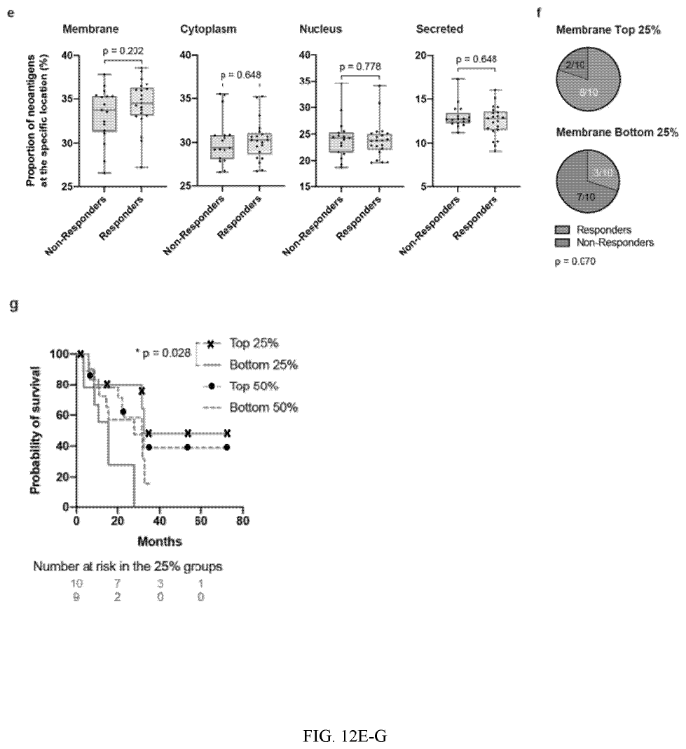

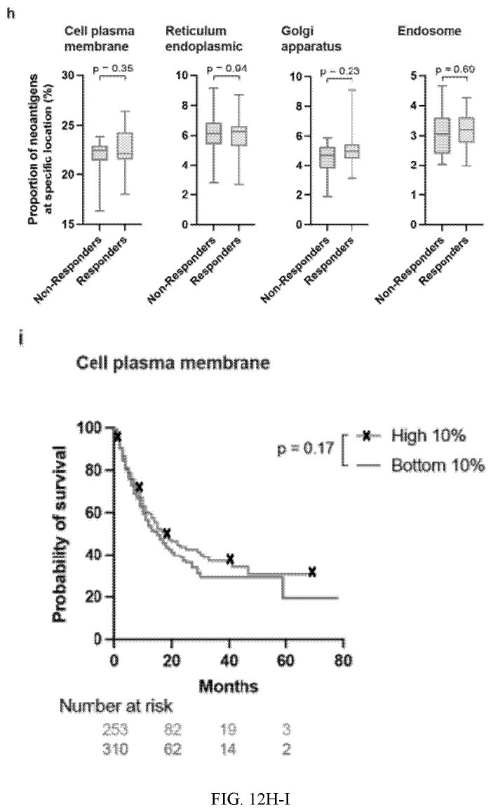

While the metric of survival is a relevant measure to evaluate effectiveness of ICI, response rate and long-term survival do not always correlate well. Hence, the inventors searched for published datasets in which the patient response to ICI was reported. The inventors found 2 such studies, from Hellman et al. (8) and Hugo et al. (9), which respectively focused on ICI-treated patients with NSCLC (75 patients) and metastatic melanoma (38 patients). Both studies used whole-exome sequencing (WES) to determine tumour mutations in patients treated with anti-PD-1 or with the combination of PD-1 and CTLA-4 blockade. The inventors thus repeated the neoantigen subcellular localisation analysis using the same algorithm to categorise tumour mutations according to their possible expression in the membrane, cytoplasm, nucleus or secreted category (FIG. 12a, Supplementary Data 4, 5). Because more genes were sequenced by WES than by MSK-IMPACT, the detected variation range of membrane neoantigen proportion in the WES-sequenced patients was much smaller, with most patients having between 25-35% of membrane mutations. Interestingly, the overall median proportion of membrane neoantigens remained similar between the studies, with 33.3%, 27.0% and 34.3% in Samstein et al. (7), Hellman et al. (8) and Hugo et al. (9), respectively (FIG. 12b). The small difference of lowered membrane proportion found in the cohort from Hellman et al. (8) might be due to the increased number of genes for which the subcellular locations could not be determined.

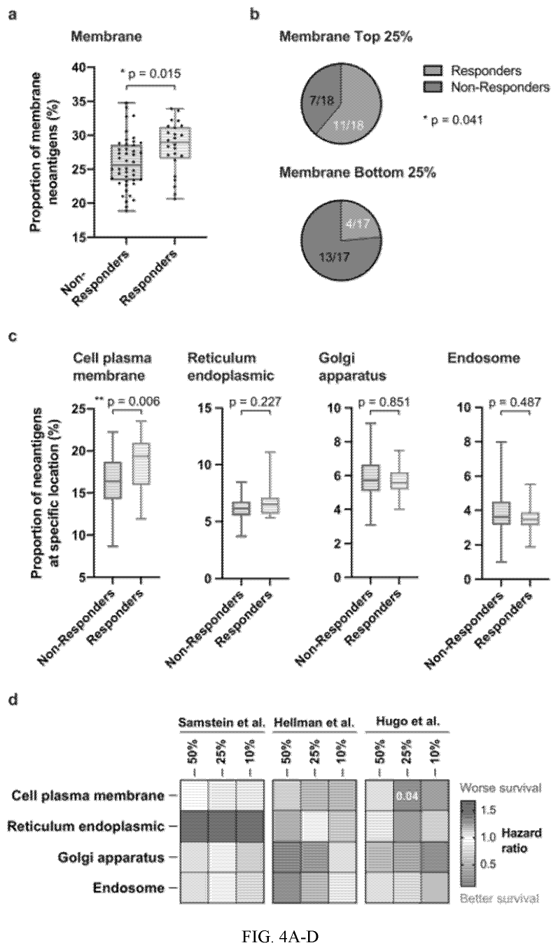

In this NSCLC cohort (8), patients that responded to ICI had a significantly higher proportion of neoantigens at the membrane, but not at the other studied locations. In addition, these patients tended to survive longer, although statistical significance was not obtained, highlighting the potential discrepancy between ICI responsiveness and overall survival readouts (FIG. 4a, FIG. 12c, d). Impressively, the response rate was 61% in the group with high mAg proportion (25% Top), vs. 23.5% in patients with low mAg proportion (25% Bottom) (FIG. 4b). Similar trends were also observed in the melanoma cohort, despite the low number of patients (FIG. 12e-g). Thus, these two additional studies further support the hypothesis that a high proportion of membrane-localised neoantigens correlates with ICI responsiveness, consistently with the survival results obtained in the larger, multi-cancer cohort from Samstein et al.⁷. Importantly, they also point out that this effect was conserved independently of the sequencing methods used for the detection of the tumour mutations.

### 6. Neoantigens of the Plasma Membrane

Observing that membrane-localised neoantigens lead to greater response to ICI, the inventors questioned whether there were differences between particular membranes in the cell. To address this, the inventors refined the algorithm to segregate for cell membrane (i.e., plasma membrane), endoplasmic reticulum, Golgi apparatus or endosome localisations. Using the data on ICI responders from the NSCLC cohort (8), the inventors found that only the proportion of neoantigens at the cell plasma membrane was significantly increased in ICI responders, while localisation at the membranes of organelles did not correlate with changes in ICI response (FIG. 4c). Similar trends were observed for the melanoma cohort (FIG. 12h). In addition, consistent trends toward improvement of survival for patient with increased proportion of cell plasma membrane neoantigens was observed across the pan-cancer, NSCLC and melanoma cohorts (FIG. 4d, FIG. 12i).

### 7. mAg as Clinical Biomarkers for ICI

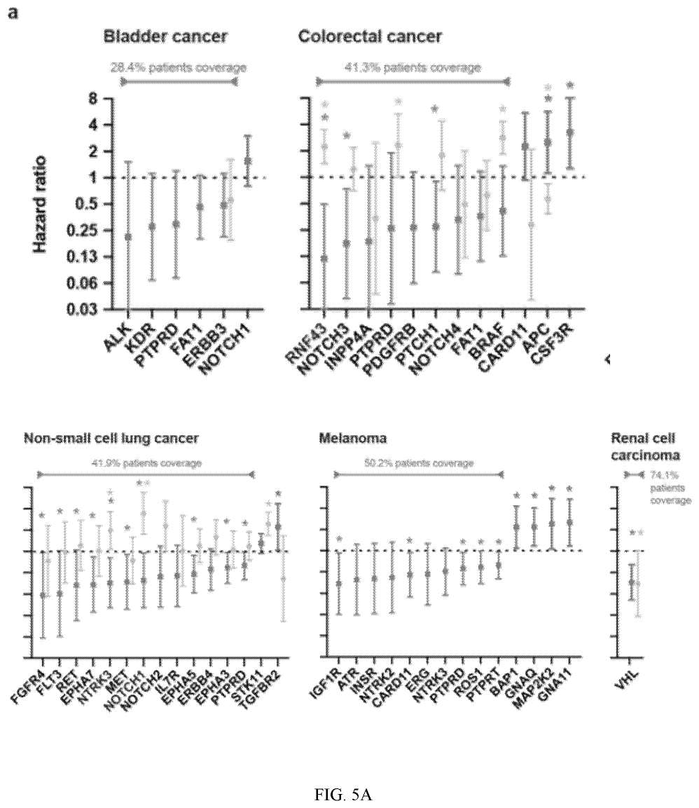

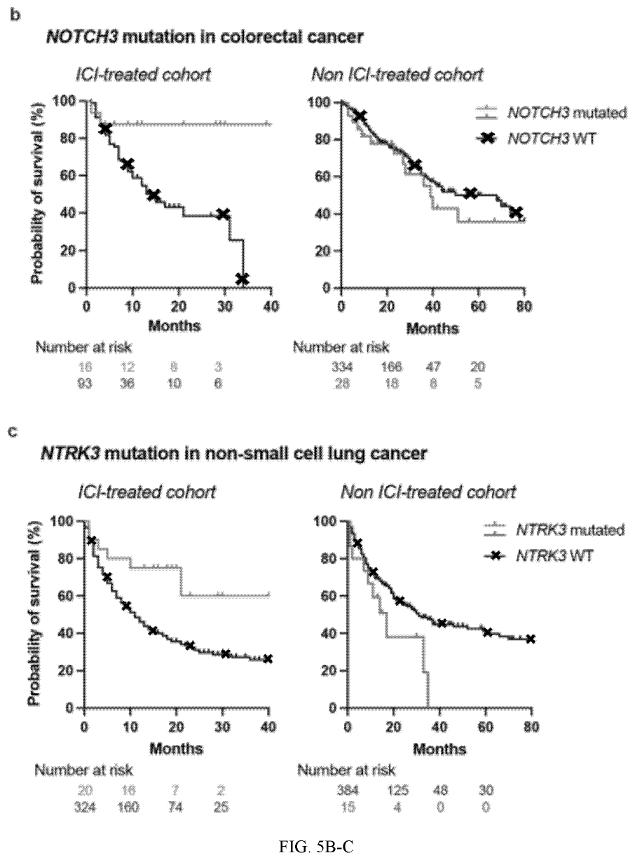

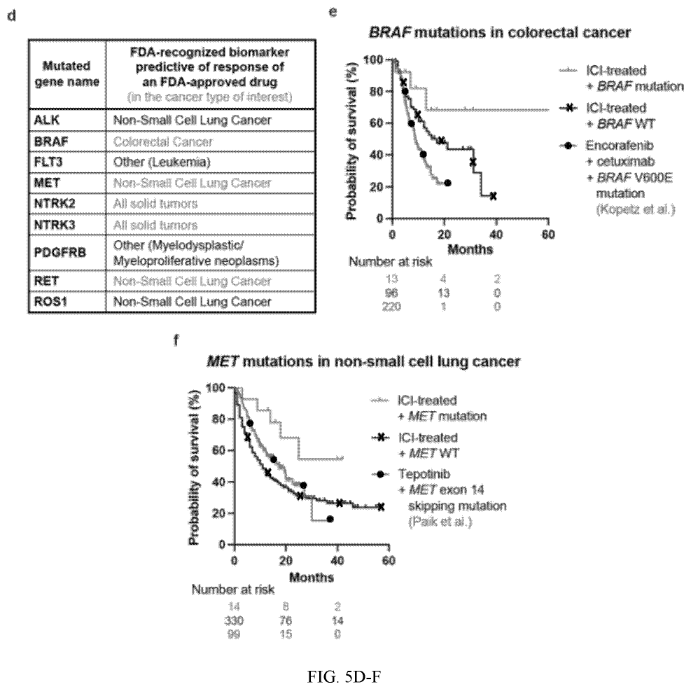

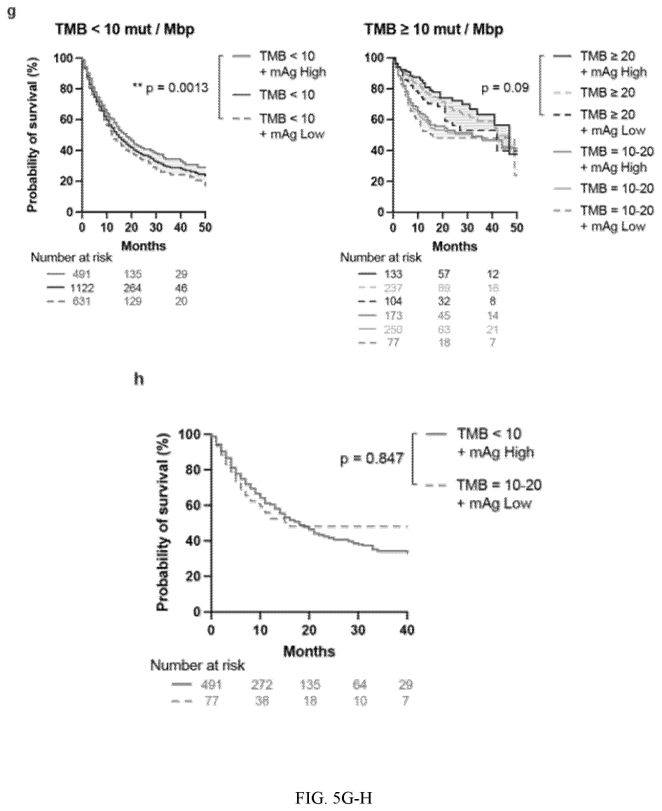

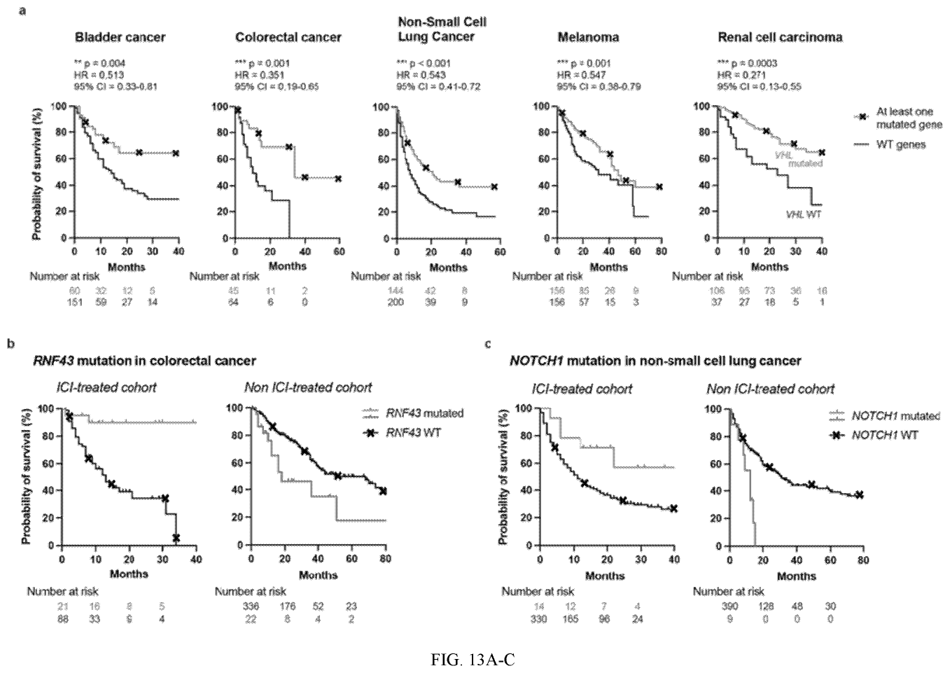

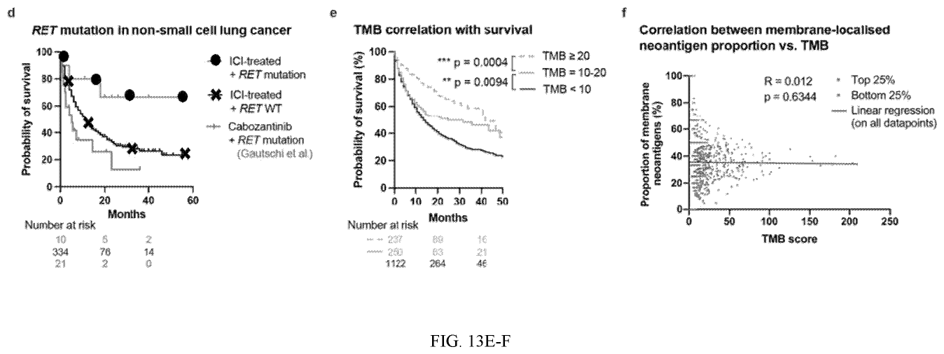

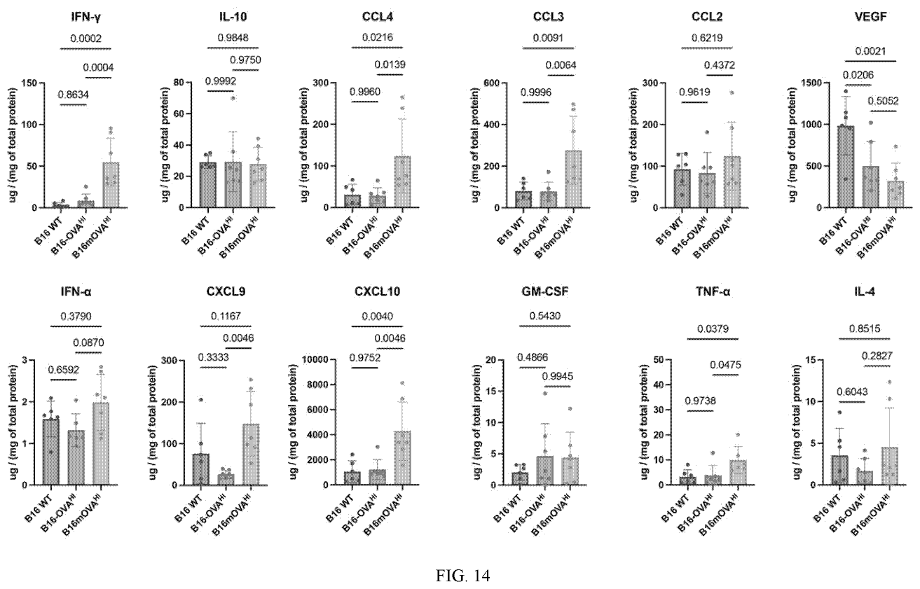

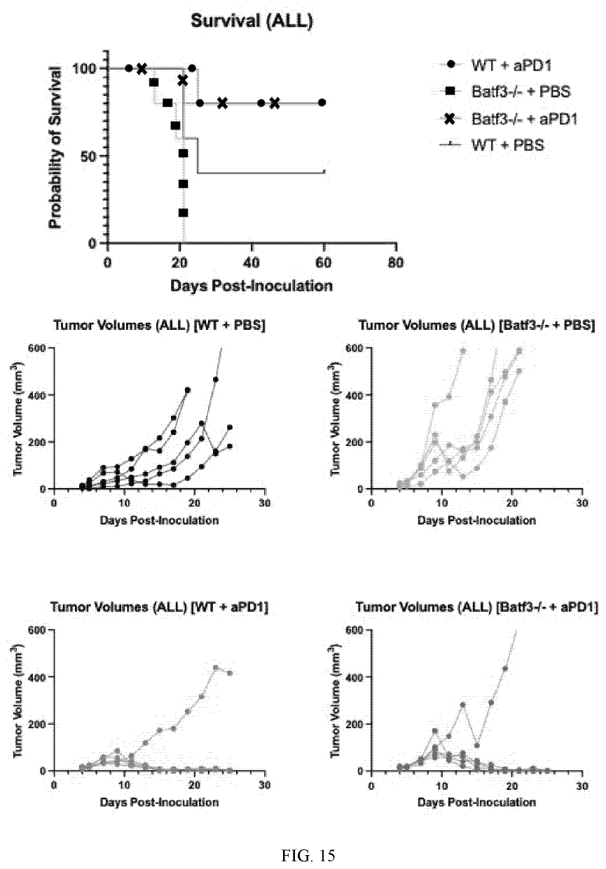

Finally, the inventors analysed which mAg most impact survival upon ICI. Using the dataset from Samstein et al. (7), the inventors computed the HR of survival between patients bearing mutated and wild-type membrane protein-encoding genes, within each cancer type (FIG. 5a, Supplementary Data 6). The inventors observed that most of the mutated genes correlated with improved survival, although a few of them correlated with worsened survival. The inventors particularly highlighted a subset of 1-13 genes per cancer type for which mutations could serve as potent biomarkers to predict extended survival upon ICI, as indicated by low HRs. Interestingly, the inventors found that patients bearing at least one of these biomarkers survived significantly longer than patients with none, in all the cancer types for which enough patients were available, i.e., bladder cancer, colorectal cancer, NSCLC, melanoma and renal cell (FIG. 13a). This represents a substantial proportion of patients, between 28.4% and 74.1% depending on the cancer type, thus highlighting a strong potential for clinical translation of these membrane-localised biomarkers sets.

Further seeking ICI-specific membrane-localised biomarkers, the inventors compared the HRs obtained upon ICI to the ones from the non-ICI-treated cohort, for each gene for which enough patients were available (FIG. 5a, Supplementary Data 6). In most cases, gene mutations did not seem to improve survival in the non-ICI-treated cohort to the same extent than in the ICI-treated cohort, suggesting that these biomarkers could be specific for prediction of ICI efficacy. One exception was VHL in renal cell carcinoma, for which mutations appeared to be beneficial in both cohorts. On the other hand, the inventors found that some mutated genes correlated with very high survival in the ICI-treated cohort, but with worsened survival in the non-ICI-treated one, such as NOTCH3 or RNF43 in colorectal cancer (the latter having been recently elucidated by Zhang et al. (23)), and NTRK3 and NOTCH1 in NSCLC (FIG. 5b, c, FIG. 13b, c). Upon confirmation by future studies, such genes could constitute very promising stand-alone biomarkers to guide medical choice toward ICI rather than other treatments in specific cancer types.

Among the membrane-localised biomarkers that the inventors highlighted, a few are recognized by the U.S. Food and Drug Administration (FDA) as biomarkers predictive of response of a FDA-approved drugs (24) (FIG. 5d), and thus are currently assayed in the clinic. It is the case for some BRAF mutations in colorectal cancer, MET and RET mutations in NSCLC, or NTRK2/3 mutations (24). Therefore, the inventors attempted to compare the performance of these membrane-localised biomarkers to predict survival upon ICI versus upon their clinically-associated treatments, using survival data from other published clinical studies by Kopetz et al. (25), Paik et al. (26) and Gautschi et al. (27). While such direct comparisons can not be conclusive due to intrinsic differences in the study designs, BRAF and RET mutations seem highly effective as biomarkers for ICI as compared to the FDA-approved encoferanib+cetuximab in colorectal cancer (25) (FIG. 5e) and tepotinib in NSCLC (26) (FIG. 5f), respectively. A similar observation was made for RET mutations, when compared to a standard-of-care treatment with cabozantinib in NSCLC (27) (FIG. 13d).

Last but not least, the FDA has very recently approved the use of high TMB (i.e., TMB≥10 mutations/megabase pair (mut/Mbp)) as a criterion for ICI, for adults and children with unresectable or metastatic solid tumours that failed to respond to prior therapies (28), thus fostering the use of next-generation sequencing of tumour mutations in the clinic. Because determination of the proportion of tumour mAg from these sequencing data would require only a simple algorithm but no additional clinical or laboratory procedures, the inventors examined the benefit of combining the mAg proportion with the TMB to predict survival upon ICI. The inventors found that a high proportion of mAg correlated with improved survival in patients with both low TMB (<10 mut/Mbp) or high TMB (≥10 mut/Mbp) (FIG. 5g, FIG. 13e, f). In addition, the inventors observed that some patients with high TMB (10-20 mut/Mbp) but low mAg, for which ICI is approved, had similar survival as patients with low TMB but high mAg (FIG. 5h), which may not currently qualify for ICI. Importantly, the latter represent 30.5% of the patients in the Samstein et al. dataset, which could thus be considered for ICI but would not be otherwise. Together, this suggests that the proportion of mAg could be a valuable parameter to take into account, on top of the TMB, to extend the inclusion criteria for ICI in the clinic.

### B. Discussion

This study focused on the influence of the subcellular localisation of tumour neoantigens for responsiveness to cancer immunotherapy. Importantly, the inventors demonstrated in both the B16-F10 melanoma mouse model and on a large clinical dataset of 4864 ICI- and non-ICI-treated cancer patients that responsiveness to ICI and extended survival correlated with a high proportion of membrane, especially plasma membrane, neoantigens. Interestingly, this effect was not seen for increased load of cytoplasmic, nuclear or secreted neoantigens, nor was it seen in patients that were not treated with ICI. Nevertheless, in-depth analysis per cancer type would be needed on larger number of patients to further elaborate on these conclusions, as the inventors pointed out that a high proportion of mAg might have varying effects in different cancer types.

In the analysis, the inventors found consistent results from clinical datasets published by three independent research groups, which used two different methods of tumour mutation sequencing, namely MSK-IMPACT and WES, both recently approved by the FDA and rapidly emerging in the clinic (29-32). While these sequencing methods aim to quantify TMB, high load of which is approved as an inclusion criterion for treatment with ICI, this work provides a complementary simple algorithm-based method that can further filter the sequencing data to improve the prediction accuracy of ICI responsiveness. It was found that the mAg criterion indicates 30.5% more patients for inclusion into ICI than the current FDA standard of TMB based on the Samstein et al. dataset (7). In addition, the inventors highlighted particular mAg that may be very potent stand-alone predictive biomarkers to guide the choice toward treatment by ICI in certain cancer types.

Besides the basic immunology perspective, this work provides a rationale for therapeutic immunomodulation by neoantigen selection at different subcellular locations. In particular, personalised cancer vaccines currently target neoantigens based on prediction of MHC binding neoepitopes for optimised T cell activation, however with little consideration of the subcellular localisation of the neoantigen (13). Adding antigen selection criteria for preferential targeting of plasma membrane neoantigens might improve the therapeutic efficacy of such vaccines. Taken together, the inventors believe that the simplicity of considering the neoantigens' subcellular localisations for increased predictability to ICI response, the use of mAg as biomarkers to guide medical decisions of cancer treatments, as well as the possible impacts on the design of future immunotherapies, will be valuable in the fight against cancer.

### C. Methods

### 1. OVA-Expressing B16 Melanoma Cell Lines

B16F10 (B16) melanoma cells (American Type Culture Collection, Manassas, VA, USA) were genetically modified by transduction with OVA-encoding lentivirus. Briefly, OVA-encoding DNA sequences were purchased from GenScript (Piscataway, NJ, USA). In one design, full-length OVA (UniprotKB P01012) was fused at the N-terminus to the signal peptide of mouse H-2K^{B} (aa1-aa21, UniprotKB P01901) and at the C-terminus to the transmembrane domain of mouse H-2DB (aa299-aa331, UniProtKB P01899). Sequences were subcloned in the pLV-mCherry backbone (Addgene #36804) in place of mCherry. Lentiviruses were made by polyethylenimine (PEI)-mediated transfection of human embryonic kidney (HEK) 293-T cells using OVA-encoding plasmid with the packaging plasmids pMD2.G (Addgene #12259), pMDLg/pRRE (Addgene #12251) and pRSV-Rev (Addgene #12253). Twelve hours after transfection, the cell culture medium was refreshed and 36 h later, the medium was collected and filtered at 0.22 μm. Lentiviruses were concentrated by ultracentrifugation at 100,000×g for 2 h at 4° C. and resuspended in phosphate-buffered saline (PBS). B16 cells cultured in 48-well plates were transduced by adding OVA-encoding lentiviruses in the culture medium and centrifuging at 1150 xg for 30 min at room temperature, and then were cultured for 24 h, after which the medium was refreshed. For B16mOVA^{HI/LO} and B16-OVA^{HI}, monoclonal selection was performed by limiting dilution, and OVA-expression was quantified by quantitative polymerase chain-reaction (qPCR). The B16-OVA^{LO} cell line was a gift from B. Huard (University of Geneva, Switzerland). All cell lines were tested as negative for mycoplasma contamination by PCR.

### 2. Quantitative PCR for OVA Expression

Expression of OVA in B16 cell lines or tumours was quantified by qPCR. Prior to RNA extraction, 30-50 mg of tumour tissues were homogenised (FastPrep-24 5G, MP Biomedicals, Santa Ana, CA, USA) in RLT lysis buffer (Qiagen, Hilden, Germany), spun down at 10,000 xg for 10 min and the supernatant was collected. For cells in culture, 1-2 million cells were pelleted, washed with PBS and lysed in RLT buffer. RNA was extracted using the RNeasy Plus Mini kit (Qiagen). The extracted RNA (1 μg) was then converted to cDNA using SuperScript IV VILO Master Mix (ThermoFisher Scientific, Waltham, MA, USA). All kits were used according to manufacturers instructions. TaqMan qPCR were finally performed using TaqMan Universal PCR Master Mix, OVAL primer (Gg03366807_ml) and ActB primer (Mm02619580_g1) (ThermoFisher Scientific), in a LightCycler 96 real-time PCR system (Roche Life Science, Basel, Switzerland).

### 3. Detection of Membrane-Bound OVA

Surface-expression of OVA was verified by flow cytometry and microscopy. Single cell suspensions of the different OVA-expressing B16 were incubated for 30 min on ice with anti-OVA (ab181688, Abcam, Cambridge, UK) in PBS+2% foetal bovine serum (FBS). Cells were washed twice and stained using an anti-rabbit secondary antibody (A315723, Invitrogen, Carlsbad, CA, USA) for 20 min on ice in the dark. Cells were washed and analysed by flow cytometry (BD LSRFortessa, BD Biosciences, Franklin Lakes, NJ, USA) or imaged by fluorescence microscopy (Leica DMi8, Wetzlar, Germany). Flow cytometry data were analysed using FlowJo (FlowJo LLC) and microscopy images were processed using Fiji (ImageJ, U.S. National Institutes of Health, Bethesda, MD, USA).

### 4. Extracellular Vesicles (EV) Isolation

EV from the B16mOVA^{HI} and B16-OVA^{HI} cell lines were harvested using the CLAD1000 system (2440655, Cole-Parmer, Vernon Hills, IL, USA) as described by Mitchell et al. (37). Briefly, 16 million cells were suspended in 15 mL complete EV-depleted DMEM (DMEM+1% penicillin/streptomycin (P/S)+10% exosome-depleted FBS (A2720801, Thermo Fisher Scientific)) and loaded into the lower chamber of the CLAD flask. The upper chamber was then loaded with DMEM+1% P/S, and cells were allowed to recover for 4 days. On the 4th day, the upper reservoir was emptied and the media in the lower chamber was collected. The lower chamber was washed twice with DMEM, collecting only the first wash. The lower chamber was then refilled with 15 mL of complete EV-depleted DMEM. This harvesting process was repeated every 4 days. Collected media was first spun at 300×g for 10 min to remove cells, then centrifuged at 3000×g for 10 min to remove large cell debris and finally at 10,000 xg for 30 min to further remove debris. The final supernatant was concentrated using 100,000 MWCO concentrator tubes (UFC910024, EMD Millipore, Burlington, MA, USA) before processing via size exclusion. Size exclusion was performed using the Izon qEV10 system (IZON SP3) according to the manufacturer's instructions to collect separately the EV fractions, containing particulates of 70-1000 nm in size, and the non-particulates non-EV fractions. Once purified, EV harvests were pooled and re-concentrated. Total protein content of the purified EV was quantified using a Micro BCA kit (Thermo Fisher) before storage at −20° C. Equal amount of proteins (34 μg) were loaded on SDS-PAGE gels for further analysis by western blot.

### 5. Western Blot Analysis

Samples were run on SDS-PAGE gels for 45 min at 140 V (Mini-PROTEAN gel system, Bio-Rad Laboratories, Hercules, CA, USA) in Laemmli loading buffer before being transferred onto western blot membranes (Immobilon-P PVDF membrane, EMD Millipore; Mini Trans-Blot cell, Bio-Rad) for 1 h at 90 V. Membranes were blocked using 5% milk in PBS+0.05% Tween-20 (PBST) overnight at 4° C. under agitation and probed with anti-OVA (ab181688) for 4 h at room temperature. Membranes were washed in PBST thrice, and incubated with a horseradish peroxidase (HRP)-conjugated anti-rabbit secondary antibody for 1 h at room temperature. Membranes were washed at least 3 times for 5 min in PBST, revealed using the Clarity Western ECL substrate (Bio-Rad) and imaged using a gel imaging system (Universal Hood III, Bio-Rad).

### 6. Mice

Female C57BL/6J (No 000664) or female MuMt-mice (B6.129S2-Ighm^{tm1Cgn}/J, No 002288) were between 8-12 weeks old at the start of the experiments, with mice being aged-matched within an experiment. Act-mOVA mice (C57BL/6-Tg(CAG-OVAL)916Jen/J, No 005145) were bred in-house and female mice of 25-35 week old were used for experimentation.

### 7. In Vivo Antibodies

All antibodies used in vivo were the InVivoMAb grade antibodies purchased from Bio X Cell (Lebanon, NH, USA). Antibodies used as immune checkpoint therapies were anti-PD-1 (clone 29F.1A12), anti-PD-L1 (clone 10F.9G2) and anti-CTLA-4 (clone 9H10). Antibodies used for immune cell depletion were anti-CD8α (clone 2.43), anti-CD4 (clone GK1.5), anti-NK1.1 (clone PK136), Isotype IgG2a (clone C1.18.4), Isotype IgG2b (clone LTF-2).

### 8. Tumour Injections

Mice were anaesthetised by isoflurane inhalation and were injected intradermally with 1.5 million of the different OVA-expressing or WT B16 cell lines. The tumour was measured using a digital calliper every 2 days, and tumour volume was calculated as follows: volume=length*width*height*(π/6). Mice were euthanised if sick or when the tumour volume reached 1 cm³. When indicated, mice were treated with immunotherapy, i.e. anti-PD-1 (200 μg) or the combination anti-PDL-1⁺ anti-CTLA-4 (100 μg each), once by intraperitoneal injection when the tumour volume was between 20-50 mm³ (day 5-8 post-tumour injection). When needed, 500 μg of depletion antibodies (anti-CD8α, anti-CD4, anti-NK1.1 or isotype control) were injected intraperitoneally 24 h after the checkpoint inhibitor therapy and repeated 7 days later. In the re-challenge experiments, 250 k WT B16 cells were injected intradermally on the contralateral side on the mice 1 month after they cleared the primary tumour.

### 9. Flow Cytometry Analysis of Tumour

Ten days after tumour injection, tumour were harvested on euthanised mice. Tumours were weighed, and about 300 mg were processed. Tumours were cut into small pieces, digested for 45 min in collagenase IV (1 mg/mL), DNAse I (40 μg/mL) in DMEM+2% FBS+1.2 mM CaCl₂) at 37° C. under magnetic stirring. The samples were pipetted 100 times to dissociate tumour pieces, and single cell suspensions were obtained by using 70 m cell strainer. Cells were kept on ice. Undigested pieces were further mixed with collagenase D (3.3 mg/mL), DNAse I (40 μg/mL) in DMEM+2% FBS+1.2 mM CaCl₂) for 30 min at 37° C., and collected as above. EDTA (5 mM) was added to the single cell suspension. The equivalent of 20 mg of tumour was used for staining for flow cytometry analysis. Tumour samples were washed in PBS and stained for cell viability for 15 min using Fixable Viability Dye eFluor 455UV (eBioscience, San Diego, CA, USA). The cells were washed and Fc receptors were blocked using anti-CD16/32 (#101302, BioLegend) for 20 min. Cells were then stained for 20 min on ice using the following antibodies: anti-CD45 (30-F11), anti-CD8α (53-6.7), anti-PD-1 (29F.1A12), anti-NK1.1 (PK136), anti-Ly6G (1A8), anti-Ly6C (HK1.4), anti-CD11b (M1/70), anti-F4/80 (BM8), anti-I-A/I-E (M5/114.15.2), from BioLegend (San Diego, CA, USA); anti-CD3ε (145-2C11), anti-CD4 (GK1.5), anti-CD62L (MEL-14), anti-CTLA-4 (UC10-4F10-11), anti-CD25 (PC61), anti-CD80 (16-10A1), anti-B220 (RA3-6B2), anti-CD19 (1D3), anti-CD11c (HL3), from BD Biosciences; anti-CD44 (IM7), anti-CD103 (2E7), from eBioscience. Cells were washed before analysis. When needed, intracellular staining with anti-FoxP3 (MF23, BD Biosciences) was performed using the BD Cytofix/Cytoperm Plus kit (BD Biosciences) according to the manufacturer's instruction. All staining procedures were done on ice with samples protected from light, in PBS+2% FBS+1 mM EDTA when not stated otherwise. Cells were analysed using a LSRFortessa flow cytometer (BD Biosciences). Data were processed using FlowJo (FlowJo LLC). Gating strategies for the flow analysis and biomarkers used to define cell populations are detailed in Supplementary Data 1.

### 10. Ex Vivo Antigen-Specific T Cell Restimulation

Ten days after tumour injection, spleens were harvested on euthanised mice. Single cell suspensions of splenocytes were obtained using a 70 m cell strainer. Cells were washed in PBS before the red blood cells were lysed in ACK buffer (Lonza, Basel, Switzerland) for 4 min and blocked with complete media (IMDM+10% FBS+1% P/S). Cells were centrifuged, resuspended in complete media, and 0.5 million were plated in in 96 U-bottom plate. OVA₂₅₇₋₂₆₄ (SIINFEKL; GenScript) and OVA₃₂₃₋₃₃₉ (ISQAVHAAHAEINEAGR; GenScript) were added to the splenocytes at a final concentration of 1 μg/mL to restimulate CD8⁺ and CD4⁺ T cells, respectively. Unstimulated controls were tested using complete media without peptide, and positive controls were tested using ionomycin (1 μg/mL)+PMA (50 ng/mL). After 4 days in culture, the cell supernatant was collected and the amount of IFNγ secreted was quantified using mouse IFNγ quantikine ELISA kit (R&D systems, Minneapolis, MN, USA) according to the manufacturer's instructions. Data represent the concentration of IFNγ secreted in restimulated culture supernatants subtracted with the amount detected in unstimulated supernatants.

### 11. IgG Titration in Plasma

Ten days after tumour injection, mice were bled by intracardiac puncture upon euthanasia. The blood was collected in EDTA-containing tubes, spun down at 1000 xg for 5 min and the plasma was collected and stored at −80° C. until analysis. ELISA plates (Maxisorp, Nunc, Roskilde, Denmark) were coated with 10 μg/mL OVA (Sigma-Aldrich, St. Louis, MO, USA) in PBS overnight at 4° C., and blocked with casein (Sigma-Aldrich, St. Louis, MO, USA) for 2 h at room temperature. The plates were washed with PBST, and plasma diluted in casein was added to the wells, starting at a concentration of 1:100 and serially diluted by 10, for 2 h at room temperature. The plates were washed again, and the following HRP-conjugated antibodies were used for detection: anti-mouse IgGI (#1070-05), anti-mouse IgG2a (#1080-05), anti-mouse IgG2b (#1090-05) and anti-mouse IgG3 (#1100-05) from Southern Biotech (Birmingham, AL, USA). The plates was revealed with TMB substrate (EMD Millipore) and stopped with 2N H₂SO4. Absorbance at 450 nm was read using an Epoch ELISA reader (BioTek, Winooski, VT, USA), and corrected by the absorbance at 570 nm. Antibody titers were determined as the highest plasma dilution for which the corrected absorbance was twice the background level. The area under curve (AUC) was calculated as area under the titration curve of the log₁₀(corrected absorbance over background).

### 12. Human Data Analysis

Processed sequencing data of tumour mutations (list of tumour mutated genes and tumour mutational burden score) and corresponding patient clinical data were obtained from the studies by Samstein et al.⁷, Hellman et al.⁸, and Hugo et al.⁹ Subcellular locations associated with Homo Sapiens genes (taxon ID=9606) were uploaded from UniProtKB/Swiss-Prot database on Aug. 2, 2020 and are provided in Supplementary Data 2 (Gene subcellular locations inventory). Algorithms for data processing and analysis were coded in R (Rstudio, Boston, MA, USA).

For each distinct tumour mutated gene of a patient, the inventors searched the gene name that matches in the gene subcellular locations inventory file. Genes that were not found were categorised as “Unfound genes”. Genes that were found but for which the subcellular location was unfound were further checked on the online UniprotKB/Swiss-Prot database using the GetSubcellular_location( ) function from the R package ‘UniprotR’. If the gene subcellular location remained unfound, it was then categorised as “Unknown location”. When multiple locations were found for a specific gene name, they were concatenated to obtain a single subcellular location entry per gene name. The gene subcellular locations were then categorised as membrane, cytoplasmic, nuclear or secreted by checking the presence of the character sequence: membrane=“Membrane” or “Cell membrane”, cytoplasmic=“Cytoplasm”, nuclear=“Nucleus”, secreted=“Secreted” in the subcellular location entry associated with the gene. When indicated, the categories were extended to the cell membrane=“Cell membrane”, the endoplasmic reticulum=“Reticulum” or “reticulum”, the Golgi apparatus=“Golgi” or “golgi”, or endosomal location=“Endosome” or “endosome” or “Endosomal” or “endosomal”. Oftentimes, a single gene name was associated with several subcellular locations, in which case the gene was included in several category in an non-exclusive way. For each patient, the inventors counted the number of mutated genes at each specific subcellular locations, and the proportion of mutated genes at a specific location was computed as the “number of tumour mutated genes at a location divided by the total number of tumour mutated genes in the patient”. Patients with no tumour mutated genes were removed from the analysis. In the presented data, groups of patients were determined using inclusive percentiles, except in groups separated at the median, for which the group below median was inclusive and the group above median was exclusive. In FIG. 5, “mAg High” and “mAg Low” correspond to groups of patients for which the proportion of mAg was respectively higher and lower than the median.

In R, survival analysis were performed using the libraries ‘survival’, ‘survminer’ and ‘survcomp’ and the functions survfit( ) and surv_pvalue( ). Hazard ratios were computed using the function hazard.ratio( ). All these functions were used using log-rank tests when asked. In case of multiple comparisons, p-values were adjusted using the function p.adjust( ).

### 13. Analysis of the Membrane-Localised Biomarkers

HR of survival was computed for each membrane protein-encoding gene, between patients that bear mutated version of the gene vs. patients that bear the wild-type version of the gene. The analysis was done independently for each cancer type. Results were considered relevant to report (in FIG. 5) when at least 7 patients with a mutated version of a gene were available, and 1) when the HR was ≤0.5 or statistically significance by the log-rank test was reached, or 2) when the HR ratio was ≥1.3 and close to statistically significance (p-value<0.2). The non ICI-treated cohort results were reported when at least 7 patients had the mutated version of the gene of interest.

### 14. Statistics & Softwares

Graphs were plotted using Prism 9 (GraphPad, San Diego, CA, USA). Statistical analysis were run on Prism 9 or on R (RStudio). Overall threshold for statistical significance was considered as p-value<0.05. Figures were made on Illustrator CS5 (Adobe, San Jose, CA, USA). Material, data and code availability

Tumour mutation sequencing data for the human cohorts used in this study are publicly available from Samstein et al. (7), Hellman et al. (8) and Hugo et al. (9). Subcellular locations associated to Homo Sapiens genes are provided in Supplementary Data 1 and updated versions can be downloaded from the UniProtKB/Swiss-Prot database.

D. Tables

Supplemental TABLE 1
ICI-treated cohort

| | | | | | | | Cumulated | Cumulated |
|---|---|---|---|---|---|---|---|---|
| | | | | | | | number | frequency of the |
| | | | | | | | of patients | mutated genes |
| | | | | | | Frequency of | with | in % |
| Mutated | Hazard | 95% CI | 95% CI | | Number | mutated | mutated | (patients |
| gene name | ratio | upper limit | lower limit | p-value | of patients | gene (%) | genes* | coverage) |
| BLADDER CANCER | | | | | 211 | | | |
| ALK | 0.2102 | 1.5091 | 0.0293 | 0.0868 | 9 | 4.27 | 9.00 | 4.27 |
| KDR | 0.2755 | 1.1197 | 0.0678 | 0.0537 | 12 | 5.69 | 19.00 | 9.00 |
| PTPRD | 0.2943 | 1.1957 | 0.0724 | 0.0690 | 11 | 5.21 | 26.00 | 12.32 |
| FAT1 | 0.4632 | 1.0625 | 0.2020 | 0.0627 | 21 | 9.95 | 43.00 | 20.38 |
| ERBB3 | 0.4862 | 1.1141 | 0.2122 | 0.0816 | 25 | 11.85 | 60.00 | 28.44 |
| NOTCH1** | 1.5511 | 2.9935 | 0.8037 | 0.1872 | 18 | 8.53 | — | — |
| BREAST CANCER | 0.3591 | 1.5746 | 0.0819 | 0.1590 | 41 | 9.76 | | |
| ESR1 | | | | | 4 | | | |
| COLORECTAL CANCER | | | | | 109 | | | |
| RNF43 | 0.1183 | 0.4941 | 0.0283 | 0.0005 | 21 | 19.27 | 21.00 | 19.27 |
| NOTCH3 | 0.1747 | 0.7374 | 0.0414 | 0.0080 | 16 | 14.68 | 26.00 | 23.85 |
| INPP4A | 0.1848 | 1.3516 | 0.0253 | 0.0623 | 7 | 6.42 | 27.00 | 24.77 |
| PTPRD | 0.2610 | 1.8997 | 0.0359 | 0.1532 | 8 | 7.34 | 31.00 | 28.44 |
| PDGFRB | 0.2668 | 1.1524 | 0.0618 | 0.0606 | 9 | 8.26 | 32.00 | 29.36 |
| PTCH1 | 0.2721 | 0.8974 | 0.0825 | 0.0229 | 17 | 15.60 | 36.00 | 33.03 |
| NOTCH4 | 0.3270 | 1.3615 | 0.0786 | 0.1063 | 10 | 9.17 | 37.00 | 33.94 |
| FAT1 | 0.3590 | 1.1624 | 0.1109 | 0.0744 | 18 | 16.51 | 40.00 | 36.70 |
| BRAF | 0.4121 | 1.3418 | 0.1266 | 0.1289 | 13 | 11.93 | 45.00 | 41.28 |
| CARD11** | 2.2448 | 5.4180 | 0.9301 | 0.0653 | 9 | 8.26 | — | — |
| APC** | 2.5055 | 5.6271 | 1.1156 | 0.0213 | 80 | 73.39 | — | — |
| CSF3R** | 3.2417 | 8.3119 | 1.2643 | 0.0096 | 7 | 6.42 | — | — |
| ESOPHAGOGASTRIC CANCER | | | | | 118 | | | |
| ATR | 0.2040 | 1.5218 | 0.0273 | 0.0887 | 5 | 4.24 | | |
| KRAS | 0.2775 | 1.1668 | 0.0660 | 0.0629 | 7 | 5.93 | | |
| NOTCH1** | 2.1042 | 5.8789 | 0.7531 | 0.1465 | 6 | 5.08 | | |
| GLIOMA | | | | | 116 | | | |
| FGFR1 | 0.2802 | 2.0145 | 0.0390 | 0.1767 | 4 | 3.45 | | |
| TSC2** | 2.0841 | 5.7861 | 0.7507 | 0.1495 | 5 | 4.31 | | |
| HEAD AND NECK CANCER | | | | | 129 | | | |
| EPHA7 | 0.3456 | 2.4921 | 0.0479 | 0.2694 | 5 | 3.88 | | |
| MAP2K2 | 0.3519 | 2.5392 | 0.0488 | 0.2786 | 4 | 3.10 | | |
| EPHB1 | 0.3632 | 2.6193 | 0.0503 | 0.2945 | 4 | 3.10 | | |
| ROS1** | 3.2215 | 8.2399 | 1.2595 | 0.0098 | 7 | 5.43 | | |
| NON-SMALL CELL LUNG CANCER | | | | | 344 | | | |
| FGFR4 | 0.2403 | 0.9674 | 0.0597 | 0.0292 | 9 | 2.62 | 9.00 | 2.62 |
| FLT3 | 0.2546 | 1.0250 | 0.0632 | 0.0376 | 9 | 2.62 | 17.00 | 4.94 |
| RET | 0.3352 | 1.0483 | 0.1072 | 0.0484 | 10 | 2.91 | 26.00 | 7.56 |
| EPHA7 | 0.3410 | 0.8283 | 0.1404 | 0.0127 | 18 | 5.23 | 40.00 | 11.63 |
| NTRK3 | 0.3616 | 0.8145 | 0.1605 | 0.0104 | 20 | 5.81 | 51.00 | 14.83 |
| MET | 0.3725 | 0.9050 | 0.1533 | 0.0232 | 14 | 4.07 | 61.00 | 17.73 |
| NOTCH1 | 0.3930 | 0.9546 | 0.1618 | 0.0325 | 14 | 4.07 | 70.00 | 20.35 |
| NOTCH2 | 0.4414 | 1.1877 | 0.1640 | 0.0960 | 11 | 3.20 | 76.00 | 22.09 |
| IL7R | 0.4527 | 1.2181 | 0.1682 | 0.1073 | 10 | 2.91 | 80.00 | 23.26 |
| EPHA5 | 0.4785 | 0.8786 | 0.2606 | 0.0150 | 29 | 8.43 | 98.00 | 28.49 |
| ERBB4 | 0.5568 | 1.0860 | 0.2855 | 0.0814 | 21 | 6.10 | 107.00 | 31.10 |
| EPHA3 | 0.5868 | 0.9633 | 0.3574 | 0.0330 | 37 | 10.76 | 130.00 | 37.79 |
| PTPRD | 0.6308 | 0.9902 | 0.4019 | 0.0433 | 43 | 12.50 | 144.00 | 41.86 |
| STK11** | 1.2969 | 1.7886 | 0.9404 | 0.1119 | 70 | 20.35 | — | — |
| TGFBR2** | 2.2149 | 4.7116 | 1.0412 | 0.0340 | 8 | 2.33 | — | — |
| MELANOMA | | | | | 313 | | | |
| IGF1R | 0.3410 | 0.9258 | 0.1256 | 0.0268 | 22 | 7.03 | 22.00 | 7.03 |
| ATR | 0.3911 | 1.2306 | 0.1243 | 0.0960 | 21 | 6.71 | 39.00 | 12.46 |
| INSR | 0.4038 | 1.2705 | 0.1283 | 0.1087 | 18 | 5.75 | 54.00 | 17.25 |
| NTRK2 | 0.4178 | 1.3144 | 0.1328 | 0.1235 | 16 | 5.11 | 62.00 | 19.81 |
| CARD11 | 0.4579 | 0.9388 | 0.2234 | 0.0288 | 38 | 12.14 | 82.00 | 26.20 |
| ERG | 0.4646 | 1.2604 | 0.1713 | 0.1230 | 20 | 6.39 | 92.00 | 29.39 |
| NTRK3 | 0.5070 | 1.0881 | 0.2362 | 0.0756 | 30 | 9.58 | 101.00 | 32.27 |
| PTPRD | 0.5582 | 0.9450 | 0.3298 | 0.0277 | 62 | 19.81 | 121.00 | 38.66 |
| ROS1 | 0.5816 | 0.9845 | 0.3436 | 0.0411 | 63 | 20.13 | 137.00 | 43.77 |
| PTPRT | 0.6190 | 0.9557 | 0.4010 | 0.0289 | 85 | 27.16 | 157.00 | 50.16 |
| BAP1** | 2.1472 | 4.2594 | 1.0824 | 0.0251 | 18 | 5.75 | — | — |
| GNAQ** | 2.1731 | 4.0457 | 1.1673 | 0.0121 | 17 | 5.43 | — | — |
| MAP2K2** | 2.3975 | 5.4623 | 1.0523 | 0.0317 | 8 | 2.56 | — | — |
| GNA11** | 2.4986 | 5.3815 | 1.1601 | 0.0154 | 10 | 3.19 | — | — |
| RENAL CELL CARCINOMA | | | | | 143 | | | |
| VHL | 0.3634 | 0.6411 | 0.2060 | 0.0003 | 106 | 74.13 | | |

Non-ICI-treated cohort

| Mutated | Hazard | 95% CI | 95% CI | | Number | Frequency of |
|---|---|---|---|---|---|---|
| gene name | ratio | upper limit | lower limit | p-value | of patients | mutated gene (%) |
| BLADDER CANCER | | | | | 50 | |
| ERBB3 | 0.5628 | 1.6142 | 0.1962 | 0.2785 | 7 | 14.00 |
| BREAST CANCER | | | | | 471 | |
| ESR1 | 0.8208 | 1.2749 | 0.5285 | 0.3787 | 68 | 14.44 |
| COLORECTAL CANCER | | | | | 362 | |
| RNF43 | 2.2292 | 4.0583 | 1.2245 | 0.0071 | 22 | 6.08 |
| NOTCH3 | 1.2079 | 2.1255 | 0.6864 | 0.5118 | 28 | 7.73 |
| INPP4A | 0.3475 | 2.4941 | 0.0484 | 0.2710 | 7 | 1.93 |
| PTPRD | 2.3734 | 5.4093 | 1.0414 | 0.0339 | 11 | 3.04 |
| PTCH1 | 1.8342 | 4.5298 | 0.7427 | 0.1819 | 9 | 2.49 |
| NOTCH4 | 0.4998 | 2.0272 | 0.1232 | 0.3220 | 12 | 3.31 |
| FAT1 | 0.6228 | 1.5462 | 0.2508 | 0.3031 | 18 | 4.97 |
| BRAF | 2.8178 | 4.3261 | 1.8354 | 7.83E−07 | 47 | 12.98 |
| CARD11** | 0.2888 | 2.0688 | 0.0403 | 0.1877 | 11 | 3.04 |
| APC** | 0.5689 | 0.8406 | 0.3850 | 0.0041 | 283 | 78.18 |
| ESOPHAGOGASTRIC CANCER | | | | | 132 | |
| ATR | 1.5759 | 6.4635 | 0.3843 | 0.5240 | 3 | 2.27 |
| KRAS | 0.1524 | 1.1347 | 0.0205 | 0.0371 | 6 | 4.55 |
| NOTCH1** | 0.5461 | 1.3902 | 0.2145 | 0.1986 | 11 | 8.33 |
| GLIOMA | | | | | 109 | |
| HEAD AND NECK CANCER | | | | | 33 | |
| NON-SMALL CELL LUNG CANCER | | | | | 411 | |
| FGFR4 | 0.7166 | 2.2577 | 0.2275 | 0.5675 | 7 | 1.70 |
| FLT3 | 0.9525 | 2.5679 | 0.3533 | 0.9233 | 10 | 2.43 |
| RET | 1.2111 | 2.7346 | 0.5364 | 0.6444 | 13 | 3.16 |
| EPHA7 | 1.0415 | 2.3584 | 0.4599 | 0.9223 | 13 | 3.16 |
| NTRK3 | 1.9666 | 3.6247 | 1.0670 | 0.0271 | 17 | 4.14 |
| MET | 0.7484 | 1.5944 | 0.3512 | 0.4510 | 19 | 4.62 |
| NOTCH1 | 3.4011 | 6.6954 | 1.7277 | 0.0002 | 10 | 2.43 |
| NOTCH2 | 2.2897 | 5.1705 | 1.0139 | 0.0403 | 8 | 1.95 |
| IL7R | 1.0371 | 3.2490 | 0.3311 | 0.9501 | 7 | 1.70 |
| EPHA5 | 1.1882 | 2.0506 | 0.6885 | 0.5351 | 27 | 6.57 |
| ERBB4 | 1.5836 | 2.7908 | 0.8985 | 0.1087 | 25 | 6.08 |
| EPHA3 | 1.0896 | 2.0051 | 0.5921 | 0.7827 | 23 | 5.60 |
| PTPRD | 1.1904 | 1.8767 | 0.7550 | 0.4525 | 40 | 9.73 |
| STK11** | 2.4700 | 3.6029 | 1.6933 | 1.21E−06 | 46 | 11.19 |
| TGFBR2** | 0.4147 | 1.6713 | 0.1029 | 0.2012 | 9 | 2.19 |
| MELANOMA | | | | | 17 | |
| RENAL CELL CARCINOMA | | | | | 41 | |
| VHL | 0.3461 | 1.0071 | 0.1190 | 0.0420 | 19 | 46.34 |

Supplementary Table 1. Hazard ratio of survival per mutated genes and per cancer type for the ICI and non-ICI treated patients cohort Samstein et al.*i.e., line 1->number of patients with ALK mutations only; line 2->number of patients with ALK or KDR mutations; line 3->number of patients with ALK or KDR or PTPRD mutations, etc . . . . . ** Gene markers for which the hazard ratio of survival is >1 when the gene is mutated in the ICI-treated cohort (indicating that mutation of these genes is a bad prognosis to ICI).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps ofthe method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the publications referred to throughout the specification, to the extent that they provide exemplary procedural or other details supplementary to those set forth herein, are specifically incorporated herein by reference.
1. Peters, S., Reck, M., Smit, E. F., Mok, T. & Hellmann, M. D. How to make the best use of immunotherapy as first-line treatment of advanced/metastatic non-small-cell lung cancer. Annals of Oncology 30, 884-896 (2019).
2. Murciano-Goroff, Y. R., Warner, A. B. & Wolchok, J. D. The future of cancer immunotherapy: microenvironment-targeting combinations. Cell Research 1-13 (2020). doi:10.1038/s41422-020-0337-2
3. Vaddepally, R. K., Kharel, P., Pandey, R., Garje, R. & Chandra, A. B. Review of Indications of FDA-Approved Immune Checkpoint Inhibitors per NCCN Guidelines with the Level of Evidence. Cancers 12, 738 (2020).
4. Robert, C. A decade of immune-checkpoint inhibitors in cancer therapy. Nature Communications 11, 3801-3 (2020).
5. Robert, C. et al. Durable Complete Response After Discontinuation of Pembrolizumab in Patients With Metastatic Melanoma. J Clin Oncol 36, 1668-1674 (2018).
6. Depil, S. Cold Tumors: A Therapeutic Challenge for Immunotherapy. 1-10 (2019). doi:10.3389/fimmu.2019.00168
7. Samstein, R. M. et al. Tumor mutational load predicts survival after immunotherapy across multiple cancer types. Nat Genet 51, 202-206 (2019).
8. Hellmann, M. D. et al. Genomic Features of Response to Combination Immunotherapy in Patients with Advanced Non-Small-Cell Lung Cancer. Cancer Cell 33, 843-852.e4 (2018).
9. Hugo, W. et al. Genomic and Transcriptomic Features of Response to Anti-PD-1 Therapy in Metastatic Melanoma. Cell 165, 35-44 (2016).
10. Keenan, T. E., Burke, K. P. & Van Allen, E. M. Genomic correlates of response to immune checkpoint blockade. Nat. Med. 1-14 (2019). doi:10.1038/s41591-019-0382-x
11. Tran, E., Robbins, P. F. & Rosenberg, S. A. ‘Final common pathway’ of human cancer immunotherapy: targeting random somatic mutations. Nat. Immunol. 18, 255-262 (2017).
12. Galluzzi, L., Chan, T. A., Kroemer, G., Wolchok, J. D. & López-Soto, A. The hallmarks of successful anticancer immunotherapy. Sci Transl Med 10, (2018).
13. Wells, D. K. et al. Key Parameters of Tumor Epitope Immunogenicity Revealed Through a Consortium Approach Improve Neoantigen Prediction. Cell 183, 818-834.e13 (2020).
14. Yarchoan, M., Johnson, B. A., Lutz, E. R., Laheru, D. A. & Jaffee, E. M. Targeting neoantigens to augment antitumour immunity. Nat Rev Cancer 17, 209-222 (2017).
15. Blum, J. S., Wearsch, P. A. & Cresswell, P. Pathways of antigen processing. Annu. Rev. Immunol. 31, 443-473 (2013).
16. Neefjes, J., Jongsma, M. L. M., Paul, P. & Bakke, O. Towards a systems understanding of MHC class I and MHC class II antigen presentation. Nat Rev Immunol 1-14 (2011). doi:10.1038/nri3084
17. Vyas, J. M., Van der Veen, A. G. & Ploegh, H. L. The known unknowns of antigen processing and presentation. Nat Rev Immunol 8, 607-618 (2008).
18. Briquez, P. S. et al. Engineering Targeting Materials for Therapeutic Cancer Vaccines. Front Bioeng Biotechnol 8, 19 (2020).
19. Almagro, J. C., Daniels-Wells, T. R., Perez-Tapia, S. M. & Penichet, M. L. Progress and Challenges in the Design and Clinical Development of Antibodies for Cancer Therapy. Front. Immunol. 8, 495-19 (2018).
20. UniProt Consortium. UniProt: the universal protein knowledgebase in 2021. Nucleic Acids Res. 49, D480-D489 (2021).
21. DiLillo, D. J., Yanaba, K. & Tedder, T. F. B cells are required for optimal CD4+ and CD8+ T cell tumor immunity: therapeutic B cell depletion enhances B16 melanoma growth in mice. The Journal of Immunology 184, 4006-4016 (2010).
22. Zehir, A. et al. Mutational landscape of metastatic cancer revealed from prospective clinical sequencing of 10,000 patients. Nat. Med. 23, 703-713 (2017).
23. Zhang, N., Shi, X., Ju, W., Lou, Y. & Luo, X. Rnf43 Mutation As A Biomarker For Immune Checkpoint Inhibitor Efficacy In Colorectal Cancer. (2021). doi:10.21203/rs.3.rs-536739/vl
24. Chakravarty, D. et al. OncoKB: A Precision Oncology Knowledge Base. JCO precision oncology 2017, (2017).
25. Kopetz, S. et al. Encorafenib, Binimetinib, and Cetuximab in BRAF V600E-Mutated Colorectal Cancer. N Engl J Med 381, 1632-1643 (2019).
26. Paik, P. K. et al. Tepotinib in Non-Small-Cell Lung Cancer with MET Exon 14 Skipping Mutations. N Engl J Med 383, 931-943 (2020).
27. Gautschi, O. et al. Targeting RET in Patients With RET-Rearranged Lung Cancers: Results From the Global, Multicenter RET Registry. J Clin Oncol 35, 1403-1410 (2017).
28. U.S. Food and Drug Administration. FDA approves pembrolizumab for adults and children with TMB-H solid tumors. 1-3 (2021). Available at: (Accessed: 5 Jul. 2021)
29. Allegretti, M. et al. Tearing down the walls: FDA approves next generation sequencing (NGS) assays for actionable cancer genomic aberrations. J. Exp. Clin. Cancer Res. 37, 47-3 (2018).
30. Rusch, M. et al. Clinical cancer genomic profiling by three-platform sequencing of whole genome, whole exome and transcriptome. Nature Communications 9, 3962-13 (2018).
31. U.S. Food and Drug Administration. U.S. FDA 510(k) clearance K190661: Omics Core NantHealth. 1-182 (2019). (Accessed: 22 Apr. 2021)
32. U.S. Food and Drug Administration. U.S. FDA 510(k) clearance K192073: Helix OpCo, LLC. 1-19 (2021). (Accessed: 22 Apr. 2021)
33. Alspach, E. et al. MHC-II neoantigens shape tumour immunity and response to immunotherapy. Nature 574, 696-701 (2019).
34. Corthay, A., Lundin, K. U., Lorvik, K. B., Hofgaard, P. O. & Bogen, B. Secretion of Tumor-Specific Antigen by Myeloma Cells Is Required for Cancer Immunosurveillance by CD4+ T Cells. Cancer Res. 69, 5901-5907 (2009).
35. Henning, P. et al. The subcellular location of antigen expressed by adenoviral vectors modifies adaptive immunity but not dependency on cross-presenting dendritic cells. Eur. J. Immunol. 41, 2185-2196 (2011).
36. Sedlik, C. et al. Different immunogenicity but similar antitumor efficacy of two DNA vaccines coding for an antigen secreted in different membrane vesicle-associated forms. J Extracell Vesicles 3, (2014).
37. Mitchell, J. P., Court, J., Mason, M. D., Tabi, Z. & Clayton, A. Increased exosome production from tumour cell cultures using the Integra CELLine Culture System. J. Immunol. Methods 335, 98-105 (2008).

1. A method for treating cancer in a subject comprising administering to the subject an immune checkpoint inhibitor to a subject that has been determined to have high membrane-localized antigens (mAg) relative to a control, wherein the control is a level of mAg in a biological sample from a subject or the average level of mAg in biological samples from subjects determined to not have an effective response to immunotherapy.

2. A method for treating cancer in a subject comprising administering to the subject an immunotherapy after a biological sample from the subject has been analyzed for membrane-localized antigens (mAg).

3. The method of claim 2, wherein the immunotherapy comprises immune checkpoint immunotherapy (ICI).

4. The method of claim 2 or 3, wherein the biological sample has been determined to have high mAg.

5. The method of claim 4, wherein the biological sample has been determined to have high mAg relative to a control, wherein the control is a cut-off value or wherein the control is level of mAg in a biological sample from a subject or the average level of mAg in biological samples from subjects determined to not have an effective response to immunotherapy.

6. The method of any one of claims 2-5, wherein the cancer is thyroid cancer, breast cancer, bladder cancer, cervical cancer, colon cancer, head and neck cancer, Hodgkin lymphoma, liver cancer, lung cancer, renal cancer, renal cell cancer, skin cancer, stomach cancer, esophagogastric cancer, glioma, non-small cell lung cancer, melanoma, or rectal cancer.

7. The method of any one of claims 2-6, wherein the cancer comprises a solid tumor.

8. The method of any one of claims 2-7, wherein the method further comprises administering at least one additional anticancer treatment.

9. The method of claim 8, wherein the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

10. The method of any one of claims 3-9, wherein the ICI therapy comprises a monotherapy or a combination ICI therapy.

11. The method of claim 10, wherein the ICI therapy comprises an inhibitor of PD-1, PDL1, PDL2, CTLA-4, B7-1, B7-2, and combinations thereof.

12. The method of any one of claims 10-11, wherein the ICI therapy comprises an anti-PD-1 monoclonal antibody, an anti-CTLA-4 monoclonal antibody, and combinations thereof.

13. The method of claim 12, wherein the ICI therapy comprises one or more of nivolumab, pembrolizumab, pidilizumab, ipilimumab or tremelimumab.

14. The method of any one of claims 2-13, wherein analyzing mAg comprises sequencing of nucleic acids in or performing immunohistochemistry on a biological sample from the subject.

15. The method of any one of claims 2-14, wherein the total tumor mutational burden (TMB) has been analyzed in a biological sample from the subject.

16. The method of claim 15, wherein the biological sample has been determined to have high TMB.

17. The method of claim 16, wherein the biological sample has been determined to have high TMB relative to a control.

18. The method of claim 17, wherein the control comprises the level of TMB in a biological sample from a subject or the average level of TMB in biological samples from subjects determined to not have an effective response to immunotherapy.

19. The method of claim 17, wherein the control comprises the TMB in non-cancerous tissue.

20. The method of any one of claims 15-19, wherein TMB has been determined by sequencing of nucleic acids in or performing immunohistochemistry on a biological sample from the subject.

21. The method of any one of claims 2-20, wherein the biological sample comprises a tissue sample, a cancerous sample, a tumor sample, or a sample obtained from a biopsy.

22. A method for treating cancer in a subject comprising administering to the subject an immunotherapy after the mutation status of a gene selected from the genes of Table 1 has been determined in a biological sample from the subject.

23. The method of claim 22, wherein the immunotherapy comprises immune checkpoint blockade (ICI) therapy.

24. The method of claim 22 or 23, wherein the biological sample has been determined to have (i) mutant ALK, KDR, PTPRD, FAT1, and/or ERBB3; (ii) non-mutant NOTCH1; or iii) combinations of (i) and/or (ii).

25. The method of claim 24, wherein the subject has bladder cancer.

26. The method of claim 22, wherein the biological sample has been determined to have mutant ESR1.

27. The method of claim 26, wherein the subject has breast cancer.

28. The method of claim 22, wherein the biological sample has been determined to have (i) mutant RNF43, NOTCH3, INPP4A, PTPRD, PDGFRB, PTCH1, NOTCH4, FAT1, and/or BRAF; (ii) non-mutant CARD11, APC, and/or CSF3R; or iii) combinations of (i) and/or (ii).

29. The method of claim 28, wherein the subject has colorectal cancer.

30. The method of claim 22, wherein the biological sample has been determined to have (i) mutant ATR and/or KRAS; (ii) non-mutant NOTCH1; or iii) combinations of (i) and/or (ii).

31. The method of claim 30, wherein the subject has esophagogastric cancer.

32. The method of claim 22, wherein the biological sample has been determined to have (i) mutant FGFR1; (ii) non-mutant TSC2; or iii) combination of (i) and (ii).

33. The method of claim 32, wherein the subject has glioma.

34. The method of claim 22, wherein the biological sample has been determined to have (i) mutant EPHA7, MAP2K2, and/or EPHB1; (ii) non-mutant ROS1; or iii) combinations of (i) and/or (ii).

35. The method of claim 34, wherein the subject has head and neck cancer.

36. The method of claim 22, wherein the biological sample has been determined to have (i) mutantFGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2; or iii) combinations of (i) and/or (ii).

37. The method of claim 36, wherein the subject has non-small cell lung cancer.

38. The method of claim 22, wherein the biological sample has been determined to have (i) mutant IGF1R, ATR, INSR, NTRK2, CARD11, ERG, NTRK3, PTPRD, ROS1, and/or PTPRT; (ii) non-mutant BAPI, GNAQ, MAP2K2, and/or GNA11; or iii) combinations of (i) and/or (ii).

39. The method of claim 38, wherein the subject has melanoma.

40. The method of claim 22, wherein the biological sample has been determined to have mutant VHL

41. The method of claim 40, wherein the subject has renal cell carcinoma.

42. The method of any one of claims 22-41, wherein the cancer comprises a solid tumor.

43. The method of any one of claims 22-42, wherein the method further comprises administering at least one additional anticancer treatment.

44. The method of claim 43, wherein the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

45. The method of any one of claims 23-44, wherein the ICI therapy comprises a monotherapy or a combination ICI therapy.

46. The method of claim 45, wherein the ICI therapy comprises an inhibitor of PD-1, PDL1, PDL2, CTLA-4, B7-1, B7-2, and combinations thereof.

47. The method of any one of claims 45-46, wherein the ICI therapy comprises an anti-PD-1 monoclonal antibody, an anti-CTLA-4 monoclonal antibody, and combinations thereof.

48. The method of claim 47, wherein the ICI therapy comprises one or more of nivolumab, pembrolizumab, pidilizumab, ipilimumab or tremelimumab.

49. The method of any one of claims 22-48, wherein determining the mutation status comprises sequencing nucleic acids isolated from a biological sample from the subject.

50. The method of any one of claims 22-49, wherein the total tumor mutational burden (TMB) has been analyzed in a biological sample from the subject.

51. The method of claim 50, wherein the biological sample has been determined to have high TMB in the biological sample.

52. The method of claim 51, wherein the biological sample has been determined to have high TMB in the biological sample relative to a control.

53. The method of claim 52, wherein the control comprises the level of TMB in a biological sample from a subject or the average level of TMB in biological samples from subjects determined to not have an effective response to the immunotherapy.

54. The method of claim 52, wherein the control comprises the TMB in non-cancerous tissue.

55. The method of any one of claims 50-54, wherein TMB has been determined by sequencing of nucleic acids in or performing immunohistochemistry on a biological sample from the subject.

56. The method of any one of claims 22-55, wherein the method further comprises evaluating a biological sample from the subject for mAg.

57. The method of claim 56, wherein the biological sample has been determined to have high mAg.

58. The method of claim 57, wherein the biological sample has been determined to have high mAg relative to a control, wherein the control is a cut-off value or wherein the control is level of mAg in a biological sample from a subject or the average level of mAg in biological samples from subjects determined to not have an effective response to immunotherapy.

59. The method of any one of claims 22-58, wherein the biological sample comprises a tissue sample, a cancerous sample, a tumor sample, or a sample obtained from a biopsy.

60. A method for prognosing a subject having cancer or for predicting a cancer subject's response to immunotherapy, the method comprising evaluating a biological sample from the subject for mAg.

61. The method of claim 60, wherein the immunotherapy comprises ICI therapy.

62. The method of claim 60 or 61, wherein the biological sample has been evaluated as having high mAg.

63. The method of claim 62, wherein the subject is predicted to respond effectively to the immunotherapy when the biological sample from the subject has been evaluated as having high mAg.

64. The method of claim 63, wherein the subject is to respond effectively to the immunotherapy when the biological sample from the subject has been evaluated as having high mAg compared to a control.

65. The method of any one of claims 62-64, wherein the subject is predicted to have a favorable prognosis when the biological sample from the subject has been evaluated as having high mAg.

66. The method of claim 65, wherein the subject is predicted to have a favorable prognosis when the biological sample from the subject has been evaluated as having high mAg compared to a control.

67. The method of claim 60, wherein the biological sample has been evaluated as having low mAg.

68. The method of claim 67, wherein the subject is predicted to not respond effectively to the immunotherapy when the biological sample from the subject has been evaluated as having low mAg.

69. The method of claim 68, wherein the subject is to not respond effectively to the immunotherapy when the biological sample from the subject has been evaluated as having low mAg compared to a control.

70. The method of any one of claims 67-69, wherein the subject is predicted to have an unfavorable prognosis when the biological sample from the subject has been evaluated as having low mAg.

71. The method of claim 70, wherein the subject is predicted to have an unfavorable prognosis when the biological sample from the subject has been evaluated as having low mAg compared to a control.

72. The method of any one of claims 60-71, wherein the cancer is thyroid cancer, breast cancer, bladder cancer, cervical cancer, colon cancer, head and neck cancer, Hodgkin lymphoma, liver cancer, lung cancer, renal cancer, renal cell cancer, skin cancer, stomach cancer, esophagogastric cancer, glioma, non-small cell lung cancer, melanoma, or rectal cancer.

73. The method of any one of claims 60-72, wherein the cancer comprises a solid tumor.

74. The method of any one of claims 60-73, wherein the method further comprises administering at least one additional anticancer treatment.

75. The method of claim 74, wherein the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

76. The method of any one of claims 60-75, wherein the ICI therapy comprises a monotherapy or a combination ICI therapy.

77. The method of claim 76, wherein the ICI therapy comprises an inhibitor of PD-1, PDL1, PDL2, CTLA-4, B7-1, B7-2, and combinations thereof.

78. The method of any one of claims 76-77, wherein the ICI therapy comprises an anti-PD-1 monoclonal antibody, an anti-CTLA-4 monoclonal antibody, and combinations thereof.

79. The method of claim 78, wherein the ICI therapy comprises one or more of nivolumab, pembrolizumab, pidilizumab, ipilimumab or tremelimumab.

80. The method of any one of claims 60-79, wherein evaluating mAg comprises sequencing of nucleic acids in or performing immunohistochemistry on a biological sample from the subject.

81. The method of any one of claims 60-80, wherein the method further comprises evaluating the total tumor mutational burden (TMB) in a biological sample from the subject.

82. The method of claim 81, wherein the biological sample is evaluated as having a high TMB.

83. The method of claim 82, wherein the subject is predicted to respond effectively to the immunotherapy when the biological sample from the subject has been evaluated as having high mAg and high TMB.

84. The method of claim 82, wherein the subject is predicted to respond effectively to the immunotherapy when the biological sample from the subject has been evaluated as having high mAg and high TMB compared to a control.

85. The method of any one of claims 82-84, wherein the subject is predicted to have a favorable prognosis when the biological sample from the subject has been evaluated as having high mAg and high TMB.

86. The method of claim 85, wherein the subject is predicted to have a favorable prognosis when the biological sample from the subject has been evaluated as having high mAg and high TMB compared to a control.

87. The method of claim 81, wherein the biological sample has been evaluated as having low TMB.

88. The method of claim 87, wherein the subject is predicted to not respond effectively to the immunotherapy when the biological sample from the subject has been evaluated as having low mAg and low TMB.

89. The method of claim 88, wherein the subject is to not respond effectively to the immunotherapy when the biological sample from the subject has been evaluated as having low mAg and low TMB compared to a control.

90. The method of any one of claims 87-89, wherein the subject is predicted to have an unfavorable prognosis when the biological sample from the subject has been evaluated as having low mAg and low TMB.

91. The method of claim 90, wherein the subject is predicted to have an unfavorable prognosis when the biological sample from the subject has been evaluated as having low mAg and low TMB compared to a control.

92. The method of any one of claims 81-91, wherein TMB has been determined by by sequencing of nucleic acids in or performing immunohistochemistry on a biological sample from the subject.

93. The method of any one of claims 60-92, wherein the biological sample comprises a tissue sample, a cancerous sample, a tumor sample, or a sample obtained from a biopsy.

94. A method for predicting a cancer subject's response to immunotherapy, the method comprising analyzing the mutation status of a gene selected from the genes of Table 1 in a biological sample from the subject.

95. The method of claim 94, wherein the immunotherapy comprises ICI therapy.

96. The method of claim 94, wherein the method further comprises evaluating the total tumor mutational burden (TMB) in a biological sample from the subject.

97. The method of claim 94 or 96, wherein the subject has bladder cancer.

98. The method of claim 97, wherein the biological sample has been evaluated as having (i) mutant ALK, KDR, PTPRD, FAT1, and/or ERBB3; (ii) non-mutant NOTCH1; or iii) combinations of (i) and/or (ii).

99. The method of claim 97 or 98, wherein the subject is predicted to respond effectively to the immunotherapy when the biological sample from the subject has been evaluated as having (i) mutant ALK, KDR, PTPRD, FAT1, and/or ERBB3; (ii) non-mutant NOTCH1;
or iii) combinations of (i) and/or (ii).

100. The method of claim 97 or 98, wherein the subject is predicted to respond effectively to the immunotherapy when the biological sample from the subject has been evaluated as having high TMB and (i) mutant ALK, KDR, PTPRD, FAT1, and/or ERBB3; (ii) non-mutant NOTCH1; or iii) combinations of (i) and/or (ii).

101. The method of claim 97, wherein the biological sample has been evaluated as not having (i) mutant ALK, KDR, PTPRD, FAT1, and/or ERBB3; (ii) non-mutant NOTCH1; or iii) combinations of (i) and/or (ii).

102. The method of claim 97 or 101, wherein the subject is predicted to not respond effectively to the immunotherapy when the biological sample from the subject has been evaluated as not having (i) mutant ALK, KDR, PTPRD, FAT1, and/or ERBB3; (ii) non-mutant NOTCH1; or iii) combinations of (i) and/or (ii).

103. The method of claim 97 or 101, wherein the subject is predicted to not respond effectively to the immunotherapy when the biological sample from the subject has been evaluated as having low TMB and as not having (i) mutant ALK, KDR, PTPRD, FAT1, and/or ERBB3;
(ii) non-mutant NOTCH1; or iii) combinations of (i) and/or (ii).

104. The method of claim 94 or 96, wherein the subject has breast cancer.

105. The method of claim 104, wherein the biological sample has been evaluated as having mutant ESR1.

106. The method of claim 104 or 105, wherein the subject is predicted to respond effectively to the immunotherapy when the biological sample from the subject has been evaluated as having mutant ESR1.

107. The method of claim 104 or 105, wherein the subject is predicted to respond effectively to the immunotherapy when the biological sample from the subject has been evaluated as having high TMB and mutant ESR1.

108. The method of claim 104, wherein the biological sample has been evaluated as not having mutant ESR1.

109. The method of claim 104 or 108, wherein the subject is predicted to not respond effectively to the immunotherapy when the biological sample from the subject has been evaluated as not having mutant ESR1.

110. The method of claim 104 or 108, wherein the subject is predicted to not respond effectively to the immunotherapy when the biological sample from the subject has been evaluated as having low TMB and as not having mutant ESR1.

111. The method of claim 94 or 96, wherein the subject has colorectal cancer.

112. The method of claim 111, wherein the biological sample has been evaluated as having (i) mutant RNF43, NOTCH3, INPP4A, PTPRD, PDGFRB, PTCH1, NOTCH4, FAT1, and/or BRAF; (ii) non-mutant CARD11, APC, and/or CSF3R; or iii) combinations of (i) and/or (ii).

113. The method of claim 111 or 112, wherein the subject is predicted to respond effectively to the immunotherapy when the biological sample from the subject has been evaluated as having (i) mutant RNF43, NOTCH3, INPP4A, PTPRD, PDGFRB, PTCH1, NOTCH4, FAT1, and/or BRAF; (ii) non-mutant CARD11, APC, and/or CSF3R; or iii) combinations of (i) and/or (ii).

114. The method of claim 111 or 112, wherein the subject is predicted to respond effectively to the immunotherapy when the biological sample from the subject has been evaluated as having high TMB and as having (i) mutant RNF43, NOTCH3, INPP4A, PTPRD, PDGFRB, PTCH1, NOTCH4, FAT1, and/or BRAF; (ii) non-mutant CARD11, APC,
and/or CSF3R; or iii) combinations of (i) and/or (ii).

115. The method of claim 111, wherein the biological sample has been evaluated as not having (i) mutant RNF43, NOTCH3, INPP4A, PTPRD, PDGFRB, PTCH1, NOTCH4, FAT1,
and/or BRAF; (ii) non-mutant CARD11, APC, and/or CSF3R; or iii) combinations of (i) and/or (ii).

116. The method of claim 111 or 115, wherein the subject is predicted to not respond effectively to the immunotherapy when the biological sample from the subject has been evaluated as not having (i) mutant RNF43, NOTCH3, INPP4A, PTPRD, PDGFRB, PTCH1, NOTCH4, FAT1, and/or BRAF; (ii) non-mutant CARD11, APC, and/or CSF3R; or iii) combinations of (i) and/or (ii).

117. The method of claim 111 or 115, wherein the subject is predicted to not respond effectively to the immunotherapy when the biological sample from the subject has been evaluated as having low TMB and as not having (i) mutant RNF43, NOTCH3, INPP4A, PTPRD, PDGFRB, PTCH1, NOTCH4, FAT1, and/or BRAF; (ii) non-mutant CARD11, APC,
and/or CSF3R; or iii) combinations of (i) and/or (ii).

118. The method of claim 94 or 96, wherein the subject has esophagogastric cancer.

119. The method of claim 118, wherein the biological sample has been evaluated as having (i) mutant ATR and/or KRAS; (ii) non-mutant NOTCH1; or iii) combinations of (i) and/or (ii).

120. The method of claim 118 or 119, wherein the subject is predicted to respond effectively to the immunotherapy when the biological sample from the subject has been evaluated as having (i) mutant ATR and/or KRAS; (ii) non-mutant NOTCH1; or iii) combinations of (i) and/or (ii).

121. The method of claim 118 or 119, wherein the subject is predicted to respond effectively to the immunotherapy when the biological sample from the subject has been evaluated as having high TMB and as having (i) mutant ATR and/or KRAS; (ii) non-mutant NOTCH1;
or iii) combinations of (i) and/or (ii).

122. The method of claim 118, wherein the biological sample has been evaluated as not having (i) mutant ATR and/or KRAS; (ii) non-mutant NOTCH1; or iii) combinations of (i) and/or (ii).

123. The method of claim 118 or 122, wherein the subject is predicted to not respond effectively to the immunotherapy when the biological sample from the subject has been evaluated as not having (i) mutant ATR and/or KRAS; (ii) non-mutant NOTCH1; or iii) combinations of (i) and/or (ii).

124. The method of claim 118 or 122, wherein the subject is predicted to not respond effectively to the immunotherapy when the biological sample from the subject has been evaluated as having low TMB and as not having (i) mutant ATR and/or KRAS; (ii) non-mutant NOTCH1; or iii) combinations of (i) and/or (ii).

125. The method of claim 94 or 96, wherein the subject has glioma.

126. The method of claim 125, wherein the biological sample has been evaluated as having (i) mutant FGFR1; (ii) non-mutant TSC2; or iii) combination of (i) and (ii).

127. The method of claim 125 or 126, wherein the subject is predicted to respond effectively to the immunotherapy when the biological sample from the subject has been evaluated as having (i) mutant FGFR1; (ii) non-mutant TSC2; or iii) combination of (i) and (ii).

128. The method of claim 125 or 126, wherein the subject is predicted to respond effectively to the immunotherapy when the biological sample from the subject has been evaluated as having high TMB and as having (i) mutant FGFR1; (ii) non-mutant TSC2; or iii) combination of (i) and (ii).

129. The method of claim 125, wherein the biological sample has been evaluated as not having (i) mutant FGFR1; (ii) non-mutant TSC2; or iii) combination of (i) and (ii).

130. The method of claim 125 or 129, wherein the subject is predicted to not respond effectively to the immunotherapy when the biological sample from the subject has been evaluated as not having (i) mutant FGFR1; (ii) non-mutant TSC2; or iii) combination of (i) and (ii).

131. The method of claim 125 or 129, wherein the subject is predicted to not respond effectively to the immunotherapy when the biological sample from the subject has been evaluated as having low TMB and as not having (i) mutant FGFR1; (ii) non-mutant TSC2; or iii) combination of (i) and (ii).

132. The method of claim 94 or 96, wherein the subject has head and neck cancer.

133. The method of claim 132, wherein the biological sample has been evaluated as having (i) mutant EPHA7, MAP2K2, and/or EPHB1; (ii) non-mutant ROS1; or iii) combinations of (i) and/or (ii).

134. The method of claim 132 or 133, wherein the subject is predicted to respond effectively to the immunotherapy when the biological sample from the subject has been evaluated as having (i) mutant EPHA7, MAP2K2, and/or EPHB1; (ii) non-mutant ROS1; or iii) combinations of (i) and/or (ii).

135. The method of claim 132 or 133, wherein the subject is predicted to respond effectively to the immunotherapy when the biological sample from the subject has been evaluated as having high TMB and as having (i) mutant EPHA7, MAP2K2, and/or EPHB1; (ii) non-mutant ROS1; or iii) combinations of (i) and/or (ii).

136. The method of claim 132, wherein the biological sample has been evaluated as not having (i) mutant EPHA7, MAP2K2, and/or EPHB1; (ii) non-mutant ROS1; or iii) combinations of (i) and/or (ii).

137. The method of claim 132 or 136, wherein the subject is predicted to not respond effectively to the immunotherapy when the biological sample from the subject has been evaluated as not having (i) mutant EPHA7, MAP2K2, and/or EPHB1; (ii) non-mutant ROS1; or iii) combinations of (i) and/or (ii).

138. The method of claim 132 or 136, wherein the subject is predicted to not respond effectively to the immunotherapy when the biological sample from the subject has been evaluated as having low TMB and as not having (i) mutant EPHA7, MAP2K2, and/or EPHB1; (ii) non-mutant ROS1; or iii) combinations of (i) and/or (ii).

139. The method of claim 94 or 96, wherein the subject has non-small cell lung cancer.

140. The method of claim 139, wherein the biological sample has been evaluated as having (i) mutant FGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2; or iii) combinations of (i) and/or (ii).

141. The method of claim 139 or 140, wherein the subject is predicted to respond effectively to the immunotherapy when the biological sample from the subject has been evaluated as having (i) mutant FGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2;
or iii) combinations of (i) and/or (ii).

142. The method of claim 139 or 140, wherein the subject is predicted to respond effectively to the immunotherapy when the biological sample from the subject has been evaluated as having high TMB and as having (i) mutant FGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2; or iii) combinations of (i) and/or (ii).

143. The method of claim 139, wherein the biological sample has been evaluated as not having (i) mutant FGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2; or iii) combinations of (i) and/or (ii).

144. The method of claim 139 or 143, wherein the subject is predicted to not respond effectively to the immunotherapy when the biological sample from the subject has been evaluated as not having (i) mutant FGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2;
or iii) combinations of (i) and/or (ii).

145. The method of claim 139 or 143, wherein the subject is predicted to not respond effectively to the immunotherapy when the biological sample from the subject has been evaluated as having low TMB and as not having (i) mutant FGFR4, FLT3, RET, EPHA7, NTRK3, MET, NOTCH1, NOTCH2, IL7R, EPHA5, ERBB4, EPHA3, and/or PTPRD; (ii) non-mutant STK11 and/or TGFBR2; or iii) combinations of (i) and/or (ii).

146. The method of claim 94 or 96, wherein the subject has melanoma.

147. The method of claim 146, wherein the biological sample has been evaluated as having (i) mutant IGF1R, ATR, INSR, NTRK2, CARD11, ERG, NTRK3, PTPRD, ROS1, and/or PTPRT; (ii) non-mutant BAP1, GNAQ, MAP2K2, and/or GNA11; or iii) combinations of (i) and/or (ii).

148. The method of claim 146 or 147, wherein the subject is predicted to respond effectively to the immunotherapy when the biological sample from the subject has been evaluated as having (i) mutant IGF1R, ATR, INSR, NTRK2, CARD11, ERG, NTRK3, PTPRD, ROS1, and/or PTPRT; (ii) non-mutant BAP1, GNAQ, MAP2K2, and/or GNA11; or iii) combinations of (i) and/or (ii).

149. The method of claim 146 or 147, wherein the subject is predicted to respond effectively to the immunotherapy when the biological sample from the subject has been evaluated as having high TMB and as having (i) mutant IGF1R, ATR, INSR, NTRK2, CARD11, ERG, NTRK3, PTPRD, ROS1, and/or PTPRT; (ii) non-mutant BAP1, GNAQ, MAP2K2, and/or GNA11; or iii) combinations of (i) and/or (ii).

150. The method of claim 146, wherein the biological sample has been evaluated as not having (i) mutant IGF1R, ATR, INSR, NTRK2, CARD11, ERG, NTRK3, PTPRD, ROS1, and/or PTPRT; (ii) non-mutant BAPi, GNAQ, MAP2K2, and/or GNA11; or iii) combinations of (i) and/or (ii).

151. The method of claim 146 or 150, wherein the subject is predicted to not respond effectively to the immunotherapy when the biological sample from the subject has been evaluated as not having (i) mutant IGF1R, ATR, INSR, NTRK2, CARD11, ERG, NTRK3, PTPRD, ROS1, and/or PTPRT; (ii) non-mutant BAP1, GNAQ, MAP2K2, and/or GNA11; or iii) combinations of (i) and/or (ii).

152. The method of claim 146 or 150, wherein the subject is predicted to not respond effectively to the immunotherapy when the biological sample from the subject has been evaluated as having low TMB and as not having (i) mutant IGF1R, ATR, INSR, NTRK2, CARD11, ERG, NTRK3, PTPRD, ROS1, and/or PTPRT; (ii) non-mutant BAP1, GNAQ, MAP2K2, and/or GNA11; or iii) combinations of (i) and/or (ii).

153. The method of claim 94 or 96, wherein the subject has renal cell carcinoma.

154. The method of claim 153, wherein the biological sample has been evaluated as having mutant VHL

155. The method of claim 146 or 147, wherein the subject is predicted to respond effectively to the immunotherapy when the biological sample from the subject has been evaluated as having mutant VHL.

156. The method of claim 146 or 147, wherein the subject is predicted to respond effectively to the immunotherapy when the biological sample from the subject has been evaluated as having high TMB and as having mutant VHL.

157. The method of claim 153, wherein the biological sample has been evaluated as not having mutant VHL

158. The method of claim 146 or 157, wherein the subject is predicted to not respond effectively to the immunotherapy when the biological sample from the subject has been evaluated as not having mutant VHL.

159. The method of claim 146 or 157, wherein the subject is predicted to not respond effectively to the immunotherapy when the biological sample from the subject has been evaluated as having low TMB and as not having mutant VHL.

160. The method of any one of claims 94-159, wherein the cancer comprises a solid tumor.

161. The method of any one of claims 94-160, wherein the method further comprises administering at least one anticancer treatment.

162. The method of claim 161, wherein the at least one anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

163. The method of any one of claims 94-162, wherein the method further comprises administering an immunotherapy to the subject predicted to respond to the immunotherapy.

164. The method of any one of claims 94-162, wherein the method excludes administering an immunotherapy to the subject predicted to not respond to the immunotherapy.

165. The method of any one of claims 94-164, wherein the ICI therapy comprises a monotherapy or a combination ICI therapy.

166. The method of claim 165, wherein the ICI therapy comprises an inhibitor of PD-1, PDL1, PDL2, CTLA-4, B7-1, B7-2, and combinations thereof.

167. The method of any one of claims 165-166, wherein the ICI therapy comprises an anti-PD-1 monoclonal antibody, an anti-CTLA-4 monoclonal antibody, and combinations thereof.

168. The method of claim 167, wherein the ICI therapy comprises one or more of nivolumab, pembrolizumab, pidilizumab, ipilimumab or tremelimumab.

169. The method of any one of claims 94-168, wherein determining the mutation status comprises sequencing nucleic acids isolated from a biological sample from the subject.

170. The method of any one of claims 94-169, wherein the biological sample comprises a tissue sample, a cancerous sample, a tumor sample, or a sample obtained from a biopsy.

171. A method comprising evaluating mAg in a biological sample from a subject having cancer.

172. The method of claim 171, wherein the biological sample has been evaluated as having high mAg.

173. The method of claim 171, wherein the biological sample has been evaluated as having low mAg.

174. The method of claim 172 or 173, wherein the biological sample has been evaluated as having high or low mAg compared to a control.

175. The method of any one of claims 171-174, wherein the cancer is thyroid cancer, breast cancer, bladder cancer, cervical cancer, colon cancer, head and neck cancer, Hodgkin lymphoma, liver cancer, lung cancer, renal cancer, renal cell cancer, skin cancer, stomach cancer, esophagogastric cancer, glioma, non-small cell lung cancer, melanoma, or rectal cancer.

176. The method of any one of claims 171-175, wherein the cancer comprises a solid tumor.

177. The method of any one of claims 171-176, wherein the subject is being treated with at least one anticancer treatment.

178. The method of claim 177, wherein the at least one anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

179. The method of any one of claims 171-176, wherein the subject is not being treated for cancer.

180. The method of any one of claims 171-176, wherein the subject is being treated with an immunotherapy.

181. The method of any one of claims 171-176, wherein the subject is not being treated with an immunotherapy.

182. The method of claim 180 or 181, wherein the immunotherapy comprises ICI therapy.

183. The method of claim 182, wherein the ICI therapy comprises a monotherapy or a combination ICI therapy.

184. The method of claim 183, wherein the ICI therapy comprises an inhibitor of PD-1, PDL1, PDL2, CTLA-4, B7-1, B7-2, and combinations thereof.

185. The method of any one of claims 183-184, wherein the ICI therapy comprises an anti-PD-1 monoclonal antibody, an anti-CTLA-4 monoclonal antibody, and combinations thereof.

186. The method of claim 185, wherein the ICI therapy comprises one or more of nivolumab, pembrolizumab, pidilizumab, ipilimumab or tremelimumab.

187. The method of any one of claims 171-186, wherein evaluating mAg comprises sequencing of nucleic acids in or performing immunohistochemistry on a biological sample from the subject.

188. The method of any one of claims 171-187, wherein the method further comprises evaluating TMB in a biological sample from the subject.

189. The method of claim 188, wherein the biological sample is evaluated as having a high TMB.

190. The method of claim 188, wherein the biological sample is evaluated as having a low TMB.

191. The method of claim 189 or 190, wherein the subject is evaluated as having high or low TMB compared to a control.

192. The method of any one of claims 188-191, wherein TMB has been evaluated by sequencing of nucleic acids in or performing immunohistochemistry on a biological sample from the subject.

193. The method of any one of claims 171-192, wherein the biological sample comprises a tissue sample, a cancerous sample, a tumor sample, or a sample obtained from a biopsy.

194. A method comprising evaluating the mutation status of a gene selected from the genes of Table 1 in a biological sample from a subject having cancer.

195. The method of claim 194, wherein the method further comprises evaluating the total tumor mutational burden (TMB) in the biological sample from the subject.

196. The method of claim 194 or 195, wherein the subject has bladder cancer.

197. The method of claim 196, wherein the biological sample has been evaluated for the mutation status of ALK, KDR, PTPRD, FAT1, ERBB3, NOTCH1, and combinations thereof.

198. The method of claim 194 or 195, wherein the subject has breast cancer.

199. The method of claim 198, wherein the biological sample has been evaluated for the mutation status of ESR1.

200. The method of claim 194 or 195, wherein the subject has colorectal cancer.

201. The method of claim 200, wherein the biological sample has been evaluated for the mutation status of RNF43, NOTCH3, INPP4A, PTPRD, PDGFRB, PTCH1, NOTCH4, FAT1, BRAF, CARD11, APC, CSF3R, and combinations thereof.

202. The method of claim 194 or 195, wherein the subject has esophagogastric cancer.

203. The method of claim 202, wherein the biological sample has been evaluated for the mutation status of ATR, KRAS, NOTCH1, and combinations thereof.

204. The method of claim 194 or 195, wherein the subject has glioma.

205. The method of claim 204, wherein the biological sample has been evaluated for the mutation status of FGFR1, TSC2, and combinations thereof.

206. The method of claim 194 or 195, wherein the subject has head and neck cancer.

207. The method of claim 206, wherein the biological sample has been evaluated for the mutation status of EPHA7, MAP2K2, EPHB1, ROS1, and combinations thereof.

208. The method of claim 194 or 195, wherein the subject has non-small cell lung cancer.

209. The method of claim 208, wherein the biological sample has been evaluated for the mutation status of FGFR4, FLT3, RET, EPHA7, NTRK3, MET, NOTCH1, NOTCH2, IL7R, EPHA5, ERBB4, EPHA3, PTPRD, STK11, TGFBR2, and combinations thereof.

210. The method of claim 194 or 195, wherein the subject has melanoma.

211. The method of claim 210, wherein the biological sample has been evaluated for the mutation status of IGF1R, ATR, INSR, NTRK2, CARD11, ERG, NTRK3, PTPRD, ROS1, PTPRT, BAP1, GNAQ, MAP2K2, GNA11, and combinations thereof.

212. The method of claim 194 or 195, wherein the subject has renal cell carcinoma.

213. The method of claim 212, wherein the biological sample has been evaluated for the mutation status of VHL.

214. The method of any one of claims 194-213, wherein the cancer comprises a solid tumor.

215. The method of any one of claims 194-214, wherein the subject is being treated with at least one anticancer treatment.

216. The method of claim 215, wherein the at least one anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

217. The method of any one of claims 194-213, wherein the subject is not being treated for cancer.

218. The method of any one of claims 194-213, wherein the subject is being treated with an immunotherapy.

219. The method of any one of claims 194-213, wherein the subject is not being treated with an immunotherapy.

220. The method of claim 218 or 219, wherein the immunotherapy comprises ICI therapy.

221. The method of claim 220, wherein the ICI therapy comprises a monotherapy or a combination ICI therapy.

222. The method of claim 221, wherein the ICI therapy comprises an inhibitor of PD-1, PDL1, PDL2, CTLA-4, B7-1, B7-2, and combinations thereof.

223. The method of any one of claims 221-222, wherein the ICI therapy comprises an anti-PD-1 monoclonal antibody, an anti-CTLA-4 monoclonal antibody, and combinations thereof.

224. The method of claim 223, wherein the ICI therapy comprises one or more of nivolumab, pembrolizumab, pidilizumab, ipilimumab or tremelimumab.

225. The method of any one of claims 194-224, wherein evaluating the mutation status comprises sequencing nucleic acids isolated from a biological sample from the subject.

226. The method of any one of claims 194-225, wherein the method further comprises evaluating TMB in a biological sample from the subject.

227. The method of any one of claims 194-226, wherein TMB has been evaluated by sequencing of nucleic acids in or performing immunohistochemistry on a biological sample from the subject.

228. The method of any one of claims 194-227, wherein the biological sample comprises a tissue sample, a cancerous sample, a tumor sample, or a sample obtained from a biopsy.

229. A method for monitoring a response to an immunotherapy in a subject having cancer comprising: analyzing mAg in a biological sample from the subject before and/or after the subject has been treated with the ICI therapy.

230. The method of claim 229, wherein the biological sample has been evaluated as having high or low mAg compared to a control.

231. The method of any one of claims 229 or 230, wherein the cancer is thyroid cancer, breast cancer, bladder cancer, cervical cancer, colon cancer, head and neck cancer, Hodgkin lymphoma, liver cancer, lung cancer, renal cancer, renal cell cancer, skin cancer, stomach cancer, esophagogastric cancer, glioma, non-small cell lung cancer, melanoma, or rectal cancer.

232. The method of any one of claims 229-231, wherein the cancer comprises a solid tumor.

233. The method of any one of claims 229-232, wherein the subject is being treated with at least one anticancer treatment.

234. The method of claim 233, wherein the at least one anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

235. The method of any one of claims 229-232, wherein the subject is not being treated for cancer.

236. The method of any one of claims 229-232, wherein the subject is being treated with an immunotherapy.

237. The method of any one of claims 229-232, wherein the subject is not being treated with an immunotherapy.

238. The method of any one of claims 234-237, wherein the immunotherapy comprises ICI therapy.

239. The method of claim 238, wherein the ICI therapy comprises a monotherapy or a combination ICI therapy.

240. The method of claim 239, wherein the ICI therapy comprises an inhibitor of PD-1, PDL1, PDL2, CTLA-4, B7-1, B7-2, and combinations thereof.

241. The method of any one of claims 239-240, wherein the ICI therapy comprises an anti-PD-1 monoclonal antibody, an anti-CTLA-4 monoclonal antibody, and combinations thereof.

242. The method of claim 241, wherein the ICI therapy comprises one or more of nivolumab, pembrolizumab, pidilizumab, ipilimumab or tremelimumab.

243. The method of any one of claims 229-242, wherein evaluating mAg comprises sequencing of nucleic acids in or performing immunohistochemistry on a biological sample from the subject.

244. The method of any one of claims 229-243, wherein the method further comprises evaluating TMB in a biological sample from the subject.

245. The method of claim 244, wherein the biological sample is evaluated as having a high TMB.

246. The method of claim 244, wherein the biological sample is evaluated as having a low TMB.

247. The method of claim 245 or 246, wherein the subject is evaluated as having high or low TMB compared to a control.

248. The method of any one of claims 244-247, wherein TMB has been evaluated by sequencing of nucleic acids in or performing immunohistochemistry on a biological sample from the subject.

249. The method of any one of claims 229-248, wherein the biological sample comprises a tissue sample, a cancerous sample, a tumor sample, or a sample obtained from a biopsy.
